SEQUENCE LISTING (801 sequences)

SEQ ID NO: 1 - protein, 119 aa, synthetic construct
  REGION 1..119: note = RB4v1.0 VH
QVQLVQPGAEVVKPGASVKLSCKTSGYTFTSNWMHWVKQRPGQGLEWIGEIDPSDSYTNYNQNFKGKAKLTVDKSTSTAYMEVSSLRSDDTAVYYCARGSNPYYYAMDYWGQGTSVTVS

SEQ ID NO: 2 - protein, 119 aa, synthetic construct
  REGION 1..119: note = RB4v1.2 VH
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVS

SEQ ID NO: 3 - protein, 121 aa, synthetic construct
  REGION 1..121: note = B43 VH
QVQLLESGAELVRPGSSVKISCKASGYAFSSYWMNWVKQRPGQGLEWIGQIWPGDGDTNYNGKFKGKATLTADESSSTAYMQLSSLRSEDSAVYSCARRETTTVGRYYYAMDYWGQGTTVT

SEQ ID NO: 4 - protein, 123 aa, synthetic construct
  REGION 1..123: note = HD37 VH
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVS

SEQ ID NO: 5 - protein, 120 aa, synthetic construct
  REGION 1..120: note = 4G7 VH
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVS

SEQ ID NO: 6 - protein, 119 aa, synthetic construct
  REGION 1..119: note = FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 7 - protein, 104 aa, synthetic construct
  REGION 1..104: note = RB4v1.0 VK
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIK

SEQ ID NO: 8 - protein, 104 aa, synthetic construct; identical to SEQ ID NO: 7
  REGION 1..104: note = RB4v1.2 VK

SEQ ID NO: 9 - protein, 111 aa, synthetic construct
  REGION 1..111: note = B43 VK
ELVLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 10 - protein, 111 aa, synthetic construct
  REGION 1..111: note = HD37 VK
DILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 11 - protein, 112 aa, synthetic construct
  REGION 1..112: note = 4G7 VK
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELK

SEQ ID NO: 12 - protein, 107 aa, synthetic construct
  REGION 1..107: note = FMC63 VK
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 13 - protein, 109 aa, synthetic construct
  REGION 1..109: note = Epratuzumab VH
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQG

SEQ ID NO: 14 - protein, 106 aa, synthetic construct
  REGION 1..106: note = Epratuzumab VL
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGQG

SEQ ID NO: 15 - protein, 445 aa, synthetic construct
  REGION 1..445: note = EMabC220-HC
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 219 aa, synthetic construct
  REGION 1..219: note = EMabC220-LC
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 17 - protein, 449 aa, synthetic construct
  REGION 1..449: note = RB4v1.2-HC
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 211 aa, synthetic construct
  REGION 1..211: note = RB4v1.2-LC
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19-109 - skipped (91 entries)

SEQ ID NO: 110 - protein, 329 aa, synthetic construct
  REGION 1..329: note = IgG1 HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111-113 - skipped (3 entries)

SEQ ID NO: 114 - protein, 329 aa, synthetic construct
  REGION 1..329: note = IgG1 HC constant region, BJ C->V
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115-149 - skipped (35 entries)

SEQ ID NO: 150 - protein, 105 aa, synthetic construct [sequence omitted]

SEQ ID NO: 151 - protein, 105 aa, synthetic construct
  REGION 1..105: note = kLC constant region, C105S
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 152-159 - skipped (8 entries)

SEQ ID NO: 160 - protein, 103 aa, synthetic construct
  REGION 1..103: note = lambdaLC constant region
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 161 - protein, 103 aa, synthetic construct
  REGION 1..103: note = lambdaLC constant region, C102S
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTESS

SEQ ID NO: 162-299 - skipped (138 entries)

SEQ ID NO: 300 - protein, 7 aa, synthetic construct
  REGION 1..7: note = AUNP12 peptide
FSESTNS

SEQ ID NO: 301 - protein, 7 aa, synthetic construct
  REGION 1..7: note = AUNP12 peptide
SNTSESF

SEQ ID NO: 302 - protein, 14 aa, synthetic construct
  REGION 1..14: note = AUNP12 peptide
FRVTQLAPKAQIKE

SEQ ID NO: 303 - protein, 22 aa, synthetic construct
  REGION 1..22: note = AUNP12 peptide
SNTSESFKFRVTQLAPKAQIKE

SEQ ID NO: 304-399 - skipped (96 entries)

SEQ ID NO: 400 - protein, 5 aa, synthetic construct
  REGION 1..5: note = BMS-936559/MDX-1105 Antibody 1 VH CDR1
DYGFS

SEQ ID NO: 401 - protein, 17 aa, synthetic construct
  REGION 1..17: note = BMS-936559/MDX-1105 Antibody 1 VH CDR2
WITAYNGNTNYAQKLQG

SEQ ID NO: 402 - protein, 8 aa, synthetic construct
  REGION 1..8: note = BMS-936559/MDX-1105 Antibody 1 VH CDR3
DYFYGMDV

SEQ ID NO: 403 - protein, 11 aa, synthetic construct
  REGION 1..11: note = BMS-936559/MDX-1105 Antibody 1 VL CDR1
RASQSVSSYLV

SEQ ID NO: 404 - protein, 7 aa, synthetic construct
  REGION 1..7: note = BMS-936559/MDX-1105 Antibody 1 VL CDR2
DASNRAT

SEQ ID NO: 405 - protein, 9 aa, synthetic construct
  REGION 1..9: note = BMS-936559/MDX-1105 Antibody 1 VL CDR3
QQRSNWPRT

SEQ ID NO: 406 - protein, 5 aa, synthetic construct
  REGION 1..5: note = BMS-936559/MDX-1105 Antibody 2 VH CDR1
TYAIS

SEQ ID NO: 407 - protein, 17 aa, synthetic construct
  REGION 1..17: note = BMS-936559/MDX-1105 Antibody 2VH CDR2
GIIPIFGKAHYAQKFQG

SEQ ID NO: 408 - protein, 14 aa, synthetic construct
  REGION 1..14: note = BMS-936559/MDX-1105 Antibody 2VH CDR3
KFHFVSGSPFGMDV

SEQ ID NO: 409 - protein, 11 aa, synthetic construct
  REGION 1..11: note = BMS-936559/MDX-1105 Antibody 2 VL CDR1
RASQSVSSYLA

SEQ ID NO: 410 - protein, 7 aa, synthetic construct
  REGION 1..7: note = BMS-936559/MDX-1105 Antibody 2 VL CDR2
DASNRAT

SEQ ID NO: 411 - protein, 8 aa, synthetic construct
  REGION 1..8: note = BMS-936559/MDX-1105 Antibody 2 VL CDR3
QQRSNWPT

SEQ ID NO: 412 - protein, 5 aa, synthetic construct
  REGION 1..5: note = BMS-936559/MDX-1105 Antibody 3 VH CDR1
SYDVH

SEQ ID NO: 413 - protein, 17 aa, synthetic construct
  REGION 1..17: note = BMS-936559/MDX-1105 Antibody 3 VH CDR2
WLHADTGITKFSQKFQG

SEQ ID NO: 414 - protein, 9 aa, synthetic construct
  REGION 1..9: note = BMS-936559/MDX-1105 Antibody 3 VH CDR3
ERIQLWFDY

SEQ ID NO: 415 - protein, 11 aa, synthetic construct
  REGION 1..11: note = BMS-936559/MDX-1105 Antibody 3 VL CDR1
RASQGISSWLA

SEQ ID NO: 416 - protein, 7 aa, synthetic construct
  REGION 1..7: note = BMS-936559/MDX-1105 Antibody 3 VL CDR2
AASSLQS

SEQ ID NO: 417 - protein, 9 aa, synthetic construct
  REGION 1..9: note = BMS-936559/MDX-1105 Antibody 3 VL CDR3
QQYNSYPYT

SEQ ID NO: 418 - protein, 121 aa, synthetic construct
  REGION 1..121: note = durvalumab/MEDI4736 VH Sequence
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 419 - protein, 108 aa, synthetic construct
  REGION 1..108: note = durvalumab/MEDI4736 VL Sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 420-499 - skipped (80 entries)

SEQ ID NO: 500 - protein, 107 aa, synthetic construct
  REGION 1..107: note = TRX518 VL seuqence
EIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIK

SEQ ID NO: 501 - protein, 118 aa, synthetic construct
  REGION 1..118: note = TRX518 VH sequence
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVS

SEQ ID NO: 502 - protein, 118 aa, synthetic construct
  REGION 1..118: note = TRX518 VH sequence
QVTLRESGPALVKPTQTLTLTCTFSGFSLSTSGMGVGWIRQPPGKALEWLAHIWWDDDKYYQPSLKSRLTISKDTSKNQVVLTMTNMDPVDTATYYCARTRRYFPFAYWGQGTLVTVS

SEQ ID NO: 503-599 - skipped (97 entries)

SEQ ID NO: 600 - protein, 450 aa, synthetic construct
  REGION 1..450: note = MEDI0562 Heavy chain sequence
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 601 - protein, 214 aa, synthetic construct
  REGION 1..214: note = MEDI0562 Light Chain sequence
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 602 - protein, 402 aa, synthetic construct
  REGION 1..402: note = MEDI6383
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDQDKIEALSSKVQQLERSIGLKDLAMADLEQKVLEMEASTQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 603 - protein, 121 aa, synthetic construct
  REGION 1..121: note = Ox40mAb24 VH sequence
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSS

SEQ ID NO: 604 - protein, 107 aa, synthetic construct
  REGION 1..107: note = OX40mAb24 VL sequence
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIK

SEQ ID NO: 605 - protein, 5 aa, synthetic construct
  REGION 1..5: note = INCAGN1949 Antibody a VH CDR1
GSAMH

SEQ ID NO: 606 - protein, 19 aa, synthetic construct
  REGION 1..19: note = INCAGN1949 Antibody a VH CDR2
RIRSKANSYATAYAASVKG

SEQ ID NO: 607 - protein, 10 aa, synthetic construct
  REGION 1..10: note = INCAGN1949 Antibody a VH CDR3
GIYDSSGYDY

SEQ ID NO: 608 - protein, 16 aa, synthetic construct
  REGION 1..16: note = INCAGN1949 Antibody a VL CDR1
RSSQSLLHSNGYNYLD

SEQ ID NO: 609 - protein, 7 aa, synthetic construct
  REGION 1..7: note = INCAGN1949 Antibody a VL CDR2
LGSNRAS

SEQ ID NO: 610 - protein, 9 aa, synthetic construct
  REGION 1..9: note = INCAGN1949 Antibody a VL CDR3
MQALQTPLT

SEQ ID NO: 611 - protein, 121 aa, synthetic construct
  REGION 1..121: note = INCAGN1949 Antibody b VH sequence
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTSGIYDSSGYDYWGQGTLVTVSS

SEQ ID NO: 612 - protein, 112 aa, synthetic construct
  REGION 1..112: note = INCAGN1949 Antibody b VL sequence
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 613-699 - skipped (87 entries)

SEQ ID NO: 700 - protein, 167 aa, synthetic construct
  REGION 1..167: note = Tremelimumab VH sequence
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 701 - protein, 139 aa, synthetic construct
  REGION 1..139: note = Tremelimumab VL sequence
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 702-799 - skipped (98 entries)

SEQ ID NO: 800 - protein, 451 aa, synthetic construct
  REGION 1..451: note = Rituximab Heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 801 - protein, 213 aa, synthetic construct
  REGION 1..213: note = Rituximab Light Chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0149556 A1
(43) Pub. Date: May 18, 2023

(54) COMBINATION THERAPY

(71) Applicants: ADC Therapeutics SA, Epalinges (CH); Medimmune Limited, Cambridge (GB)

(72) Inventors: Jay Marshall FEINGOLD, Murray Hill, NJ (US); Patricius Hendrikus Cornelis VAN BERKEL, Epalinges (CH); Jens WUERTHNER, Epalinges (CH); John HARTLEY, London (GB); Francesca ZAMMARCHI, Epalinges (CH)

(21) Appl. No.: 17/819,185

(22) Filed: Aug. 11, 2022

Related U.S. Application Data
(62) Division of application No. 16/605,701, filed on Oct. 16, 2019, now abandoned.

(30) Foreign Application Priority Data
Apr. 20, 2017 (GB) 1706253.0
Apr. 20, 2017 (GB) 1706254.8
Apr. 20, 2017 (GB) 1706256.3
Apr. 20, 2017 (GB) 1706257.1
Apr. 20, 2017 (GB) 1706258.9
Apr. 20, 2017 (GB) 1706259.7
Apr. 20, 2017 (GB) 1706260.5
Apr. 20, 2017 (GB) 1706261.3
Feb. 23, 2018 (GB) 1802947.0
Apr. 5, 2018 (GB) 1805660.6

(51) Int. Cl. A61K 47/68; A61P 35/00

(52) U.S. Cl. CPC A61K 47/6849; A61K 47/6807; A61P 35/00

(57) ABSTRACT

The present disclosure relates to combination therapies for the treatment of pathological conditions, such as cancer. In particular, the present disclosure relates to combination therapies comprising treatment with an Antibody Drug Conjugate (ADC),a secondary agent, and optionally an anti-CD20 agent.

### CROSS-REFERENCE TO RELATED APPLICATIONS

This application claims the benefit of GB1706261.3, GB1706260.5, GB1706259.7, GB1706258.9, GB1706257.1, GB1706256.3, GB1706254.8, and GB1706253.0, all filed 20 Apr. 2017; GB1802947.0 filed 23 Feb. 2018; and GB1805660.6 filed 5 Apr. 2018.

### REFERENCE TO AN ELECTRONIC SEQUENCE LISTING

The content of the electronic sequence listing named 0233-0007US2SL having a size 238,236 bytes and date of creation of Aug. 9, 2022 is herein incorporated by reference in its entirety.

### FIELD

The present disclosure relates to combination therapies for the treatment of pathological conditions, such as cancer. In particular, the present disclosure relates to combination therapies comprising treatment with an Antibody Drug Conjugate (ADC), a secondary agent, and optionally an anti-CD20 agent.

### BACKGROUND

### Antibody Therapy

Antibody therapy has been established for the targeted treatment of subjects with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumour cells in the treatment of cancer, targets delivery of the drug moiety to tumours, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9): 1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

### Cd19

CD19 is a 95 kDa membrane receptor that is expressed early in B cell differentiation and continues to be expressed until the B cells are triggered to terminally differentiate (Pezzutto et al.(1987), J. Immunol 138:2793; Tedder et al (1994) Immunol

Today 15:437). The CD19 extracellular domain contains two C2-type immunoglobulin (IG)-like domains separated by a smaller potentially disulfide-linked domain. The CD19 cytoplasmic domain is structurally unique, but highly conserved between human, mouse, and guinea pig (Fujimoto et al., (1998) Semin Immunol.10:267). CD19 is part of a protein complex found on the cell surface of B-lymphocytes. The protein complex includes CD19, CD21 (complement receptor, type 2), CD81 (TAPA-1), and CD225 (Leu-13) (Fujimoto, supra).

CD19 is an important regulator of transmembrane signals in B cells. An increase or decrease in the cell surface density of CD19 affects B cell development and function, resulting in diseases such as autoimmunity or hypogammaglobulinemia. The CD19 complex potentiates the response of B cells to antigen in vivo through cross-linking of two separate signal transduction complexes found on B cell membranes. The two signal transduction complexes, associated with membrane IgM and CD19, activate phospholipase C (PLC) by different mechanisms. CD19 and B cell receptor cross-linking reduces the number of IgM molecules required to activate PLC. CD19 also functions as a specialized adapter protein for the amplification of Arc family kinases (Hasegawa et ah, (2001) J Immunol 167:3190).

CD19 binding has been shown to both enhance and inhibit B-cell activation and proliferation, depending on the amount of cross-linking that occurs (Tedder, 1994, Immunol. Today 15:437). CD19 is expressed on greater than 90% of B-cell lymphomas and has been predicted to affect growth of lymphomas in vitro and in vivo.

### Therapeutic Uses of Anti-CD19 ADCs

The efficacy of an Antibody Drug Conjugate comprising an anti-CD19 antibody (an anti-CD19-ADC) in the treatment of, for example, cancer has been established - see, for example, WO2014/057117 and WO2016/166298.

Research continues to further improve the efficacy, tolerability, and clinical utility of anti-CD19 ADCs. To this end, the present authors have identified clinically advantageous combination therapies in which an anti-CD19 ADC is administered in combination with at least one secondary agent.

### Cd22

CD22 is a 135-kDa type I transmembrane sialoglycoprotein of the immunoglobulin (Ig) superfamily. CD22 expression is specific to B cells and is developmentally regulated so that expression is limited in pro-B and pre-B cells (Dorner & Goldenberg, 2007, Ther Clin Risk Manag 3:954-59). As B-cells mature, expression increases and localization of CD22 shifts to the cell surface (Dorner & Goldenberg, 2007). CD22 is strongly expressed on follicular, mantle and marginal-zone B cells, but is weakly present in germinal B cells (Dorner & Goldenberg, 2007). CD22 is an inhibitory co-receptor that down modulates B-cell receptor (BCR) signalling by setting a signalling threshold that prevents overstimulation of B cells (Nitschke, 2005, Curr Opin Immunol 17:290-97).

Antibodies against CD22, such as epratuzumab (hLL2), have been used for treatment of a variety of cancers and autoimmune diseases, including but not limited to acute lymphoblastic leukemia (Hoelzer et al., 2013, Curr Opin Oncol 25:701-6), chronic lymphocytic leukemia (Macromatis & Cheson, 2004, Blood Rev 18:137-48), non-Hodgkin’s lymphoma (Leonard et al., 2004, Clin Cancer Res 10:5327-34; Dorner & Goldenberg, 2007), follicular lymphoma (Illidge & Morchhauser, 2011, Best Pract Res Clin Haematol 24:279-93), diffuse large B-cell lymphoma (Micallef et al., 2011, Blood 118:4053-61), mantle cell lymphoma (Sharkey et al., 2012, Mol Cancer Ther 11:224-34), systemic lupus erythematosus (Dorner & Goldenberg, 2007; Strand et al., 2013, Rheumatology Nov. 22, 2013 Epub ahead of print; Wallace & Goldenberg, 2013, Lupus 22:400-5; Wallace et al., 2013, Rheumatology 52:1313-22; Wallace et al., 2014, Ann Rheum Dis 73:183-90), and primary Sjogren’s syndrome (Steinfeld et al., 2006, Arthritis Res Ther 8:R129; Dorner & Goldenberg, 2007). A phase III clinical trial of epratuzumab in systemic lupus erythematosus is currently in progress (see, e.g., ClinicalTrials.gov, “Study of Epratuzumab versus Placebo in Subjects with Moderate to Severe General Systemic Lupus Erythematosus (EMBODY 1)”). Because CD22 regulates B-cell functions and survival, it is an important link for modulating humoral immunity and proliferation of B-cell lymphomas and a target for therapeutic antibodies in cancer and autoimmune disease (Dorner & Goldenberg, 2007)

### Therapeutic Uses of Anti-CD22 ADCs

The efficacy of an Antibody Drug Conjugate comprising an anti-CD22 antibody (an anti-CD22-ADC) in the treatment of, for example, cancer has been established - see, for example, WO2014/057122 and WO2016/166307, or as described in Kantarjian et al., (2016, New Eng J Med).

Research continues to further improve the efficacy, tolerability, and clinical utility of anti-CD22 ADCs. To this end, the present authors have identified clinically advantageous combination therapies in which an anti-CD22 ADC is administered in combination with at least one secondary agent.

### SUMMARY

The present authors have further determined that administration of a combination of an ADC and a secondary agent to an individual that has either been treated with, or is being treated with, and anti-CD20 agent leads to a synergistic increase in treatment efficacy.

In cases an ADC is administered in combination with anti-CD20 agent as a secondary agent. That is, it is envisaged that a combination of [ADC + anti-CD20 agent] is administered to the individual in combination.

In some cases, an ADC is administered in combination with the anti-CD20 agent as a tertiary agent, in further combination with a secondary agent as described herein (such as a Bruton’s Tyrosine Kinase inhibitor (BTKi), a PD1 antagonist, a PD-L1 antagonist, a GITR agonist, an OX40 agonist, a CTLA-4 antagonist, Fludarabine or Cytarabine, a hypomethylating agent, or an agent that upregulates HER2 expression). That is, it is envisaged that a combination of [ADC + secondary agent + anti-CD20 agent] is administered to the individual in combination.

Accordingly, in a first aspect the present disclosure provides a method of selecting an individual as suitable for treatment with a combination of an ADC and a secondary agent, wherein the individual is selected for treatment with the combination of an ADC and a secondary agent if the individual has been treated, or is being treated, with an anti-CD20 agent. The individual may be selected for treatment if the individual is refractory to treatment, or further treatment, with the anti-CD20 agent.

In another aspect, the present disclosure provides a method for treating a disorder in an individual, the method comprising selecting an individual as suitable for treatment by a method of the first aspect, and then administering to the individual an effective amount of a combination of an ADC and a secondary agent. The method of treatment may further comprise administering an anti-CD20 agent in combination with the combination of an ADC and a secondary agent.

The present authors have determined that the administration of a combination of an ADC, a secondary agent, and optionally an anti-CD20 agent to an individual leads to unexpected clinical advantages.

In another aspect, the disclosure provides a method for treating a disorder in an individual, the method comprising administering to the individual an effective amount of an ADC, a secondary agent, and optionally an anti-CD20 agent. The individual may be selected for treatment according to a method according of the first aspect.

The disorder may be a proliferative disease, for example a cancer such as non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL).

The ADC may be anti-CD19-ADC, such as ADCX19 described herein.

The ADC may be anti-CD22-ADC, such as ADCX22 described herein.

The secondary agent may be a Bruton’s Tyrosine Kinase inhibitor (BTKi), a PD1 antagonist, a PD-L1 antagonist, a GITR agonist, an OX40 agonist, a CTLA-4 antagonist, Fludarabine or Cytarabine, a hypomethylating agent, an agent that upregulates HER2 expression, or an anti-CD20 agent.

The proliferative disease may be characterised by the presence of a neoplasm comprising both CD19+ve and CD19-ve cells. The proliferative disease may be characterised by the presence of a neoplasm comprising both CD22+ve and C22-ve cells.

The proliferative disease may be characterised by the presence of a neoplasm composed of CD19-ve neoplastic cells, optionally wherein the CD19-ve neoplastic cells are associated with CD19+ve neoplastic or non-neoplastic cells. The proliferative disease may be characterised by the presence of a neoplasm composed of CD22-ve neoplastic cells, optionally wherein the CD22-ve neoplastic cells are associated with CD22+ve neoplastic or non-neoplastic cells.

The individual may be human. The individual may have cancer, or may have been determined to have cancer. The individual may have, or have been determined to have, a CD19+ cancer or CD19+ tumour-associated non-tumour cells, such as CD19+ infiltrating B-cells. The individual may have, or have been determined to have, a CD22+ cancer or CD22+ tumour-associated non-tumour cells, such as CD22+ infiltrating B-cells.

The target cancer or cancer cells may be all or part of a solid tumour.

“Solid tumor” herein will be understood to include solid haematological cancers such as lymphomas (Hodgkin’s lymphoma or non-Hodgkin’s lymphoma) which are discussed in more detail herein.

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 Jan;27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

In the disclosed methods the ADC may be administered before the secondary agent, simultaneous with the secondary agent, or after the secondary agent. The ADC and secondary agent may be administered before the anti-CD20 agent, simultaneous with the anti-CD20 agent, or after the anti-CD20 agent. The disclosed methods may comprise administering a further chemotherapeutic agent to the individual.

In one aspect, the present disclosure provides an anti-CD20 agent, or a composition comprising an anti-CD20 agent, for use in a method of treatment as described herein.

In a further aspect, the present disclosure provides for the use of an anti-CD20 agent in the manufacture of a medicament for treating a disorder in an individual, wherein the treatment comprises a method of treatment as described herein.

In another aspect, the disclosure provides a first composition comprising an ADC for use in a method of treating a disorder in an individual, wherein the treatment comprises administration of the first composition in combination with a second composition comprising a secondary agent and, optionally, in combination with a third composition comprising an anti-CD20 agent.

Also provided by this aspect is a first composition comprising a secondary agent for use in a method of treating a disorder in an individual, wherein the treatment comprises administration of the first composition in combination with a second composition comprising an ADC and, optionally, in combination with a third composition comprising an anti-CD20 agent.

The disorder may be a proliferative disease, for example a cancer such as non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL).

The target cancer or cancer cells may be all or part of a solid tumour.

“Solid tumor” herein will be understood to include solid haematological cancers such as lymphomas (Hodgkin’s lymphoma or non-Hodgkin’s lymphoma) which are discussed in more detail herein.

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 Jan;27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The ADC may be anti-CD19-ADC, such as ADCX19 described herein.

The ADC may be anti-CD22-ADC, such as ADCX22 described herein.

The secondary agent may be a Bruton’s Tyrosine Kinase inhibitor (BTKi), a PD1 antagonist, a PD-L1 antagonist, a GITR agonist, an OX40 agonist, a CTLA-4 antagonist, Fludarabine or Cytarabine, a hypomethylating agent, an agent that upregulates HER2 expression, or an anti-CD20 agent.

The proliferative disease may be characterised by the presence of a neoplasm comprising both CD19+ve and CD19-ve cells. The proliferative disease may be characterised by the presence of a neoplasm comprising both CD22+ve and C22-ve cells.

The proliferative disease may be characterised by the presence of a neoplasm composed of CD19-ve neoplastic cells, optionally wherein the CD19-ve neoplastic cells are associated with CD19+ve neoplastic or non-neoplastic cells. The proliferative disease may be characterised by the presence of a neoplasm composed of CD22-ve neoplastic cells, optionally wherein the CD22-ve neoplastic cells are associated with CD22+ve neoplastic or non-neoplastic cells.

The individual may be human. The individual may have cancer, or may have been determined to have cancer. The individual may have, or have been determined to have, a CD19+ cancer or CD19+ tumour-associated non-tumour cells, such as CD19+ infiltrating B-cells. The individual may have, or have been determined to have, a CD22+ cancer or CD22+ tumour-associated non-tumour cells, such as CD22+ infiltrating B-cells.

The first composition may be administered before the second composition, simultaneous with the second composition, or after the second composition. The ADC and secondary agent may be administered before the anti-CD20 agent, simultaneous with the anti-CD20 agent, or after the anti-CD20 agent. The treatment may comprise administering a further chemotherapeutic agent to the individual.

In a further aspect, the disclosure provides the use of an ADC in the manufacture of a medicament for treating a disorder in an individual, wherein the medicament comprises an ADC, and wherein the treatment comprises administration of the medicament in combination with a composition comprising secondary agent and, optionally, in combination with a third composition comprising an anti-CD20 agent.

Also provided by this aspect is the use of secondary agent in the manufacture of a medicament for treating a disorder in an individual, wherein the medicament comprises a secondary agent, and wherein the treatment comprises administration of the medicament in combination with a composition comprising an ADC and, optionally, in combination with a third composition comprising an anti-CD20 agent.

The disorder may be a proliferative disease, for example a cancer such as non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL).

The target cancer or cancer cells may be all or part of a solid tumour.

“Solid tumor” herein will be understood to include solid haematological cancers such as lymphomas (Hodgkin’s lymphoma or non-Hodgkin’s lymphoma) which are discussed in more detail herein.

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 Jan;27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The ADC may be anti-CD19-ADC, such as ADCX19 described herein.

The ADC may be anti-CD22-ADC, such as ADCX22 described herein.

The secondary agent may be a Bruton’s Tyrosine Kinase inhibitor (BTKi), a PD1 antagonist, a PD-L1 antagonist, a GITR agonist, an OX40 agonist, a CTLA-4 antagonist, Fludarabine or Cytarabine, a hypomethylating agent, an agent that upregulates HER2 expression, or an anti-CD20 agent.

The proliferative disease may be characterised by the presence of a neoplasm comprising both CD19+ve and CD19-ve cells. The proliferative disease may be characterised by the presence of a neoplasm comprising both CD22+ve and C22-ve cells.

The proliferative disease may be characterised by the presence of a neoplasm composed of CD19-ve neoplastic cells, optionally wherein the CD19-ve neoplastic cells are associated with CD19+ve neoplastic or non-neoplastic cells. The proliferative disease may be characterised by the presence of a neoplasm composed of CD22-ve neoplastic cells, optionally wherein the CD22-ve neoplastic cells are associated with CD22+ve neoplastic or non-neoplastic cells.

The individual may be human. The individual may have cancer, or may have been determined to have cancer. The individual may have, or have been determined to have, a CD19+ cancer or CD19+ tumour-associated non-tumour cells, such as CD19+ infiltrating B-cells. The individual may have, or have been determined to have, a CD22+ cancer or CD22+ tumour-associated non-tumour cells, such as CD22+ infiltrating B-cells.

The medicament may be administered before the composition, simultaneous with the composition, or after the composition. The ADC and secondary agent may be administered before the anti-CD20 agent, simultaneous with the anti-CD20 agent, or after the anti-CD20 agent. The treatment may comprise administering a further chemotherapeutic agent to the individual.

Another aspect of the disclosure provides a kit comprising:
a first medicament comprising an ADC;
a second medicament comprising a secondary agent; and, optionally,
   (i) a third medicament comprising an anti-CD20 agent, and/or
   (ii) a package insert comprising instructions for administration of the first medicament to an individual in combination with the second medicament and, optionally, in further combination with the third medicament, if present, for the treatment of a disorder.

Also provided by this aspect is a kit comprising a medicament comprising an ADC and a package insert comprising instructions for administration of the medicament to an individual in combination with a composition comprising a secondary agent and, optionally, in further combination with an anti-CD20 agent, for the treatment of a disorder.

Further provided by this aspect is a kit comprising a medicament comprising a secondary agent and a package insert comprising instructions for administration of the medicament to an individual in combination with a composition comprising an ADC and, optionally, in further combination with an anti-CD20 agent, for the treatment of a disorder.

The disorder may be a proliferative disease, for example a cancer such as non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL).

The target cancer or cancer cells may be all or part of a solid tumour.

“Solid tumor” herein will be understood to include solid haematological cancers such as lymphomas (Hodgkin’s lymphoma or non-Hodgkin’s lymphoma) which are discussed in more detail herein.

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 Jan;27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The ADC may be anti-CD19-ADC, such as ADCX19 described herein.

The ADC may be anti-CD22-ADC, such as ADCX22 described herein.

The secondary agent may be a Bruton’s Tyrosine Kinase inhibitor (BTKi), a PD1 antagonist, a PD-L1 antagonist, a GITR agonist, an OX40 agonist, a CTLA-4 antagonist, Fludarabine or Cytarabine, a hypomethylating agent, an agent that upregulates HER2 expression, or an anti-CD20 agent.

The proliferative disease may be characterised by the presence of a neoplasm comprising both CD19+ve and CD19-ve cells. The proliferative disease may be characterised by the presence of a neoplasm comprising both CD22+ve and C22-ve cells.

The proliferative disease may be characterised by the presence of a neoplasm composed of CD19-ve neoplastic cells, optionally wherein the CD19-ve neoplastic cells are associated with CD19+ve neoplastic or non-neoplastic cells. The proliferative disease may be characterised by the presence of a neoplasm composed of CD22-ve neoplastic cells, optionally wherein the CD22-ve neoplastic cells are associated with CD22+ve neoplastic or non-neoplastic cells.

The individual may be human. The individual may have cancer, or may have been determined to have cancer. The individual may have, or have been determined to have, a CD19+ cancer or CD19+ tumour-associated non-tumour cells, such as CD19+ infiltrating B-cells. The individual may have, or have been determined to have, a CD22+ cancer or CD22+ tumour-associated non-tumour cells, such as CD22+ infiltrating B-cells.

The medicament or composition comprising the ADC may be administered before the medicament or composition comprising the secondary agent, simultaneous with the medicament or composition comprising the secondary agent, or after the medicament or composition comprising the secondary agent. The ADC and secondary agent may be administered before the anti-CD20 agent, simultaneous with the anti-CD20 agent, or after the anti-CD20 agent. The treatment may comprise administering a further chemotherapeutic agent to the individual.

In a yet further aspect, the disclosure provides a composition comprising an ADC, a secondary agent, and optionally an anti-CD20 agent.

Also provided in this aspect of the disclosure is a method of treating a disorder in an individual, the method comprising administering to the individual an effective amount of the composition comprising an ADC, a secondary agent, and optionally an anti-CD20 agent.

Also provided in this aspect of the disclosure is a composition comprising an ADC, a secondary agent, and optionally an anti-CD20 agent, for use in a method of treating a disorder in an individual.

Also provided in this aspect of the disclosure is the use of a composition comprising an ADC, a secondary agent, and optionally an anti-CD20 agent, in the manufacture of a medicament for treating a disorder in an individual.

Also provided in this aspect of the disclosure is a kit comprising composition comprising an ADC, a secondary agent, and optionally an anti-CD20 agent, and a set of instructions for administration of the medicament to an individual for the treatment of a disorder.

The disorder may be a proliferative disease, for example a cancer such as non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL).

The target cancer or cancer cells may be all or part of a solid tumour.

“Solid tumor” herein will be understood to include solid haematological cancers such as lymphomas (Hodgkin’s lymphoma or non-Hodgkin’s lymphoma) which are discussed in more detail herein.

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 Jan;27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The ADC may be anti-CD19-ADC, such as ADCX19 described herein.

The ADC may be anti-CD22-ADC, such as ADCX22 described herein.

The secondary agent may be a Bruton’s Tyrosine Kinase inhibitor (BTKi), a PD1 antagonist, a PD-L1 antagonist, a GITR agonist, an OX40 agonist, a CTLA-4 antagonist, Fludarabine or Cytarabine, a hypomethylating agent, an agent that upregulates HER2 expression, or an anti-CD20 agent.

The proliferative disease may be characterised by the presence of a neoplasm comprising both CD19+ve and CD19-ve cells. The proliferative disease may be characterised by the presence of a neoplasm comprising both CD22+ve and C22-ve cells.

The proliferative disease may be characterised by the presence of a neoplasm composed of CD19-ve neoplastic cells, optionally wherein the CD19-ve neoplastic cells are associated with CD19+ve neoplastic or non-neoplastic cells. The proliferative disease may be characterised by the presence of a neoplasm composed of CD22-ve neoplastic cells, optionally wherein the CD22-ve neoplastic cells are associated with CD22+ve neoplastic or non-neoplastic cells.

The individual may be human. The individual may have cancer, or may have been determined to have cancer. The individual may have, or have been determined to have, a CD19+ cancer or CD19+ tumour-associated non-tumour cells, such as CD19+ infiltrating B-cells. The individual may have, or have been determined to have, a CD22+ cancer or CD22+ tumour-associated non-tumour cells, such as CD22+ infiltrating B-cells.

The ADC and secondary agent may be administered before the anti-CD20 agent, simultaneous with the anti-CD20 agent, or after the anti-CD20 agent. The treatment may comprise administering a further chemotherapeutic agent to the individual.

### DETAILED DESCRIPTION

### Antibody Drug Conjugates (ADCs)

The present disclosure relates to the improved efficacy of combinations of an ADC and a secondary agent.

The ADC can deliver a drug to a target location. The target location is preferably a proliferative cell population. The antibody is an antibody for an antigen present on a proliferative cell population. In one aspect the antigen is absent or present at a reduced level in a non-proliferative cell population compared to the amount of antigen present in the proliferative cell population, for example a tumour cell population.

The ADC may comprise a linker which may be cleaved so as to release the drug at the target location. The drug may be a compound selected from RelA, RelB, RelC, RelD or RelE. Thus, the conjugate may be used to selectively provide a compound RelA, RelB, Rel C, RelD or RelE to the target location.

The linker may be cleaved by an enzyme present at the target location.

The disclosure particularly relates treatment with an anti-CD19 ADC disclosed in WO2014/057117, and as herein described.

The disclosure also particularly relates treatment with an anti-CD22 ADC disclosed in WO2014/057122, and as herein described.

### Anti-CD19 ADCs

As used herein, the term “CD19-ADC” refers to an ADC in which the antibody component is an anti-CD19 antibody. The term “PBD-ADC” refers to an ADC in which the drug component is a pyrrolobenzodiazepine (PBD) warhead. The term “anti-CD19-ADC” refers to an ADC in which the antibody component is an anti-CD19 antibody, and the drug component is a PBD warhead.

The ADC may comprise a conjugate of formula L - (D^{L})_{P}, where D^{L} is of formula | or ||:

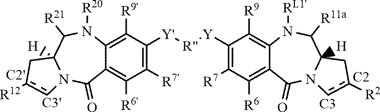

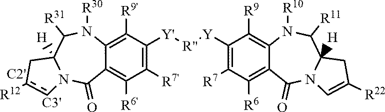

wherein:
L is an antibody (Ab) which is an antibody that binds to CD19;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id)

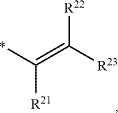

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms R¹² group is no more than 5;
(ie)

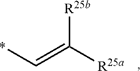

wherein one of R^{25a} and R^{25b} is H and the other is selected from: phenyl, which phenyl is optionally substituted by a group selected from halo, methyl, methoxy; pyridyl; and thiophenyl; and
(if)

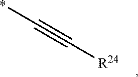

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,

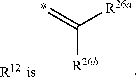

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R⁶′, R^{7’}, R⁹′ are selected from the same groups as R⁶, R⁷ and R⁹ respectively;

### Formula I

R^{L1}′ is a linker for connection to the antibody (Ab); R^{11a} is selected from OH, OR^{A}, where R^{A} is C₁₋₄ alkyl, and SO_{Z}M, where z is 2 or 3 and M is a monovalent pharmaceutically acceptable cation; R²⁰ and R²¹ either together form a double bond between the nitrogen and carbon atoms to which they are bound or; R²⁰ is selected from H and R^{c}, where R^{c} is a capping group; R²¹ is selected from OH, OR^{A} and SO_{z}M; when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C_{1-S} saturated aliphatic alkyl;
(ic) C₃₋₆ saturated cycloalkyl;
(id)

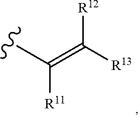

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;
(ie)

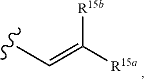

wherein one of R^{15a} and R^{15b} is H and the other is selected from: phenyl, which phenyl is optionally substituted by a group selected from halo, methyl, pyridyl; and thiophenyl; and
(if)

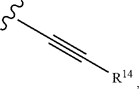

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is

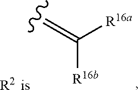

where R^{16a} and R^{16b} are independently selected from H, F, C_{1–4} saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

### Formula II

R²² is of formula Illa, formula lllb or formula lllc:

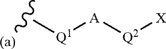

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;
(b)

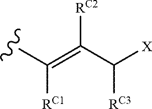

where;
R^{C1}, R^{C2} and R^{C3} are independently selected from H and unsubstituted C₁₋₂ alkyl;
(c)

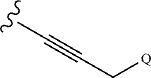

where Q is selected from O-R^{L2}’, S-R^{L2}′ and NR^{N}-R^{L2}’, and R^{N} is selected from H, methyl and ethyl
X is selected from the group comprising: O-R^{L2}’, S-R^{L2}′, CO₂-R^{L2′}, CO-R ^{L2}′ , NH-C(=O)-R^{L2}’, NHNH-R^{L2}, CONHNH-R^{L2},

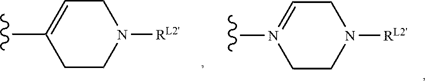

NR^{N}R^{L2’}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{Z}M;
R³⁰ and R³¹ either together form a double bond between the nitrogen and carbon atoms to which they are bound or;
R³⁰ is H and R³¹ is selected from OH, OR^{A} and SO_{Z}M.

In some embodiments L-R^{L1}′ or L-R^{L2}’ is a group:

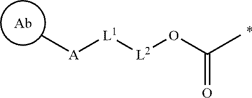

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.

In some of these embodiments, L¹ is enzyme cleavable.

It has previously been shown that such ADCs are useful in the treatment of CD19 expressing cancers (see, for example, WO2014/057117, which is incorporated by reference herein in its entirety).

The term anti-CD19-ADC may include any embodiment described in WO2014/057117. In particular, in preferred embodiments the ADC may have the chemical structure:

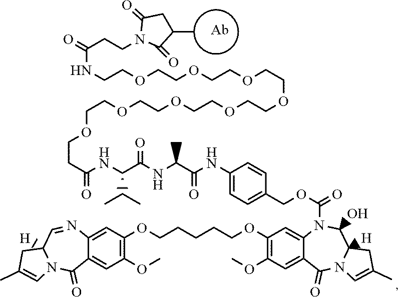

where the Ab is a CD19 antibody, and the DAR is between 1 and 8.

The antibody may comprise a VH domain having the sequence according to any one of SEQ ID NOs. 1, 2, 3, 4, 5 or 6, optionally further comprising a VL domain having the sequence according to any one of SEQ ID NOs. 7, 8, 9, 10, 11 or 12.

In some aspects the antibody component of the anti-CD19-ADC is an antibody comprising: VH and VL domains respectively having the sequences of: SEQ ID NO. 1 and SEQ ID NO. 7, SEQ ID NO. 2 and SEQ ID NO. 8, SEQ ID NO. 3 and SEQ ID NO. 9, SEQ ID NO. 4 and SEQ ID NO. 10, SEQ ID NO. 5 and SEQ ID NO. 11, or SEQ ID NO. 6 and SEQ ID NO. 12.

In preferred embodiments the antibody comprises a VH domain having the sequence according to SEQ ID NO. 2. In preferred embodiments the antibody comprises a VL domain having the sequence according to SEQ ID NO. 8.

In preferred embodiments the antibody comprises a VH domain and a VL domain, the VH and domain having the sequence of SEQ ID NO. 2 and the VL domain having the sequences of SEQ ID NO. 8.

The VH and VL domain(s) may pair so as to form an antibody antigen binding site that binds CD19.

In some embodiments the antibody is an intact antibody comprising a VH domain and a VL domain, the VH and VL domains having sequences of SEQ ID NO. 2 and SEQ ID NO. 8.

In some embodiments the antibody is an antibody comprising a heavy chain having sequences of SEQ ID NO. 17 and a light chain having the sequences of SEQ ID NO. 18.

In some embodiments the antibody is a fully human monoclonal IgG1 antibody, preferably IgG1,_{K}.

In some embodiments the antibody is the RB4v1.2 antibody described in WO2014/057117.

In an aspect the antibody is an antibody as described herein which has been modified (or further modified) as described below. In some embodiments the antibody is a humanised, deimmunised or resurfaced version of an antibody disclosed herein.

The most preferred anti-CD19-ADC for use with the aspects of the present disclosure is ADCX19, as described herein below.

### Adcx19

ADCX19 is an antibody drug conjugate composed of a humanized antibody against human CD19 attached to a pyrrolobenzodiazepine (PBD) warhead via a cleavable linker. The mechanism of action of ADCX19 depends on CD19 binding. The CD19 specific antibody targets the antibody drug conjugate (ADC) to cells expressing CD19. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period (Hartley 2011).

It has the chemical structure:

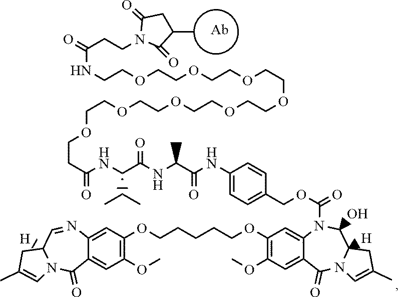

Ab represents Antibody RB4v1.2 (antibody with the VH and VL sequences SEQ ID NO. 2 and SEQ ID NO. 8, respectively). It is synthesised as described in WO2014/057117 (RB4v1.2-E) and typically has a DAR (Drug to Antibody Ratio) of 2 +/- 0.3..

CD19 binding

The “first target protein” (FTP) as used herein may be CD19.

As used herein, “binds CD19” is used to mean the antibody binds CD19 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD19 with an association constant (Kₐ) at least 2, 3, 4, 5, 10, 20, 50, 100, 200, 500, 1000, 2000, 5000, 10⁴, 10⁵ or 10⁶-fold higher than the antibody’s association constant for BSA, when measured at physiological conditions. The antibodies of the invention can bind CD19 with a high affinity. For example, in some embodiments the antibody can bind CD19 with a K_{D} equal to or less than about 10⁻⁶ M, such as 1 x 10⁻⁶, 10⁻⁷, 10⁻⁸, 10⁻⁹,10⁻¹⁰, 10⁻¹¹, 10⁻¹², 10-¹³ or 10⁻ 14

In some embodiments, CD19 polypeptide corresponds to Genbank accession no. NP_001171569, version no. NP_001171569.1 GI:296010921, record update date: Sep. 10, 2012 12:43 AM. In one embodiment, the nucleic acid encoding CD19 polypeptide corresponds to Genbank accession no NM_001178098, version no. NM_001178098.1 GI:296010920, record update date: Sep. 10, 2012 12:43 AM.In some embodiments, CD19 polypeptide corresponds to Uniprot/Swiss-Prot accession No. P15391.

### Anti-CD22 ADCs

As used herein, the term “CD22-ADC” refers to an ADC in which the antibody component is an anti-CD22 antibody. The term “PBD-ADC” refers to an ADC in which the drug component is a pyrrolobenzodiazepine (PBD) warhead. The term “anti-CD22-ADC” refers to an ADC in which the antibody component is an anti-CD22 antibody, and the drug component is a PBD warhead.

The ADC may comprise a conjugate of formula L - (D^{L})_{P}, where D^{L} is of formula l or ll:

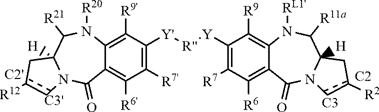

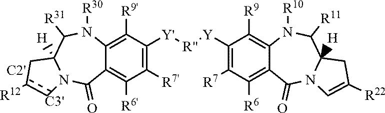

wherein:
L is an antibody (Ab) which is an antibody that binds to CD22;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id)

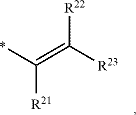

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;
(ie)

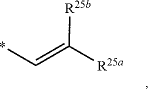

wherein one of R^{25a} and R^{25b} is H and the other is selected from: phenyl, which phenyl is optionally substituted by a group selected from halo, methyl, methoxy; pyridyl; and thiophenyl; and
(if)

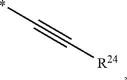

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,

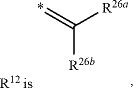

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6’}, R^{7’}, R^{9’} are selected from the same groups as R⁶, R⁷ and R⁹ respectively;

### Formula I

R^{L1’} is a linker for connection to the antibody (Ab);

_{R}^{11a} is selected from OH, OR^{A}, where R^{A} is C₁₋₄ alkyl, and SO_{Z}M, where z is 2 or 3 and M is a monovalent pharmaceutically acceptable cation;

R²⁰ and R²¹ either together form a double bond between the nitrogen and carbon atoms to which they are bound or;

R²⁰ is selected from H and R^{c}, where R^{c} is a capping group;

R²¹ is selected from OH, OR^{A} and SO_{z}M; when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id)

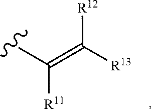

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;
(ie)

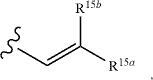

wherein one of R^{15a} and R^{15b} is H and the other is selected from: phenyl, which phenyl is optionally substituted by a group selected from halo, methyl, methoxy; pyridyl; and thiophenyl; and
(if)

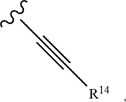

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,

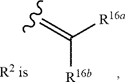

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

### Formula II

R²² is of formula Illa, formula lllb or formula lllc:
(a)

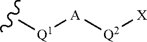

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;
(b)

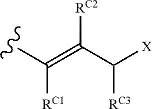

where;
R^{C1}, R^{C2} and R^{C3} are independently selected from H and unsubstituted C₁₋₂ alkyl;
(c)

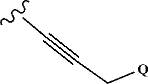

where Q is selected from O-R^{L2’}, S-R^{L2’} and NR^{N}-R^{L2’}, and R^{N} is selected from H, methyl and ethyl
X is selected from the group comprising: O-R^{L2’}, S-R^{L2’}, CO₂-R ^{L2’}, CO-R ^{L2’}, NH—C(═O)—R^{L2}′, NHNH-R^{L2}, CONHNH-R^{L2},

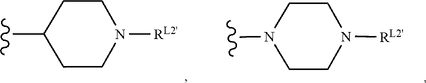

NR^{N}NR^{L2′}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{Z}M;
R³⁰ and R³¹ either together form a double bond between the nitrogen and carbon atoms to which they are bound or;
R³⁰ is H and R³¹ is selected from OH, OR^{A} and SO_{Z}M.

In some embodiments L-R^{L1}′ or L-R^{L2}’ is a group:

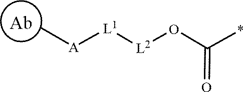

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.

In some of these embodiments, L¹ is enzyme cleavable.

It has previously been shown that such ADCs are useful in the treatment of CD22 expressing cancers (see, for example, WO2014/057122, which is incorporated by reference herein in its entirety).

The term anti-CD22-ADC may include any embodiment described in WO2014/057122. In particular, in preferred embodiments the ADC may have the chemical structure:

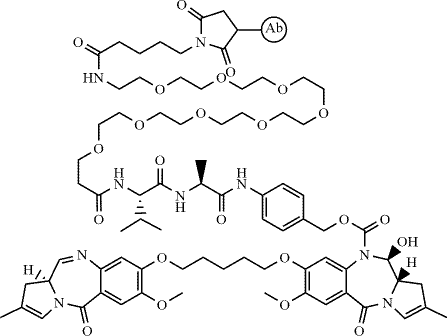

where the Ab is a CD22 antibody.

The antibody component of the anti-CD22 ADC

The antibody may comprise an amino acid substitution of an interchain cysteine residue by an amino acid that is not cysteine, wherein the conjugation of the drug moiety to the antibody is at an interchain cysteine residue

The antibody preferably comprises: (i) a heavy chain having an amino acid substitution of each of the interchain cysteine residues HC226 and HC229 according to the EU index as set forth in Kabat; (ii) a light chain having an amino acid substitution of the interchain cysteine residue _{K}LC214 or λLC213 according to the EU index as set forth in Kabat; and (iii) a heavy chain retaining the unsubstituted interchain cysteine HC220 according to the EU index as set forth in Kabat.

Preferably the drug moiety is conjugated to the unsubstituted interchain cysteine HC220. The interchain cysteine residues HC226 and HC229 may each be substituted for valine. The interchain cysteine residues _{K}LC214 or ALC213 may be substituted for serine.

In some embodiments, the antibody of the conjugates described herein comprises a light chain comprising the amino acid sequence of SEQ ID NO. 150, or fragment thereof, wherein the cysteine at position 105, if present, is substituted by an amino acid that is not cysteine. For example, SEQ ID NO. 151 discloses a light chain comprising the amino acid sequence of SEQ ID NO. 150 wherein the cysteine at position 105 is substituted by a serine residue.

In some embodiments, the antibody of the conjugates described herein comprises a light chain comprising the amino acid sequence of SEQ ID NO. 160, or fragment thereof, wherein the cysteine at position 102, if present, is substituted by an amino acid that is not cysteine. For example, SEQ ID NO. 161 discloses a light chain comprising the amino acid sequence of SEQ ID NO. 160 wherein the cysteine at position 102 is substituted by a serine residue.

In some embodiments the antibody comprises:
(i) a heavy chain having an amino acid substitution of each of the interchain cysteine residues HC226 and HC229 according to the EU index as set forth in Kabat, optionally wherein HC226 and HC229 is each substituted for valine;
(ii) a light chain having an amino acid substitution of the interchain cysteine residue _{K}LC214 or ALC213 according to the EU index as set forth in Kabat, optionally wherein _{K}LC214 or ALC213 is substituted for serine;
(iii) a heavy chain retaining the unsubstituted interchain cysteine HC220 according to the EU index as set forth in Kabat, optionally wherein the drug moiety is conjugated to the cysteine at HC220. In these embodiments, the antibody preferably further comprises a VH domain having the sequence according to SEQ ID NO. 13 and a VL domain having the sequence according to SEQ ID NO. 14. The light chain may comprise the amino acid sequence of: (i) SEQ ID NO. 150, or fragment thereof, wherein the cysteine at position 105, if present, is substituted by an amino acid that is not cysteine (such as in SEQ ID NO. 151); or SEQ ID NO. 160, or fragment thereof, wherein the cysteine at position 102, if present, is substituted by an amino acid that is not cysteine (such as in SEQ ID NO. 161).

The antibody may comprise a heavy chain comprising the amino acid sequence of SEQ ID NO. 110, and a light chain comprising the amino acid sequence of SEQ ID NO. 150 or SEQ ID NO. 160;
wherein each of the cysteines at positions 109 and 112 in SEQ ID NO: 110 is substituted by an amino acid that is not cysteine;
and wherein the cysteine at position 105 in SEQ ID NO: 150 or the cysteine at position 102 in SEQ ID NO: 160, is substituted by an amino acid that is not cysteine. Preferably the drug moiety is conjugated to the cysteine at position 103 of SEQ ID NO.110. In some embodiments the cysteines at positions 109 and 112 in SEQ ID NO: 110 are substituted for valine, such as in SEQ ID NO: 114. In some embodiments the cysteine at position 105 in SEQ ID NO: 150 or the cysteine at position 102 in SEQ ID NO: 160 is substituted by serine such as in SEQ ID NOs: 151 and 161.

In some aspects the antibody component of the anti-CD22-ADC is an antibody comprising: a VH domain having the sequence according to SEQ ID NO. 13.

The antibody may further comprise a VL domain having the sequence according to SEQ ID NO. 14.

In preferred embodiments the antibody comprises a VH domain having the sequence according to SEQ ID NO. 13 and a VL domain having the sequence according to SEQ ID NO. 14. For example, in some preferred embodiments, the antibody comprises:
a heavy chain having the sequence according to SEQ ID NO: 114;
a light chain having the sequence according to SEQ ID NO: 151;
a VH domain having the sequence according to SEQ ID NO. 13; and
a VL domain having the sequence according to SEQ ID NO. 14.

Preferably the drug moiety is conjugated to the cysteine at position 103 of SEQ ID NO.114.

In some embodiments the antibody is a fully human monoclonal IgG1 antibody, preferably IgG1,_{K}.

In some embodiments the antibody is the epratuzumab antibody described in WO2014/057122.

In come embodiments the antibody comprises a heavy chain having the sequence according to SEQ ID NO. 15 and a light chain having the sequence according to SEQ ID NO. 16. Preferably the drug moiety is conjugated to the cysteine at position 219 of SEQ ID NO.15.

In an aspect the antibody is an antibody as described herein which has been modified (or further modified) as described below. In some embodiments the antibody is a humanised, deimmunised or resurfaced version of an antibody disclosed herein.

The most preferred anti-CD22-ADC for use with the aspects of the present disclosure is ADCX22, as described herein below.

### Adcx22

ADCX22 is an antibody drug conjugate composed of a human antibody against human CD22 attached to a pyrrolobenzodiazepine (PBD) warhead via a cleavable linker. The mechanism of action of ADCX22 depends on CD22 binding. The CD22 specific antibody targets the antibody drug conjugate (ADC) to cells expressing CD22. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period (Hartley 2011).

It has the chemical structure:

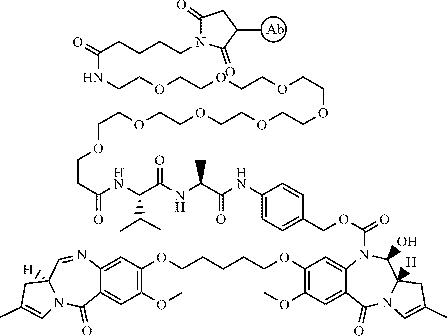

Ab represents Antibody EMabC220. This antibody comprises a heavy chain having the sequence according to SEQ ID NO. 15 and a light chain having the sequence according to SEQ ID NO. 16. Linkage to the drug occurs on Heavy Chain interchain cysteine Cys220 (EU numbering). HC220 corresponds to position 219 of SEQ ID NO.15.

It is noted that “having the sequence” has the same meaning as “comprising the sequence”; in particular, in some embodiments the heavy chain of ADCx22 is expressed with an additional terminal ‘K’ residue (so, ending ... SPGK), with the terminal K being optionally removed post-translationally to improve the homogeneity of the final therapeutic ADC product.

### CD22 Binding

The “first target protein” (FTP) as used herein may be CD22.

As used herein, “binds CD22” is used to mean the antibody binds CD22 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD22 with an association constant (Kₐ) at least 2, 3, 4, 5, 10, 20, 50, 100, 200, 500, 1000, 2000, 5000, 10⁴, 10⁵ or 10⁶-fold higher than the antibody’s association constant for BSA, when measured at physiological conditions. The antibodies of the invention can bind CD22 with a high affinity. For example, in some embodiments the antibody can bind CD22 with K_{D} equal to or less than about 10⁻⁶ M, such as 1 x 10⁻⁶, 10-⁷, 10-⁸, 10⁻⁹,10⁻¹⁰, 10⁻¹¹, 10⁻¹², 10⁻¹³ or 10⁻¹⁴.

In some embodiments, CD22 polypeptide corresponds to Genbank accession no. BAB15489, version no. BAB15489.1 Gl:10439338, record update date: Sep. 11, 2006 11:24 PM. In one embodiment, the nucleic acid encoding CD22 polypeptide corresponds to Genbank accession no AK026467, version no. AK026467.1 Gl:10439337, record update date: Sep. 11, 2006 11:24 PM.

### Secondary Agents

The recent development of agents that enhance anti-tumor immunity is rapidly changing the treatment of a broad range of cancers. However, these treatments are not effective in all cancer types responses are often not durable, and many patients receive little or no benefit from treatment. The prevailing assumption in the oncology field is that only combinations of immune-therapies with other treatment options will ultimately be able to cure cancer patients.

The ADC is well tolerated and active across a range of cancer types, and will likely be one component of combination therapies that increase the response rate and durability of treatment. The purpose of this disclosure is to combine the ADC with the secondary agent.

A secondary agent as described herein may be an Immune-oncology (lO) drug.

Immune-oncology (lO) drugs, a type of cancer therapy relying on the body’s immune system to help fight cancer, have shown enhanced durability of anti-tumor response. There are different types of IO, including but not limited to PD1 inhibitors, PD-L1 inhibitors, CLTL4 inhibitors, GITR agonists and OX40 agonists. Due to the considerable fraction of patients who are not cured by single agent immunotherapies and ultimately relapse, combination treatments with alternative IOdrugs or different therapeutic modalities are needed (see KS Peggs et al.2009, Clinical and Experimental Immunology, 157: 9-19 [doi:10.1111/j.1365-2249.2009.03912.x]; DM Pardoll 2012 [doi:10.1038/nrc3239]).

Immunogenic cell death (ICD) is a particular form of cell death that stimulates an immune response against dead-cell antigens (released by dying cells) and it is considered as one of the best way to induce an adaptive immune response and improve the efficacy of anticancer treatment. This process is frequently suboptimal, calling for combinatorial strategies that attempt to restore the full immunogenicity of cell death for therapeutic purposes. There are several anti-neoplastic agents that can induce ICD such as various anthracyclines (including doxorubicin, epirubicin and idarubicin), alkylating agents (including oxaliplatin and cyclophosphamide), the topoisomerase II inhibitor mitoxantrone, and the proteasomal inhibitor Bortezomib.

Antibody-drug conjugates, including those with a PBD warhead, may be particularly suited as combination partners because they are more targeted compared to conventional chemotherapy and expected to offer an increased antigen presentation to infiltrating cells as has been shown for auristatin-based ADCs.

Combining ADCs with IO therefore allows for dual benefits: on the one hand, the ADC will directly kill the tumor expressing the target, providing immediate anti-tumor activity, and on the other the immunogenic cell death induced by ADC mediated cell kill may boost a stronger and more durable adaptive immune response, as compared to when the IO is given as a single agent.

The secondary agent may be:
(a) a Bruton’s Tyrosine Kinase inhibitor (BTKi), such as Ibrutinib (Imbruvica), Acalabrutinib/ACP-196, ONO/GS-4059, Spebrutinib/AVL-292/CC-292, HM71224 (Poseltinib) or BGB-3111 (Zanubrutinib);
(b) a PD1 antagonist, such as pembrolizumab, nivolumab, MED10680, PDR001 (spartalizumab), Camrelizumab, AUNP12, Pidilizumab, Cemiplimab (REGN-2810), AMP-224, BGB-A317 (Tisleizumab), or BGB-108;
(c) a PD-L1 antagonist, such as atezolizumab (Tecentriq), BMS-936559/MDX-1105, durvalumab/MEDI4736, or MSB0010718C (Avelumab);
(d) a GITR (Glucocorticoid-Induced TNFR-Related protein) agonist, such as MEDI1873, TRX518, GWN323, MK-1248, MK-4166, BMS-986156 or INCAGN1876;
(e) an OX40 agonist, such as MEDI0562, MEDI6383, MOXR0916, RG7888, OX40mAb24, INCAGN1949, GSK3174998, or PF-04518600;
(f) a CTLA-4 antagonist, such as ipilimumab (brand name Yervoy) or Tremelimumab (Originally developed by Pfizer, now Medimmune);
(g) Fludarabine or Cytarabine;
(h) a hypomethylating agent, such as cytidine analogs - for example, 5-azacytidine (azacitidine) and 5-aza-2′-deoxycytidine (decitabine);
(i) an agent that upregulates HER2 expression, such as gemcitabine and tamoxifen; or
(j) an anti-CD20 agent, such as rituximab, obinutuzumab, Ibritumomab tiuxetan, tositumomab, Ofatumumab, Ocaratuzumab, Ocrelizumab, and Veltuzumab.

Each of these classes of secondary agent is described in more detail below.

### BTK Inhibitors

BTK is a non-receptor tyrosine kinase indispensable for B lymphocyte development, differentiation and signalling. Binding of antigen to the B-cell antigen receptor (BCR) triggers signalling that ultimately leads to B-cell activation. After BCR engagement and activation at the plasma membrane, BTK phosphorylates PLCG2 at several sites, igniting the downstream signaling pathway through calcium mobilization, followed by activation of the protein kinase C (PKC) family members. PLCG2 phosphorylation is performed in close cooperation with the adapter protein B-cell linker protein BLNK [Yang et al., Proc. Natl. Acad. Sci. U.S.A. 94:604-609(1997); Rodriguez et al., J. Biol. Chem. 276:47982-47992(2001)].

BTK acts as a platform to bring together a diverse array of signalling proteins and is implicated in cytokine receptor signalling pathways. It plays an important role in the function of immune cells of innate as well as in adaptive immunity, as a component of the Toll-like receptors (TLR) pathway. The TLR pathway acts as a primary surveillance system for the detection of pathogens and are crucial to the activation of host defence [Horwood et al. J. Immunol. 176:3635-3641(2006)].

Another key role for BTK is the regulation of TLR9 activation in splenic B-cells. Within the TLR pathway, BTK induces tyrosine phosphorylation of TIRAP which leads to TIRAP degradation.

BTK also plays also a critical role in transcription regulation as it is involved in the signalling pathway linking TLR8 and TLR9. As a result, BTK activity induces the activity of NF-kappa-B, which is itself involved in regulating the expression of hundreds of genes. Other transcriptional targets of BTK include ARID3A, NFAT and GTF2I BTK is required for the formation of functional ARID3A DNA-binding complexes; whilst BTK’s transient phosphorylatation of GTF21 causes it to translocate to the nucleus to bind regulatory enhancer elements to modulate gene expression [Rajaiya, Mol. Cell. Biol. 26:4758-4768(2006)].

BTK has a dual role in the regulation of apoptosis.

“BTK inhibitor” means any chemical compound or biological molecule that inhibits the activity of BTK. For example, agents that prevent kinase activity of BTK with an IC50 of 0.001 µM to about 2 µM.

The BTK enzyme inhibitory activity may measured, based on the protocol provided by the manufacturer, using Btk (Invitrogen Corporation) and the Z′-LYTE™ Kinase Assay Kit-Tyr1 peptide (Invitrogen Corporation), which contains the following reagents: Tyr-1 peptide, Thy-1 phosphopeptide, 5x kinase buffer, ATP, development reagent B, development buffer, and stop reagent. 5 µl/well of a solution of a BTK inhibitor may be diluted with dimethyl sulfoxide (DMSO), or DMSO, and 10 µl/well of the substrate/enzyme mixture solution dispensed to a 96- well assay plate and a reaction carried out for 20 minutes at 30° C. The substrate/enzyme mixture solution may be prepared by dilution with the kinase buffer (DL-dithiothreitol (DTT, 2.7 mM), 1.33x kinase buffer) to provide a final concentration for the Tyr- 1 peptide of 4 µM and a final BTK concentration of 5 nM. 5 µl/well of the adenosine triphosphate (ATP, final concentration = 36 µM) can then be added and a reaction carried out for 1 hour at 30° C. After the completion of the reaction, 10 µl., of a development solution, provided by diluting the development reagent B to 128x using the development buffer, may be added and a reaction carried out for an additional 1 hour at 30° C. The enzymatic reaction can then be stopped by adding 10 µl., of the stop solution. The fluorescence intensity at 445 nm and 520 nm in each well may be measured using a Fusion Universal Microplate Analyzer (PerkinElmer Inc.) fluorescence plate reader. The percent phosphorylation may be determined using the ratio of the emission at 445 nm (coumarin emission) to the emission at 520 nm (fluorescein emission) in accordance with the protocol provided with the kit.

The percent inhibition (%) by a BTK inhibitor may be calculated using the following equation. percent inhibition (%) of phosphorylation =1 - {(AC - AX)/(AC - AB)} x 100

AX : % phosphorylation when a BTK inhibitor has been added

AB : % phosphorylation in the absence of ATP addition (blank)

AC : % phosphorylation when only DMSO has been added (control)

The 50% inhibition value (IC50 value) for a BTK inhibitor may be determined from the inhibition curve based on the % inhibition at each concentration of a BTK inhibitor.

The BTKi Ibrutinib (Imbruvica) is a small molecule drug that covalently binds to Bruton’s tyrosine kinase (BTK) and has been used to treat B-cell cancers like mantle cell lymphoma, chronic lymphocytic leukemia, and Waldenström’s macroglobulinemia, a form of non-Hodgkin’s lymphoma.

Ibrutinib has been reported to reduce chronic lymphocytic leukemia (CLL) cell chemotaxis towards the chemokines CXCL12 and CXCL13, and inhibit cellular adhesion following stimulation at the B cell receptor (BCR) (S Ponader et al. 2011, doi:10.1182/blood-2011-10-386417. PMID 22180443.) Additionally, ibrutinib down-modulates the expression of CD20 by targeting the CXCR4/SDF1 axis (Pavlasova 2016, PMID 27480113. Together, these data are consistent with a mechanistic model whereby ibrutinib blocks BCR signalling, which drives B-cells into apoptosis and/or disrupts cell migration and adherence to protective tumour microenvironments.

In preclinical studies on chronic lymphocytic leukemia (CLL) cells, ibrutinib has been reported to promote apoptosis, inhibit proliferation, and also prevent CLL cells from responding to survival stimuli provided by the microenvironment (Pavlasova 2016). This also leads to a reduction of Mcl1 levels (anti-apoptotic protein) in malignant B cells. Treatment of activated CLL cells with ibrutinib resulted in inhibition of BTK tyrosine phosphorylation and also effectively abrogated downstream survival pathways activated by this kinase including ERK1/2, PI3K, and NF-κB. Additionally, ibrutinib inhibited proliferation of CLL cells in vitro, effectively blocking survival signals provided externally to CLL cells from the microenvironment including soluble factors (CD40L, BAFF, IL-6, IL-4, and TNF-α), fibronectin engagement and stromal cell contact.

Accordingly, combining an ADC, which targets a first target protein (FTP) with a BTKi is advantageous, because on the one hand, the ADC will directly kill the FTP positive tumor cells, while on the other hand the BTKi will interact with malignant B-cells resulting in inhibition of proliferation of the cancer cells. Next to FTP(+) tumor cells, FTP negative tumor cells in close proximity to FTP(+) tumor cells will potentially be killed by the bystander mechanism of the PBD-dimer released after cell kill of FTP(+) cells. Hence, the ADC will directly kill the tumor cells.

Furthermore, indications are that BTKi reduces tumour cell mobility and tips the regulatory balance in these cells more towards apoptosis. It is believed that these changes induced by the BTKi will make the tumour cells more susceptible to direct and indirect ADC medicated killing.

To show that ADCs works synergistically with BTKi, a panel of FTP (+) cell lines will be co-treated with a range of concentration of both ADC and BTK1. As negative controls, the same panel of cell lines will be treated with a range of concentrations of BTKi or with a range of concentration of ADC and vehicle. After incubation, two parameters will be measured: the amount of surface FTP (as determined by flow cytometry) and the in vitro cytotoxicity of the combinations (as determined by MTS assays). To determine the cytotoxicity, Cell viability is measured by adding MTS per well and incubating for 4 hours at 37° C. Percentage cell viability is calculated compared to the untreated control. Cytotoxic synergy is calculated by transforming the cell viability data into fraction affected, and calculating the combination index using the CalcuSyn analysis program.

BTKi suitable for use as secondary agents in the present disclosure include:
(1) 9-(1-acryloyl-3-azetidinyl)-6-amino-7-(4-phenoxyphenyl)-7, 9-dihydro-8H-purin-8-one,
(2) 6-amino-9-{(3R)-1-[(2E)-4-(dimethylamino)-2-butenoyl]-3-pyrrolidinyl}-7-(4-phenoxyphenyl)-7,9-dihydro-8H-purin-8-one,
(3) 9-[(1-acryloyl-4-piperidinyl)methyl]-6-amino-7-(4-phenoxyphenyl)-7,9-dihydro-8H-purin-8-one,
(4) 6-amino-9-[(3R)-1-(2-butynoyl)-3-pyrrolidinyl]-7-(4-phenoxyphenyl)-7,9-dihydro-8H-purin-8-one,
(5) 6-amino-9-{(3 S)-1-[(2E)-4-(dimethylamino)-2-butenoyl]-3-pyrrolidinyl}-7-(4-phenoxyphenyl)-7,9-dihydro-8H-purin-8-one,
(6) 6-amino-7-[4-(3-chlorophenoxy)phenyl]-9-{(3R)-1-[(2E)-4-(dimethylamino)-2-butenoyl]-3-pyrrolidinyl}-7 ,9-dihydro-8H-purin-8-one,
(7) 6-amino-9-[I-(2-butynoyl)-3-pyrrolidinyl]-7-(4-phenoxyphenyl)-7,9-dihydro-8H-purin-8-one, and
(8) 6-amino-9-{1-[(2E)-4-(dimethylamino)-2-butenoyl]-3-pyrrolidinyl}-7-(4-phenoxyphenyl)-7,9-dihydro-8H-purin-8-one.

Preferred BTK inhibitors for use as secondary agents in the present disclosure include (Ibrutnib being most preferred):
a) Ibrutinib (Imbruvica)
   i. CAS Number ➔936563-96-1 (see www.cas.org/content/chemical-substances/faqs)
   ii. NCBI Pubchem reference ➔24821094 (see pubchem.ncbi.nlm.nih.gov/)
   iii. IUPHAR/BPS reference ➔ 6912 (see www.guidetopharmacology.org/)
   iv. Unique Ingredient Identifier (UNII) ➔1X700SD4VX (see www.fda.gov/Forlndustry/DataStandards/SubstanceRegistrationSystem-UniquelngredientldentifierUNII/default.htm)

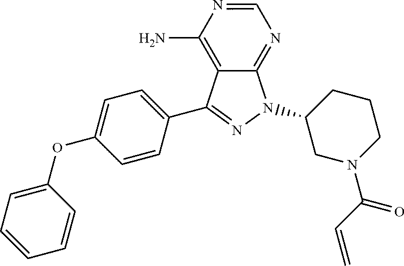 [SMILES: C=CC(=O)N1CCC[C@@H](n2nc(-c3ccc(Oc4ccccc4)cc3)c3c(N)ncnc32)C1]

Formula I, Ibrutinib: 1-[(3R)-3-[4-Amino-3-(4-phenoxyphenyl)-1H-pyrazolo[3,4-d]pyrimidin-1-yl]piperidin-1-yl]prop-2-en-1-one
b) Acalabrutinib/ACP-196
   i. CAS Number ➔1420477-60-6 (see www.cas.org/content/chemical-substances/faqs)
   ii. Chemspider ➔36764951 (see www.chemspider.com/)

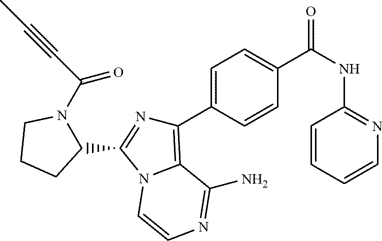

Formula II, Acalabrutinib: 4-{8-Amino-3-[(2S)-1-(2-butynoyl)-2-pyrrolidinyl]imidazo[1,5-a]pyrazin-1-yl}-N-(2-pyridinyl)benzamide
c) ONO/GS-4059
   i. CAS Number ➔1351635-67-0 (see www.cas.org/content/chemical-substances/fags)

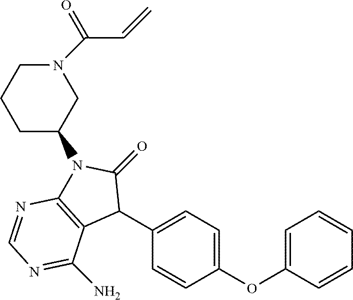 [SMILES: C=CC(=O)N1CCC[C@H](N2C(=O)C(c3ccc(Oc4ccccc4)cc3)c3c(N)ncnc32)C1]

Formula III, ONO/GS-4059: 6-amino-7,9-dihydro-9-[(3S)-1-(1-oxo-2-propen-1-yl)-3-piperidinyl]-7-(4-phenoxyphenyl)-8H-purin-8-one
d) Spebrutinib/AVL-292/CC-292
   i. CAS Number ➔1202757-89-8 (see www.cas.org/content/chemical-substances/faqs)
   ii. PubChem ID ➔59174488 (see pubchem.ncbi.nlm.nih.gov)

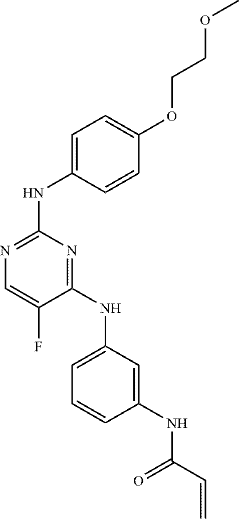 [SMILES: C=CC(=O)Nc1cccc(Nc2nc(Nc3ccc(OCCOC)cc3)ncc2F)c1]

Formula IV, Spebrutinib: N-[3-({5-fluoro-2-[4-(2-methoxyethoxy)anilino]pyrimidin-4-yl}amino)phenyl]prop-2-enamide
e) BGB-3111 (Zanubrutinib)
   i. CAS Number ➔1691249-45-2 (see www.cas.org/content/chemical-substances/faqs)

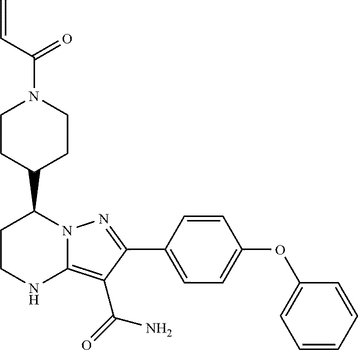 [SMILES: C=CC(=O)N1CCC([C@@H]2CCNc3c(C(N)=O)c(-c4ccc(Oc5ccccc5)cc4)nn32)CC1]

Formula V, Zanubrutinib: (7S)-4,5,6,7-Tetrahydro-7-[1-(1-oxo-2-propen-1-yl)-4-piperidinyl]-2-(4-phenoxyphenyl)pyrazolo[1,5-a]pyrimidine-3-carboxamide
f) HM71224 (Poseltinib)
   i. CAS Number ➔1353552-97-2 (see www.cas.org/content/chemical-substances/faqs)

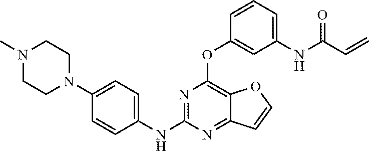 [SMILES: C=CC(=O)Nc1cccc(Oc2nc(Nc3ccc(N4CCN(C)CC4)cc3)nc3ccoc23)c1]

Formula VI, Poseltinib: N-(3-((2-((4-(4-methylpiperazin-1-yl)phenyl)amino)furo[3,2-d]pyrimidin-4-yl)oxy)phenyl)acrylamide

In some embodiments, BTK polypeptide corresponds to Genbank accession no. CAA41728, version no. CAA41728.1, record update date: Feb. 2, 2011 10:07 AM. In one embodiment, the nucleic acid encoding BTK polypeptide corresponds to Genbank accession no. X58957, version no. X58957.1, record update date: Feb. 2, 2011 10:07 AM. In some embodiments, BTK polypeptide corresponds to Uniprot/Swiss-Prot accession No. Q06187.

### PD1 Antagonists

Programmed death receptor I (PD1) is an immune-inhibitory receptor that is primarily expressed on activated T and B cells. Interaction with its ligands has been shown to attenuate T-cell responses both in vitro and in vivo. Blockade of the interaction between PD1 and one of its ligands, PD-L1, has been shown to enhance tumor-specific CD8+ T-cell immunity and may therefore be helpful in clearance of tumor cells by the immune system.

PD1 (encoded by the gene Pdcdl) is an Immunoglobulin superfamily member related to CD28, and CTLA-4. PD1 has been shown to negatively regulate antigen receptor signalling upon engagement of its ligands (PD-L1 and/or PD-L2). The structure of murine PD1 has been solved as well as the co-crystal structure of mouse PD1 with human PD-L1

(Zhang, X., et al., (2004) Immunity 20: 337-347; Lin, et al., (2008) Proc. Natl. Acad. Sci. USA 105: 301 1-6). PD1 and like family members are type I transmembrane glycoproteins containing an Ig Variable-type (V-type) domain responsible for ligand binding and a cytoplasmic tail that is responsible for the binding of signaling molecules. The cytoplasmic tail of PD1 contains two tyrosine-based signaling motifs, an ITIM (immunoreceptor tyrosine-based inhibition motif) and an ITSM (immunoreceptor tyrosine-based switch motif).

In humans, expression of PD1 (on tumor infiltrating lymphocytes) and/or PD-L1 (on tumor cells) has been found in a number of primary tumor biopsies assessed by immunohistochemistry. Such tissues include cancers of the lung, liver, ovary, cervix, skin, colon, glioma, bladder, breast, kidney, esophagus, stomach, oral squamous cell, urothelial cell, and pancreas as well as tumors of the head and neck (Brown, J. A., et al., (2003) J Immunol. I 70: 1257-1266; Dong H., et al., (2002) Nat. Med. 8: 793-800; Wintterle, et al., (2003) Cancer Res. 63: 7462-7467; Strome, S. E., et al., (2003) Cancer Res. 63: 6501-6505; Thompson, R.H., et al., (2006) Cancer Res. 66: 3381-5; Thompson, et al., (2007) Clin. Cancer Res. 13: 1 757-61; Nomi, T., et al., (2007) Clin. Cancer Res. 13: 2151-7). More strikingly, PD-ligand expression on tumor cells has been correlated to poor prognosis of cancer patients across multiple tumor types (reviewed in Okazaki and Honjo, (2007) Int. Immunol. 19: 813-824).

To date, numerous studies have shown that interaction of PD1 with its ligands (PD-L1 and PD-L2) leads to the inhibition of lymphocyte proliferation in vitro and in vivo. Blockade of the PD1/PD-L1 interaction could lead to enhanced tumor-specific T-cell immunity and therefore be helpful in clearance of tumor cells by the immune system. To address this issue, a number of studies were performed. In a murine model of aggressive pancreatic cancer (Nomi, T., et al. (2007) Clin. Cancer Res. 13: 2151-2157), the therapeutic efficacy of PD1/PD-L1 blockade was demonstrated. Administration of either PD1 or PD-L1 directed antibody significantly inhibited tumor growth. Antibody blockade effectively promoted tumor reactive CD8+ T cell infiltration into the tumor resulting in the up-regulation of anti-tumor effectors including IFN gamma, granzyme Band perforin. Additionally, the authors showed that PD1 blockade can be effectively combined with chemotherapy to yield a synergistic effect. In another study, using a model of squamous cell carcinoma in mice, antibody blockade of PD1 or PD-L1 significantly inhibited tumor growth (Tsushima, F., et al., (2006) Oral Oneal. 42: 268-274).

“PD1 antagonist” means any chemical compound or biological molecule that stimulates an immune reaction through inhibition of PD1 signalling.

To examine the extent of enhancement of, e.g., PD1 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) with PD1 inhibitors is advantageous, because on the one hand, the ADC will directly kill the FTP positive tumor cells, while on the other hand the PD1 inhibitor will engage the patient’s own immune system to eliminate the cancer cells. Next to FTP(+) tumor cells, FTP negative tumor cells in close proximity to FTP(+) tumor cells will potentially be killed by the bystander mechanism of the PBD-dimer released after cell kill of CD25(+) cells. Hence, the ADC will directly kill the tumor cells.

The resulting release of tumor associated antigens from cells that are killed with the PBD dimer will trigger the immune system, which will be further enhanced by the use of programmed cell death protein 1 (PD1) inhibitors, expressed on a large proportion of tumour infiltrating lymphocytes (TILs) from many different tumour types. Blockade of the PD1 pathway may enhance antitumour immune responses against the antigens released from the tumors killed by the ADC by diminishing the number and/or suppressive activity of intratumoral TReg cells.

The major function of PD1 is to limit the activity of T-cells at the time of an anti-inflammatory response to infection and to limit autoimmunity. PD1 expression is induced when T-cells become activated, and binding of one of its own ligands inhibits kinases involved in T-cell activation. Hence, in the tumor environment this may translate into a major immune resistance, because many tumours are highly infiltrated with TReg cells that probably further suppress effector immune responses. This resistance mechanism is alleviated by the use of PD1 inhibitors in combination with the ADC.

PD1 antagonists suitable for use as secondary agents in the present disclosure include:
a) a PD1 antagonist which inhibits the binding of PD1 to its ligand binding partners.
b) a PD1 antagonist which inhibits the binding of PD1 to PD- L1.
c) a PD1 antagonist which inhibits the binding of PD-1 to PDL2.
d) a PD1 antagonist which inhibits the binding of PD-1 to both PDLI and PDL2.
e) a PD1 antagonist of parts (a) to (d) which is an antibody.

Specific PD1 antagonists suitable for use as secondary agents in the present disclosure include:
a) pembrolizumab (brand name Keytruda)
   i. CAS Number ➔1374853-91-4
      (see www.cas.org/content/chemical-substances/faqs)
   ii. NCBI Pubchem reference ➔254741536
      (see pubchem.ncbi.nlm.nih.gov/)
   iii. DrugBank reference ➔DB09037
      (see www.drugbank.ca/)
   iv. Unique Ingredient Identifier (UNII) ➔DPT003T46P
      (see www.fda.gov/Forlndustry/DataStandards/SubstanceRegistrationSy stem-UniquelIgredientIdentifierUNII/default.htm)
b) nivolumab (brand name Opdivo)
   i. CAS Number ➔946414-94-4
      (see www.cas.org/content/chemical-substances/faqs)
   ii. DrugBank reference ➔DB09035
      (see www.drugbank.ca/)
c) MEDI0680 (formerly AMP-514)
   As described in WO2014/055648, WO2015/042246, WO2016/127052, WO2017/004016, WO2012/145493, US8609089, WO2016/007235, WO2016/011160; Int. J. Mol. Sci. 2016 Jul; 17(7): 1151, doi: 10.3390/ijms17071151; and Drug Discov Today, 2015 Sep;20(9):1127-34. doi: 10.1016/j.drudis.2015.07.003.
   See also clinical trials NCT02271945 and NCT02013804 at clinicaltrials.gov/ct2/home
d) PDR001 (spartalizumab)
   i. CAS Number ➔1935694-88-4
      (see www.cas.org/content/chemical-substances/faqs)
   ii. Unique Ingredient Identifier (UNII) ➔QOG25L6Z8Z
      (see www.fda.gov/Forlndustry/DataStandards/SubstanceRegistrationSy stem-UniqueIngredientIdentifierUNII/default.htm)
      As described in WO2016/007235 and WO2016/011160
      NCl thesaurus code ➔C121625 (see ncit.nci.nih.gov/ncitbrowser/)
e) Camrelizumab [INCSHR-1210] (Incyte)
   i. CAS Number ➔1798286-48-2
      (see www.cas.org/content/chemical-substances/faqs)
   ii. Unique Ingredient Identifier (UNII) ➔73096E137E
      (see www.fda.gov/Forlndustry/DataStandards/SubstanceRegistrationSy stem-UniqueIngredientIdentifierUNII/default.htm)
f) AUNP12 (peptide) (Aurigene/PierreFabre)
   i. Disclosed in WO2011/161699 as SEQ ID NO:49 a.k.a. “compound 8”, see Example 2 on page 77 of the A2 publication of WO2011/161699.
   ii. CAS Number ➔1353563-85-5
      (see www.cas.org/content/chemical-substances/faqs)
g) Pidilizumab (CT-01 1)
   i. CAS Number ➔1036730-42-3
      (see www.cas.org/content/chemical-substances/faqs)
   ii. Unique Ingredient Identifier (UNII) ➔B932PAQ1 BQ
      (see www.fdagov/ForIndustry/DataStandards/SubstanceRegistrationSy 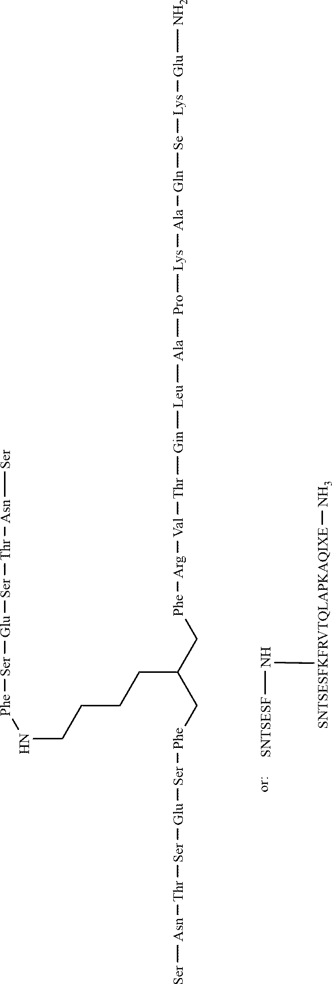
      stem-UniqueIngredientIdentifierUNII/default.htm)
h) Cemiplimab (formerly REGN-2810, SAR-439684)
   i. CAS Number ➔1801342-60-8
      (see www.cas.org/content/chemical-substances/faqs)
   ii. Unique Ingredient Identifier (UNII) ➔6QVL057INT
      (see www.fdagov/ForIndustry/DataStandards/SubstanceRegistrationSy stem-UniqueIngredientIdentifierUNII/default.htm)
      As described in WO2016/007235
      NCl thesaurus code ➔C121540
      (see ncit.nci.nih.gov/ncitbrowser/)
i) BGB-A317 (Tislelizumab)
   i. As described in US 9,834,606 B2
   ii. See clinical trial NCT03209973 ( clinicaltrials.gov/)
   iii. NCI thesaurus code C121775
      (see ncit.nci.nih.gov/ncitbrowser/)
j) BGB-108
   See WO2016/000619 and US8735553
k) AMP-224
   see clinical trial NCT02298946, clinicaltrials.gov/ct2/home

In some embodiments, PD1 polypeptide corresponds to Genbank accession no. AAC51773, version no. AAC51773.1, record update date: Jun. 23, 2010 09:24 AM. In one embodiment, the nucleic acid encoding PD1 polypeptide corresponds to Genbank accession no. U64863, version no. U64863.1, record update date: Jun. 23, 2010 09:24 AM. In some embodiments, PD1 polypeptide corresponds to Uniprot/Swiss-Prot accession No. Q15116.

### PD-L1 Antagonists

“PD-L1 antagonist” means any chemical compound or biological molecule that stimulates an immune reaction through inhibition of PD-L1 signalling.

To examine the extent of enhancement of, e.g., PD-L1 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with PD-L1 inhibitors is advantageous because, on the one hand, the ADC will directly kill the FTP positive tumor cells while, on the other hand, the PD-L1 inhibitor will engage the patient’s own immune system to eliminate the cancer cells.

Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor cells. The resulting release of tumor associated antigens from cells that are killed with the PBD dimer will trigger the immune system, which will be further enhanced by the use of programmed cell death protein 1 ligand inhibitors (PD-L1, aka B7-H1 or CD274).

PD-L1 is commonly upregulated on the tumour cell surface from many different human tumours. Interfering with the PD1 ligand expressed on the tumor will avoid the immune inhibition in the tumor microenvironment and therefore blockade of the PD1 pathway using PDL1 inhibitors may enhance antitumour immune responses against the antigens released from the tumors killed by the ADC.

Combining an ADC, which targets a first target protein (FTP) with PD1 inhibitors is advantageous, because on the one hand, the ADC will directly kill the FTP positive tumor cells, while on the other hand the PD1 inhibitor will engage the patient’s own immune system to eliminate the cancer cells. Next to FTP(+) tumor cells, FTP negative tumor cells in close proximity to FTP(+) tumor cells will potentially be killed by the bystander mechanism of the PBD-dimer released after cell kill of CD19(+) or CD22 (+) cells. Hence, the ADC will directly kill the tumor cells.

PD-L1 antagonists suitable for use as secondary agents in the present disclosure include PD-L1 antagonists that:
(a) are PD-L1 binding antagonists;
(b) inhibit the binding of PD-L1 to PD1;
(c) inhibit the binding of PD-L1 to B7-1;
(d) inhibit the binding of PD-L1 to both PD1 and B7-1;
(e) are anti-PD-L1 antibodies.

Specific PD-L1 antagonists suitable for use as secondary agents in the present disclosure include:
a) atezolizumab (MPDL3280A, brand name Tecentriq)
   i. CAS Number ➔1380723-44-3
      (see www.cas.org/content/chemical-substances/faqs)
   ii. DrugBank reference ➔DB11595
      (see www.drugbank.ca/)
   iii. Unique Ingredient Identifier (UNII) ➔52CMI0WC3Y
      (see www.fdagov/ForIndustry/DataStandards/SubstanceRegistrationSy stem-UniqueIngredientIdentifierUNII/default.htm)
b) BMS-936559 / MDX-1105
   I. CAS Number ➔1422185-22-5
      (see www.cas.org/content/chemical-substances/faqs)
   II. see clinical trial NCT02028403, clinicaltrials.gov/ct2/home
   III. See WO2007/005874 for antibody sequences, in particular the:
      i. Antibody having:
         a. VH CDR1 = DYGFS
         b. VH CDR2 = WITAYNGNTNYAQKLQG
         c. VH CDR3 = DYFYGMDV
         d. VL CDR1 = RASQSVSSYLV
         e. VL CDR2 = DASNRAT
         f. VL CDR3 =QQRSNWPRT
      ii. Antibody having:
         a. VH CDR1 = TYAIS
         b. VH CDR2 = GIIPIFGKAHYAQKFQG
         c. VH CDR3 = KFHFVSGSPFGMDV
         d. VL CDR1 = RASQSVSSYLA
         e. VL CDR2 = DASNRAT
         f. VL CDR3 =QQRSNWPT
      iii. Antibody having:
         a. VH CDR1 = SYDVH
         b. VH CDR2 = WLHADTGITKFSQKFQG
         c. VH CDR3 = ERIQLWFDY
         d. VL CDR1 = RASQGISSWLA
         e. VL CDR2 = AASSLQS
         f. VL CDR3 =QQYNSYPYT
c) durvalumab/MED14736
   i. CAS Number ➔1428935-60-7
      (see www.cas.org/content/chemical-substances/faqs)
   ii. Unique Ingredient Identifier (UNII) ➔28X28X90KV
      (see www.fdagov/ForIndustry/DataStandards/SubstanceRegistrationSy stem-niquelngredientldentifierUNII/default.htm)
   iii. VH sequence EVQLVESGGGLVQPGGSLRLSCAASGFTFSRYWMSWVRQAPGKGLEW VANIKQDGSEKYYVDSVKGRFTISRDNAKNSLYLQM NSLRAEDTAVYYC AREGGWFGELAFDYWGQGTLVTVSS
   iv. VL sequence EIVLTQSPGTLSLSPGERATLSCRASQRVSSSYLAWYQQKPGQAPRLLIY DASSRATGIPDRFSGSGSGTDFTLTISRLEPEDFAVYYCQQYGSLPWTF GQGTKVEIK
d) Avelumab / MSB0010718C
   i. CAS Number ➔1537032-82-8
      (see www.cas.org/content/chemical-substances/faqs)
   ii. Unique Ingredient Identifier (UNII) ➔KXG2PJ551I
      (see www.fdagov/ForIndustry/DataStandards/SubstanceRegistrationSy stem-niquelngredientldentifierUNII/default.htm)

In some embodiments, PD-L1 polypeptide corresponds to Genbank accession no. AAF25807, version no. AAF25807.1, record update date: Mar. 10, 2010 10:14 PM. In one embodiment, the nucleic acid encoding PD1 polypeptide corresponds to Genbank accession no. AF177937, version no. AF177937.1, record update date: Mar. 10, 2010 10:14 PM. In some embodiments, PD1 polypeptide corresponds to Uniprot/Swiss-Prot accession No. Q9NZQ7.

### GITR Agonists

The term “glucocorticoid-induced TNF receptor” (abbreviated herein as “GITR”), also known as TNF receptor superfamily 18 (TNFRSF18, CD357), TEASR, and 312C2, as used herein, refers to a member of the tumor necrosis factor/nerve growth factor receptor family. GITR is a 241 amino acid type I transmembrane protein characterized by three cysteine pseudo-repeats in the extracellular domain and specifically protects T-cell receptorinduced apoptosis, although it does not protect cells from other apoptotic signals, including Fas triggering, dexamethasone treatment, or UV irradiation (Nocentini, G., et al. (1997) Proc. Natl. Acad. Sci. USA 94:6216-622).

GITR activation increases resistance to tumors and viral infections, is involved in autoimmune/inflammatory processes and regulates leukocyte extravasation (Nocentini supra; Cuzzocrea, et al. (2004) J Leukoc. Biol. 76:933-940; Shevach, et al. (2006) Nat. Rev. Immunol. 6:613-618; Cuzzocrea, et al. (2006) J Immunol. I77:631-641; and Cuzzocrea, et al. (2007) FASEB J 21 :II 7-129). In tumor mouse models, agonist GITR antibody, DTA-1, was combined with an antagonist CTLA-4 antibody, and showed synergistic results in complete tumor regression of advanced stage tumors in some test group mice (Ko, et al. (2005) J Exp. Med. 7:885-891).

The nucleic acid and amino acid sequences of human GITR (hGITR), of which there are three splice variants, are known and can be found in, for example GenBank Accession Nos. gi:40354198, gi:23238190, gi:23238193, and gi:23238196.

“GITR agonist” means any chemical compound or biological molecule that stimulates an immune reaction through activation of GITR signalling. Also contemplated are soluble GITR-L proteins, a GITR binding partner.

To examine the extent of enhancement of, e.g., GITR activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with GITR agonists is advantageous, because on the one hand the ADC will directly kill the FTP positive tumor cells, while on the other hand the GITR agonist will engage the patient’s own immune system to eliminate the cancer cells. Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor. The resulting release of tumor associated antigens from cells killed with the PBD dimer will trigger the immune system, which will be further enhanced by the use of a GITR agonist.

GITR (Glucocorticoid-Induced TNFR-Related protein) is expressed transiently on activated T-cells and expressed constitutively at high levels on T-regs with further induction following activation. GITR ligation via its ligand GITRL stimulates both proliferation and function of both effector and regulatory CD4+ T cells. This promotes T-cell survival, and differentiation into effector cells, while abrogating suppression. Therefore it will be beneficial to target a FTP(+) tumor with the ADC, causing the antigenic cell death, while the GITR agonist induces a stronger, durable immune response.

Specific GITR agonists suitable for use as secondary agents in the present disclosure include:
a) MEDI1873, a GITR ligand fusion protein developed by Medlmmune
   See WO2016/196792, US20160304607
   NCl thesaurus code ➔C124651
      (see ncit.nci.nih.gov/ncitbrowser)
   See also clinical trial NCT023126110 at clinicaltrials.gov/ct2/home
   See Tigue NJ, Bamber L, Andrews J, et al. MEDI1873, a potent, stabilized hexameric agonist of human GITR with regulatory T-cell targeting potential.
   Oncoimmunology. 2017;6(3):e1280645.
   doi: 10.1080/2162402X.2017.1280645.
b) INCAGN1876, is an agonist antibody targeting the glucocorticoid-induced TNFR-related protein, or GITR. Discovered during a collaboration with Ludwig Cancer Research. INCAGN1876 is being co-developed with
   See clinical trials NCT02583165 and NCT03277352 at clinicaltrials.gov/ct2/home
c) TRX518, a humanized agylcosylated (Fc disabled) IgG1 anti-GITR mAb with immune-modulating activity developed by Leap Therapeutics
   See WO2006/105021 for sequences 58, 60-63; and EP2175884 sequences 1 - 7:
      VL comprising the sequence (CDR underline): EIVMTQSPATLSVSPGERATLSCKASQNVGTNVAWYQQKPG QAPRLLIYSASYRYSGIPARFSGSGSGTEFTLTISSLQSEDFA VYYCQQYNTDPLTFGGGTKVEIK
      VH comprising the sequence (CDR underline): QVTLRESGPALVKPTQTLTLTCTFSGFSLSTSGMGVGWI RQP PGKALEWLAHIWWDDDKYYNPSLKSRLTISKDTSKNQVVLTM TNM DPVDTATYYCARTRRYFPFAYWGQGTLVTVS QVTLRESGPALVKPTQTLTLTCTFSGFSLSTSGMGVGWI RQP PGKALEWLAHIWWDDDKYYQPSLKSRLTISKDTSKNQVVLTM TNM DPVDTATYYCARTRRYFPFAYWGQGTLVTVS
   See clinical trials NCT01239134 and NCT02628574 at
      clinicaltrials.gov/ct2/home
   NCl thesaurus code ➔C95023
   (see ncit.nci.nih.gov/ncitbrowser)
d) GWN323, an anti-GITR agonistic monoclonal antibody, which activates GITRs found on multiple types of T-cells. GWN323 is developed by Novartis
   See WO2016/196792
   NCl thesaurus code ➔C128028
      (see ncit.nci.nih.gov/ncitbrowser)
   See clinical trial NCT02740270 at clinicaltrials.gov/ct2/home
e) MK-1248, a humanized IgG4 anti-human glucocorticoid-induced tumor necrosis factor receptor (GITR) agonistic, monoclonal antibody (MoAb) with significantly reduced effector function
   See clinical trial NCT02553499 at clinicaltrials.gov/ct2/home
   MK-1248 has the same CDR as MK4166 (see Sukumar et al., Cancer Res. 2017)
f) MK-4166, a humanized IgG1 anti-human glucocorticoid-induced tumor necrosis factor receptor (GITR) agonistic monoclonal antibody (MoAb) with potential immunomodulating activity (see Sukumar et al., Cancer Res. 2017).
   See clinical trial NCT02132754 at clinicaltrials.gov/ct2/home
   See Sukumar, et al., (2017), Cancer Research. 77. canres.1439.2016. 10.1158/0008-5472. CA N-16-1439.
   NCl thesaurus code C116065
   (see ncit.nci.nih.gov/ncitbrowser/)
g) BMS-986156, An anti-human glucocorticoid-induced tumor necrosis factor receptor (GITR; tumor necrosis factor superfamily member 18; TNFRSF18; CD357) agonistic monoclonal antibody
   See clinical trial NCT02598960 at clinicaltrials.gov/ct2/home
   NCI thesaurus code C132267
      (see ncit.nci.nih.gov/ncitbrowser/)

Sequences of agonist anti-GITR antibodies are provided in WO2011/028683 and WO2006/105021.

In some embodiments, GITR polypeptide corresponds to Genbank accession no. AAD22635, version no. AAD22635.1, record update date: Mar. 10, 2010 09:42 PM. In one embodiment, the nucleic acid encoding GITR polypeptide corresponds to Genbank accession no. AF125304, version no. AF125304.1, record update date: Mar. 10, 2010 09:42 PM. In some embodiments, GITR polypeptide corresponds to Uniprot/Swiss-Prot accession No. Q9Y5U5.

### OX40 Agonists

OX40 (CD134; TNFRSF4) is a member of the TNFR super-family and is expressed by CD4 and CD8 T cells during antigen-specific priming. OX40 expression is largely transient following TCR/CD3 cross-linking, and by the presence of inflammatory cytokines. In the absence of activating signals, relatively few mature T cell subsets express OX40 at biologically relevant levels. Generating optimal “killer” CD8 T cell responses requires T cell receptor activation plus co-stimulation, which can be provided through ligation of OX40 using a OX40 agonist. This activating mechanism augments T cell differentiation and cytolytic function leading to enhanced anti-tumor immunity. Therefore it will be beneficial to target a FTP(+) tumor with the ADC, causing the antigenic cell death, while the OX40 agonist induces a stronger, durable immune response.

The OX40 agonist may be selected from the group consisting of an OX40 agonist antibody, an OX40L agonist fragment, an OX40 oligomeric receptor, and an OX40 immunoadhesin. In some embodiments, the OX40 binding agonist is a trimeric OX40L-Fc protein.

In some embodiments, the OX40 binding agonist is an OX40L agonist fragment comprising one or more extracellular domains of OX40L. In some embodiments, the OX40 binding agonist is an OX40 agonist antibody that binds human OX40. In some embodiments, the OX40 agonist antibody depletes cells that express human OX40. In some embodiments, the OX40 agonist antibody depletes cells that express human OX40 in vitro. In some embodiments, the cells are CD4+ effector T cells. In some embodiments, the cells are Treg cells. In some embodiments, the depleting is by ADCC and/or phagocytosis. In some embodiments, the depleting is by ADCC. In some embodiments, the OX40 agonist antibody binds human OX40 with an affinity of less than or equal to about 1 nM. In some embodiments, the OX40 agonist antibody increases CD4+ effector T cell proliferation and/or increasing cytokine production by the CD4+ effector T cell as compared to proliferation and/or cytokine production prior to treatment with anti-human OX40 agonist antibody. In some embodiments, the cytokine is gamma interferon. In some embodiments, the OX40 agonist antibody increases memory T cell proliferation and/or increasing cytokine production by the memory cell. In some embodiments, the cytokine is gamma interferon. In some embodiments, the OX40 agonist antibody inhibits Treg function. In some embodiments, the OX40 agonist antibody inhibits Treg suppression of effector T cell function. In some embodiments, effector T cell function is effector T cell proliferation and/or cytokine production. In some embodiments, the effector T cell is a CD4+ effector T cell. In some embodiments, the OX40 agonist antibody increases OX40 signal transduction in a target cell that expresses OX40. In some embodiments, OX40 signal transduction is detected by monitoring NFkB downstream signalling.

“OX40 agonist” means any chemical compound or biological molecule that stimulates an immune reaction through iactivation of OX40 signalling.

To examine the extent of enhancement of, e.g., OX40 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with OX40 agonists is advantageous, because on the one hand the ADC will directly kill the FTP positive tumor cells, while on the other hand the OX40 agonist will engage the patient’s own immune system to eliminate the cancer cells. Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor. The resulting release of tumor associated antigens from cells killed with the PBD dimer will trigger the immune system, which will be further enhanced by the use of a OX40 agonist.

Specific OX40 agonists suitable for use as secondary agents in the present disclosure include:
a) MEDI0562 (aka Tavolixizumab, Tavolimab)
   i. CAS Number ➔1635395-25-3
      (see www.cas.org/content/chemical-substances/faqs)
   ii. Unique Ingredient Identifier (UNII) ➔4LU9B48U4D
      (see www.fda./ForIndustry/DataStandards/SubstanceRegistrationSy stem-UniqueIngredientIdentifierUNII/default.htm)
   See clinical trial NCT02318394 at clinicaltrials.gov/ct2/home
   As described in WO2015/095423, WO2015/153514, WO2016/073380 & WO2016/081384
   NCl thesaurus code ➔C120041
      (see ncit.nci.nih.gov/ncitbrowser/)
   Heavy Chain sequence: QVQLQESGPGLVKPSQTLSLTCAVYGGSFSSGYWNWI▫RKH▫PGKGLEYIGYI ▫▫▫▫▫▫▫▫▫▫SYNGITYHNPSLKSRITINRDTSKNQYSLQLNSVTPEDTAVYYCARYKYDYDG ▫▫▫▫▫▫▫▫▫▫GHAMDYWGQGTLVTVSSASTKGPSVFPLAPSSKSTSGGTAALGCLVKDYFP ▫▫▫▫▫▫▫▫▫▫EPVTVSWNSGALTSGVHTFPAVLQSSGLYSLSSVVTVPSSSLGTQTYICNVN ▫▫▫▫▫▫▫▫▫▫HKPSNTKVDKRVEPKSCDKTHTCPPCPAPELLGGPSVFLFPPKPKDTLMISR ▫▫▫▫▫▫▫▫▫▫TPEVTCVVVDVSHEDPEVKFNWYVDGVEVHNAKTKPREEQYNSTYRVVSVL ▫▫▫▫▫▫▫▫▫▫TVLHQDWLNGKEYKCKVSNKALPAPIEKTISKAKGQPREPQVYTLPPSREEM ▫▫▫▫▫▫▫▫▫▫TKNQVSLTCLVKGFYPSDIAVEWESNGQPENNYKTTPPVLDSDGSFFLYSKL ▫▫▫▫▫▫▫▫▫▫TVDKSRWQQGNVFSCSVMHEALHNHYTQKSLSLSPG ▫▫▫▫▫▫ Light chain sequence: DIQMTQSPSSLSASVGDRVTITCRASQDISNYLNWYQQKPGKAPKLLIYYTSK ▫▫▫▫▫▫▫▫▫▫LHSGVPSRFSGSGSGTDYTLTISSLQPEDFATYYCQQGSALPWTFGQGTKV ▫▫▫▫▫▫▫▫▫▫AAPSVFIFPPSDEQLKSGTASVVCLLNNFYPREAKVQWKVDNALQS ▫▫▫▫▫▫▫▫▫▫GNSQESVTEQDSKDSTYSLSSTLTLSKADYEKHKVYACEVTHQGLSSPVTK ▫▫▫▫▫▫▫▫▫▫SFN▫RGEC
b) MEDl6383 (Efizonerimod alfa)
   i. CAS Number ➔1635395-27-5
      (see www.cas.org/content/chemical-substances/faqs)
   ii. Unique Ingredient Identifier (UNII) ➔1MH7C2X8KE
      (see www.fda./ForIndustry/DataStandards/SubstanceRegistrationSy stem-UniqueIngredientIdentifierUNII/default.htm)
   See clinical trial NCT02221960 at clinicaltrials.gov/ct2/home
   As described in WO2015/095423, WO2016/081384, and WO2016/189124
   NCI thesaurus code ➔C118282
      (see ncit.nci.nih.gov/ncitbrowser/)
   Amino acid sequence (Seq ID no.17 from WO2016/189124): ESKYGPPCPPCPAPEFLGGPSVFLFPPKPKDTLMISRTPEVTCVVVDVSQED ▫▫▫▫▫▫▫▫▫▫PEVQFNWYVDGVEVHNAKTKPREEQFNSTYRVVSVLTVLHQDWLNGKEYK ▫▫▫▫▫▫▫▫▫▫CKVSNKGLPSSIEKTISKAKGQPREPQVYTLPPSQEEMTKNQVSLTCLVKGF ▫▫▫▫▫▫▫▫▫▫YPSDIAVEWESNGQPENNYKTTPPVLDSDGSFFLYSRLTVDKSRWQEGNVF ▫▫▫▫▫▫▫▫▫▫SCSVMHEALHNHYTQKSLSLSLGKDQDKIEALSSKVQQLERSIGLKDLAMAD ▫▫▫▫▫▫▫▫▫▫LEQKVLEM▫EASTQVSHRYPRIQSIKVQFTEYKKEKGFILTSQKEDEIMKVQN▫N ▫▫▫▫▫▫▫▫▫▫SVIINCDGFYLISLKGYFSQEVNISLHYQKDEEPLFQLKKVRSVNSLMVASLTY ▫▫▫▫▫▫▫▫▫▫KDKVYLNVTTDNTSLDDFHVNGGELILIHQNPGEFCVL
c) MOXR0916 (also known as RG7888, Pogalizumab), a humanized anti-OX40 monoclonal antibody
   i. CAS Number ➔1638935-72-4
      (see www.cas.org/content/chemical-substances/faqs)
   ii. Unique Ingredient Identifier (UNII) ➔C78148TF1D
      (see www.fda./ForIndustry/DataStandards/SubstanceRegistrationSy stem-UniquelngredientldentifierUNII/default.htm)
   iii. NCI thesaurus code ➔C121376
      (see ncit.nci.nih.gov/ncitbrowser/)
d) OX40mAb24 (9B12)
   i. OX40mAb24 is a humanised version of 9B12. 9B12 is a murine IgGI, anti-OX40 mAb directed against the extracellular domain of human OX40 (CD134) (Weinberg, A.D., et al. J Immunother 29, 575-585 (2006)).
   ii. See WO2016/057667 Seq ID no.59 for OX40mAb24 VH sequence, no.29 for VL sequence (no.32 is an alternative VL):
   VH sequence QVQLQESGPGLVKPSQTLSLTCAVYGGSFSSGYWNWIRKHPGKGLEYI ▫▫▫▫▫▫▫▫▫▫▫▫▫GYISYNGITYHNPSLKSRITINRDTSKNQYSLQLNSVTPEDTAVYYCARYK ▫▫▫▫▫▫▫▫▫▫▫▫▫YDYDGGHAMDYWGQGTLVTVSS
   VL sequence DIQMTQSPSSLSASVGDRVTITCRASQDISNYLNWYQQKPGKAPKLLIYY ▫▫▫▫▫▫▫▫▫▫▫▫▫TSKLHSGVPSRFSGSGSGTDYTLTISSLQPEDFATYYCQQGSALPWTFG ▫▫▫▫▫▫▫▫▫▫▫▫▫QGTKVEIK
e) INCAGN1949
   i. See Gonzalez et al. 2016, DOI: 10.1158/1538-7445.AM2016-3204
   ii. See clinical trial NCT02923349 at clinicaltrials.gov/ct2/home
   iii. Antibody sequences are disclosed in WO2016/179517 A1:
      i. In particular, an antibody comprising the sequences: VH▫CDR1▫➔GSAMH ▫▫▫▫▫▫▫▫▫▫▫▫▫▫▫▫▫▫▫▫▫▫▫▫▫▫▫VH▫CDR2▫➔RIRSKANSYATAYAASVKG ▫▫▫▫▫▫▫▫▫▫▫▫▫▫▫▫▫▫▫▫▫▫▫▫▫▫▫VH▫CDR3▫➔GIYDSSGYDY ▫▫▫▫▫▫▫▫▫▫▫▫▫▫▫▫▫▫▫▫▫▫▫▫▫▫▫VL▫CDR1▫➔RSSQSLLHSNGYNYLD ▫▫▫▫▫▫▫▫▫▫▫▫▫▫▫▫▫▫▫▫▫▫▫▫▫▫▫VL▫CDR2▫➔LGSNRAS ▫▫▫▫▫▫▫▫▫▫▫▫▫▫▫▫▫▫▫▫▫▫▫▫▫▫▫VL▫CDR3▫➔MQALQTPLT ▫▫▫▫▫▫▫▫▫▫▫▫▫▫▫▫▫
      ii. Such as, an antibody comprising the sequences: VH▫➔ ▫▫▫▫▫▫▫▫▫▫▫▫▫▫▫▫▫▫▫▫▫▫▫▫▫▫▫EVQLVESGGGLVQPGGSLKLSCAASGFTFSGSAMHWVR ▫▫▫▫▫▫▫▫▫▫▫▫▫▫▫▫▫▫▫▫▫▫▫▫▫▫▫QASGKGLEWVGRIRSKANSYATAYAASVKGRFTISRDDS ▫▫▫▫▫▫▫▫▫▫▫▫▫▫▫▫▫▫▫▫▫▫▫▫▫▫▫KNTAYLQMNSLKTEDTAVYYCTSGIYDSSGYDYWGQGTL ▫▫▫▫▫▫▫▫▫▫▫▫▫▫▫▫▫▫▫▫▫▫▫▫▫▫▫VTVSS ▫▫▫▫▫▫▫▫▫▫▫▫▫▫▫▫▫▫▫▫▫▫▫▫▫▫▫VL▫➔ ▫▫▫▫▫▫▫▫▫▫▫▫▫▫▫▫▫▫▫▫▫▫▫▫▫▫▫DIVMTQSPLSLPVTPGEPASISCRSSQSLLHSNGYNYLDW ▫▫▫▫▫▫▫▫▫▫▫▫▫▫▫▫▫▫▫▫▫▫▫▫▫▫▫YLQKPGQSPQLLIYLGSNRASGVPDRFSGSGSGTDFTLKI ▫▫▫▫▫▫▫▫▫▫▫▫▫▫▫▫▫▫▫▫▫▫▫▫▫▫▫SRVEAEDVGVYYCMQALQTPLTFGGGTKVEIK
g) GSK3174998, a humanized IgG1 agonistic anti-OX40 monoclonal antibody (mAb)
   See clinical trial NCT02528357 at clinicaltrials.gov/ct2/home
h) PF-04518600 (PF-8600) is an investigational, fully human, monoclonal antibody (mAb) that targets OX40 protein
   See patent WO 2017/130076 A1
   See clinical trial NCT02315066 at clinicaltrials.gov/ct2/home- NCl thesaurus code ➔ C121927
      (see ncit.nci.nih.gov/ncitbrowser/)

In some embodiments, OX40 polypeptide corresponds to Genbank accession no. CAA53576, version no. CAA53576.1, record update date: Feb. 2, 2011 10:10 AM. In one embodiment, the nucleic acid encoding OX40 polypeptide corresponds to Genbank accession no. X75962, version no. X75962.1, record update date: Feb. 2, 2011 10:10 AM. In some embodiments, OX40 polypeptide corresponds to Uniprot/Swiss-Prot accession No. P43489.

### CTLA Antagonist

CTLA4 (CD152) is expressed on activated T cells and serves as a co-inhibitor to keep T cell responses in check following CD28-mediated T cell activation. CTLA4 is believed to regulate the amplitude of the early activation of naive and memory T cells following TCR engagement and to be part of a central inhibitory pathway that affects both antitumor immunity and autoimmunity. CTLA4 is expressed exclusively on T cells, and the expression of its ligands CD80 (B7.1) and CD86 (B7.2), is largely restricted to antigen-presenting cells, T cells, and other immune mediating cells. Antagonistic anti-CTLA4 antibodies that block the CTLA4 signalling pathway have been reported to enhance T cell activation. One such antibody, ipilimumab, was approved by the FDA in 2011 for the treatment of metastatic melanoma. Another anti-CTLA4 antibody, tremelimumab, was tested in phase III trials for the treatment of advanced melanoma, but did not significantly increase the overall survival of patients compared to the standard of care (temozolomide or dacarbazine) at that time.

“CTLA4 agonist” means any chemical compound or biological molecule that stimulates an immune reaction through inhibition of CTLA4 signalling.

To examine the extent of enhancement of, e.g., CTLA4 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with CTLA4 inhibitors is advantageous, because on the one hand, the ADC will directly kill the FTP positive tumor cells, while on the other hand the CTLA4 inhibitor will engage the patient’s own immune system to eliminate the cancer cells. Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor. The resulting release of tumor associated antigens from cells killed with the PBD dimer will trigger the immune system, which will be further enhanced by the use of CTLA4 inhibitors expressed on a large proportion of tumour infiltrating lymphocytes (TILs) from many different tumour types.

The major function of CTLA4 (CD152) is to regulate the amplitude of the early stages of T cell activation, and as such it counteracts the activity of the T cell co-stimulatory receptor, CD28, In the tumor microenvironment. Blockade of the CTLA4 pathway may therefore enhance enhancement of effector CD4+T cell activity, while it inhibits TReg cell-dependent immunosuppression. Therefore it will be beneficial to target a FTP(+) tumor with the ADC, causing the antigenic cell death, while the CTLA4 blockade induces a stronger immune, durable response.

Specific CTLA4 antagonists suitable for use as secondary agents in the present disclosure include:
ipilimumab
   i. CAS Number ➔ 477202-00-9
      (see www.cas.org/content/chemical-substances/faqs)
   ii. Unique Ingredient Identifier (UNII) ➔ 6T8C155666
      (see www.fda./ForIndustry/DataStandards/SubstanceRegistrationSy stem-UniqueIngredientIdentifierUNII/default.htm)
b) Tremelimumab
   i. CAS Number ➔ 745013-59-6
      (see www.cas.org/content/chemical-substances/faqs)
   ii. Unique Ingredient Identifier (UNII) ➔ QEN1X95CIX
      (see www.fda./ForIndustry/DataStandards/SubstanceRegistrationSy stem-UniqueIngredientIdentifierUNII/default.htm)
   iii. VH sequence GVVQPGRSLRLSCAASGFTFSSYGMHWVRQAPGKGLEWVAVIWYDGS ▫▫▫▫▫▫▫▫▫▫▫▫▫NKYYADSVKGRFTISRDNSKNTLYLQMNSLRAEDTAVYYCARDPRGATL ▫▫▫▫▫▫▫▫▫▫▫▫▫YYYYYGMDVWGQGTTVTVSSASTKGPSVFPLAPCSRSTSESTAALG ▫▫▫▫▫▫▫▫▫▫▫▫▫CLVKDYFPEPVTVSWNSGALTSGVH▫[SEQ▫ID▫NO.▫1]
   iv. VL sequence PSSLSASVGDRVTITCRASQSI▫NSYLDWYQQKPGKAPKLLIYAASSLQSG ▫▫▫▫▫▫▫▫▫▫▫▫▫VPSRFSGSGSGTDFTLTISSLQPEDFATYYCQQYYSTPFTFGPGTKVEIK ▫▫▫▫▫▫▫▫▫▫▫▫▫RTVAAPSVFIFPPSDEQLKSGTASVVCLLNNFYPREAKV▫[SEQ▫ID▫NO.▫2]

In some embodiments, CTLA polypeptide corresponds to Genbank accession no. AAL07473, version no. AAL07473.1, record update date: Mar. 11, 2010 01:28 AM. In one embodiment, the nucleic acid encoding CTLA4 polypeptide corresponds to Genbank accession no. AF414120, version no. AF414120.1, record update date: Mar. 11, 2010 01:28 AM . In some embodiments, OX40 polypeptide corresponds to Uniprot/Swiss-Prot accession No. P16410.

### Fludarabine and Cytarabine

Combination of agents with different action mechanisms is an established therapeutic principle for combating cancer. It can be a way of increasing anti-tumour activity when a synergic effect is shown and/or when reduced toxicity is observed. Antibody-drug conjugates, including those with a PBD warhead, may be particularly suited as combination partners because they are more targeted compared to conventional chemotherapy. As PBD dimers cross-link DNA in a covalent fashion, combining them with other agents that interfere with DNA synthesis via a different mechanism is likely to provide a benefit. Examples of such potential combinations are Fludarabine and Cytarabine.

### Fludarabine

Fludarabine or fludarabine phosphate (Fludara) is a chemotherapy drug used in the treatment of hematological malignancies such as leukemias and lymphomas. It is a purine analog, which interferes with DNA by interfering with ribonucleotide reductase (RNAR) and DNA polymerase. It is active against both dividing and resting cells. Fludarabine has also been shown to suppress ERCC1 transcription and this may explain the observed synergy between Fludarabine and the PBD Dimer SJG136 (SG2000) against chronic lymphocytic leukaemia cells. CLAG/CLAG-M—Cladribine is another purine analogue that inhibits RNR.

Combining the ADC, which targets First Target Protein (FTP) positive lymphomas and leukemias, with Fludarabine is advantageous, because on the one hand, the ADC will directly kill the FTP positive tumor cells via a mechanisms depending on DNA cross-linking resulting in apoptosis, while on the other hand the Fludarabine will inhibit the cells RNA and DNA polymerase, while also suppressing the DNA repair enzymes needed to resolve the DNA cross-links induced by the PBD dimer.

To show that the ADC works synergistically with Fludarabine, a panel of FTP(+) cell lines will be co-treated with a range of concentration of both the ADC and Fludarabine. As negative controls, the same panel of cell lines will be co-treated with a range of concentrations of Fludarabine and a non-targeted control ADC or with a range of concentration of the ADC and vehicle. After incubation, two parameters will be measured: the amount of surface FTP (as determined by flow cytometry) and the in vitro cytotoxicity of the combinations (as determined by CellTiter-Glo® or MTS assays). Cytotoxic synergy is calculated by transforming the cell viability data into fraction affected, and calculating the combination index using the CalcuSyn analysis program.
CAS Number ➔ 21679-14-1
   (see www.cas.org/content/chemical-substances/faqs)
ii. NCBI Pubchem reference ➔ 657237
   (see pubchem.ncbi.nlm.nih.gov/)
iii. IUPHAR/BPS reference ➔ 4802
   (see www.guidetopharmacology.org/)
iv. Unique Ingredient Identifier (UNII) ➔ 1X9VK9O1SC
   (see www.guidetopharmacology.org/)(see www.fda.gov/Forlndustry/DataStandards/SubstanceRegistrationSystem-UniquelngredientldentifierUNII/default.htm) 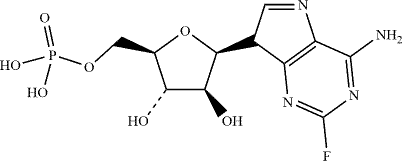 [SMILES: Nc1nc(F)nc2c1N=CC2[C@@H]1O[C@H](COP(=O)(O)O)[C@@H](O)[C@@H]1O]
   Formula VII, Fludarabine: [(2R,3R,4S,5R)-5-(6-amino-2-fluoro-purin-9-yl)- 3,4-dihydroxy-oxolan-2-yl]methoxyphosphonic acid

### Cytarabine

Cytarabine or cytosine arabinoside (Cytosar-U or Depocyt) is a antimetabolic chemotherapy drug used in the treatment of hematological malignancies such as acute myeloid leukemia (AML) and non-Hodgkin lymphoma. It is also known as ara-C (arabinofuranosyl cytidine). It kills cancer cells by interfering with DNA synthesis. It is actively metabolized to cytosine arabinoside triphosphate, which damages DNA when the cell cycle holds in the S phase (synthesis of DNA). Rapidly dividing cells, which require DNA replication for mitosis, are therefore most affected. Cytosine arabinoside also inhibits both DNA and RNA polymerases and nucleotide reductase enzymes needed for DNA synthesis.

Combining the ADC, which targets First Target Protein (FTP) positive lymphomas and leukemias, with Cytarabine is advantageous, because on the one hand, the ADC will directly kill the FTP positive tumor cells via a mechanisms depending on DNA cross-linking resulting in apoptosis, while on the other hand the Cytarabine will inhibit the cells RNA and DNA polymerase, while also suppressing DNA synthesis.

To show that the ADC works synergistically with Cytarabine, a panel of FTP(+) cell lines will be co-treated with a range of concentration of both the ADC and Cytarabine. As negative controls, the same panel of cell lines will be co-treated with a range of concentrations of Cytarabine and a non-targeted control ADC or with a range of concentration of the ADC and vehicle. After incubation, two parameters will be measured: the amount of surface FTP (as determined by flow cytometry) and the in vitro cytotoxicity of the combinations (as determined by CellTiter-Glo® or MTS assays). Cytotoxic synergy is calculated by transforming the cell viability data into fraction affected, and calculating the combination index using the CalcuSyn analysis program).

CAS Number ➔ 147-94-4
   (see www.cas.org/content/chemical-substances/faqs)
ii. NCBI Pubchem reference ➔ 6253
   (see pubchem.ncbi.nlm.nih.gov/)
iii. IUPHAR/BPS reference ➔ 4827
   (see www.guidetopharmacology.org/)
iv. Unique Ingredient Identifier (UNII) ➔ 04079A1 RDZ
   (see www.fda.gov/Forlndustry/DataStandards/SubstanceRegistrationSystem-UniquelngredientldentifierUNII/default.htm) 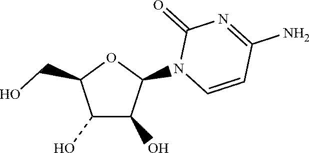 [SMILES: Nc1ccn([C@@H]2O[C@H](CO)[C@@H](O)[C@@H]2O)c(=O)n1]
   Formula VIII, Cytarabine: 4-amino-1-[(2R,3S,4R,5R)-3,4-dihydroxy-5-(hydroxymethyl)oxolan-2-yl] pyrimidin-2-one

### Hypomethylating Agent

The term “hypomethylating agent” refers to a class of compounds that interfere with DNA methylation which is the addition of a methyl group to the 5- position of the cytosine pyrimidine ring or the nitrogen in position 6 of the adenine purine ring. DNA methylation stably alters the gene expression pattern in cells i.e. decrease gene expression (i.e. for the Vitamin D receptor). Hypomethylating agent are compounds that can inhibit methylation, resulting in the expression of the previously hypermethylated silenced genes. Cytidine analogs such as 5-azacytidine (azacitidine) and 5-aza-2′-deoxycytidine (decitabine are the most commonly used Hypomethylating agent. These compounds work by binding to the enzymes that catalyse the methylation reaction, i.e. DNA methyltransferases.

To examine the extent of hypomethylation, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with a hypomethylating agent is advantageous, because on the one hand the ADC will directly kill the FTP positive tumor cells, while on the other hand the a hypomethylating agent will interfere with DNA methylation. This interference is by way of causing demethylation in that sequence, which adversely affects the way that cell regulatory proteins are able to bind to the DNA/RNA substrate. This activity synergises with the ADC because PBD dimers cross-link DNA in a covalent fashion, so combining them with other agents that interfere with DNA synthesis via a different mechanism provides a benefit.

Specific Hypomethylating agents suitable for use as secondary agents in the present disclosure include:
a) 5-azacytidine (azacitidine)
   i. CAS Number ➔ 320-67-2
      (see www.cas.org/content/chemical-substances/faqs)
   ii. NCBI Pubchem reference ➔ 9444 (
      see pubchem.ncbi.nlm.nih.gov/)
   iii. IUPHAR/BPS reference ➔ 6796
      (see www.guidetopharmacology.org/)
   iv. Unique Ingredient Identifier (UNII) ➔ M801 H13NRU
      (see www.fda./ForIndustry/DataStandards/SubstanceRegistrationSy stem-UniqueIngredientIdentifierUNII/default.htm) 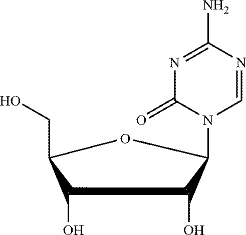
      Formula IX, 5-azacytidine: 4-Amino-1-β-D-ribofuranosyl-1,3,5-triazin-2(1H)-one
b) 5-aza-2′-deoxycytidine (decitabine)
   i. CAS Number ➔ 2353-33-5
      (see www.cas.org/content/chemical-substances/faqs)
   ii. NCBI Pubchem reference ➔ 451668
      (see pubchem.ncbi.nlm.nih.gov/)
   iii. IUPHAR/BPS reference ➔ 6805
      (see www.guidetopharmacology.org/)
   iv. Unique Ingredient Identifier (UNII) ➔ 776B62CQ27
      (see www.fda./ForIndustry/DataStandards/SubstanceRegistrationSy stem-UniqueIngredientIdentifierUNII/default.htm) 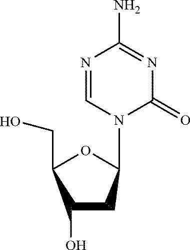 [SMILES: Nc1ncn([C@H]2C[C@@H](O)[C@@H](CO)O2)c(=O)n1]
      Formula X, b) 5-aza-2′-deoxycytidine: 4-Amino-1-(2-deoxy-β-D-erythro-pentofuranosyl)-1,3,5-triazin-2(1H)-one

### Agents that Upregulate HER2 Expression

An agent that “upregulates HER2 expression” means any chemical compound or biological molecule that increase the amount of HER2 protein on a tumour cell surface.

To examine the extent of enhancement samples or assays comprising a given, e.g., protein, gene, cell, or organism, are treated with a potential activating agent and are compared to control samples treated with an inactive control molecule. Control samples are assigned a relative expression value of 100%. Activation is achieved when the expression value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Specific agents that upregulate HER2 expression suitable for use as secondary agents in the present disclosure include:
a) gemcitabine
   i. CAS Number ➔95058-81-4
      (see www.cas.org/content/chemical-substances/faqs)
   ii. NCBI Pubchem reference ➔ 60750
      (see pubchem.ncbi.nlm.nih.gov/)
   iii. DrugBank reference ➔DB00441
      (see www.drugbank.ca/)
      iv. Unique Ingredient Identifier (UNII) ➔B76N6SBZ8R
      (see www.fda./ForIndustry/DataStandards/SubstanceRegistrationSy stem-UniqueIngredientIdentifierUNII/default.htm) 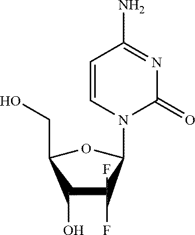
      Formula XI, Gemcitabine: 4-Amino-1-(2-deoxy-2,2-difluoro-β-D-erythro-pentofuranosyl)pyrimidin-2(1H)-on
b) tamoxifen
   i. CAS Number ➔10540-29-1
      (see www.cas.org/content/chemical-substances/faqs)
   ii. NCBI Pubchem reference ➔2733526
      (see pubchem.ncbi.nlm.nih.gov/)
   iii. DrugBank reference ➔DB00675
      (see www.drugbank.ca/)
   iv. Unique Ingredient Identifier (UNII) ➔094ZI81Y45
      (see www.fda./ForIndustry/DataStandards/SubstanceRegistrationSy stem-UniqueIngredientIdentifierUNII/default.htm) 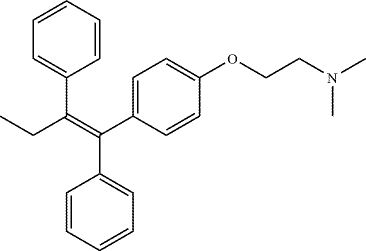 [SMILES: CC/C(=C(\c1ccccc1)c1ccc(OCCN(C)C)cc1)c1ccccc1]
      Formula XII, Tamoxifen: (Z)-2-[4-(1,2-diphenylbut-1-enyl)phenoxy]-N,N-dimethylethanamine

### Anti-CD20 Agents

In some embodiments the anti-CD20 agent is administered in combination with the ADC as a secondary agent (i.e. anti-CD20 agent = secondary agent). That is, it is envisaged that a combination of [ADC + anti-CD20 agent] is administered to the individual in combination for example, [ADCx19 + Rituximab] or [ADCx22 + Rituximab].

In some embodiments, the anti-CD20 agent is administered in combination with the ADC as a tertiary agent (i.e. anti-CD20 agent = tertiary agent), in further combination with a secondary agent as described herein (such as a Bruton’s Tyrosine Kinase inhibitor (BTKi), a PD1 antagonist, a PD-L1 antagonist, a GITR agonist, an OX40 agonist, a CTLA-4 antagonist, Fludarabine or Cytarabine, a hypomethylating agent, or an agent that upregulates HER2 expression). That is, it is envisaged that a combination of [ADC + secondary agent + anti-CD20 agent] is administered to the individual in combination; for example, [ADCx19 + secondary agent + Rituximab] or [ADCx22 + secondary agent + Rituximab].

In some embodiments the individual is administered a combination of [ADC + Cytarabine + anti-CD20 agent], such as [ADCx19 + Cytarabine + Rituximab] or [ADCx22 + Cytarabine + Rituximab].

In some embodiments the individual is administered a combination of [ADC + Fludarabine + anti-CD20 agent], such as [ADCx19 + Fludarabine + Rituximab] or [ADCx22 + Fludarabine + Rituximab].

Preferably, in embodiments where the administered combination comprises an anti-CD20 agent, the ADC is an anti-CD19 ADC such as ADCx19.

The anti-CD20 agent may be an anti-CD20 antibody or antibody-conjugate. Suitable anti-CD20 antibodies or antibody-conjugates include rituximab, obinutuzumab, Ibritumomab tiuxetan, tositumomab, Ofatumumab, Ocaratuzumab, Ocrelizumab, and Veltuzumab. Preferably the anti-CD20 agent is rituximab.

CD20 is a 33-37 kDa, non-glycosylated phosphoprotein expressed on the surface of the majority of B-cells, both normal and malignant. The biology of CD20 is still relatively poorly understood - it has no known natural ligand and CD20 knockout mice display an almost normal phenotype, with only a slightly decreased T-independent immune response reported. CD20 is resident in lipid raft domains of the plasma membrane where it has been suggested to function as a store-operated calcium channel following ligation of the B-cell receptor for antigen (see Boross et al., Am J Cancer Res. 2012; 2(6): 676-690).

“Anti-CD20 agent” is used herein to mean any agent that specifically binds to and/or inhibits a biological activity of CD20. A preferred class of anti-CD20 agents is antibodies or antibody-conjugates that specifically bind CD20.

As used herein, “specifically binds CD20” is used to mean the antibody binds CD20 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD20 with an association constant (Kₐ) at least 2, 3, 4, 5, 10, 20, 50, 100, 200, 500, 1000, 2000, 5000, 10⁴, 10⁵ or 10⁶-fold higher than the antibody’s association constant for BSA, when measured at physiological conditions. The antibodies may bind CD20 with a high affinity. For example, in some embodiments the antibody can bind CD20 with a K_{D} equal to or less than about 10-⁶ M, such as 1 x 10⁻⁶, 10⁻⁷, 10⁻⁸, 10⁻⁹,10⁻¹⁰, 10⁻¹¹, 10⁻¹², 10⁻¹³ or 10⁻¹⁴.

In some embodiments, CD20 polypeptide corresponds to Genbank accession no. CAA31046, version no. CAA31046.1, record update date: Feb. 2, 2011 10:09 AM . In one embodiment, the nucleic acid encoding CD20 polypeptide corresponds to Genbank accession no X12530, version no. X12530.1, record update date: Feb. 2, 2011 10:09 AM. In some embodiments, CD20 polypeptide corresponds to Uniprot/Swiss-Prot accession No. P11836.

To show that anti-CD19 ADCs and secondary agent combination works synergistically with the anti-CD20 agent, a panel of CD19 (+) cell lines will be co-treated with a range of concentrations of both anti-CD19 ADC / secondary agent and the anti-CD20 agent. As negative controls, the same panel of cell lines will be treated with a range of concentrations of the anti-CD20 agent or with a range of concentration of anti-CD19 ADC / secondary agent and vehicle. After incubation, two parameters will be measured: the amount of surface CD19 (as determined by flow cytometry) and the in vitro cytotoxicity of the combinations (as determined by MTS assays). To determine the cytotoxicity, Cell viability is measured by adding MTS per well and incubating for 4 hours at 37° C. Percentage cell viability is calculated compared to the untreated control. Cytotoxic synergy is calculated by transforming the cell viability data into fraction affected, and calculating the combination index using the CalcuSyn analysis program.

Anti-CD20 agents suitable for use in the present disclosure include:
a) Rituximab
   i. CAS Number ➔174722-31-7
      (see www.cas.org/content/chemical-substances/faqs)
   ii. Drugbank reference ➔DB00073
      (see www.drugbank.ca/)
   iii. Unique Ingredient Identifier (UNII) ➔4F4X42SYQ6
      (see www.fda.gov/Forlndustry/DataStandards/SubstanceRegistrationSystem-UniqueIngredientIdentifierUNII/default.htm)
   iv. Heavy chain sequence: QVQLQQPGAELVKPGASVKMSCKASGYTFTSYNMHWVKQTPGRGLEW ▫▫▫▫▫▫▫▫▫▫▫▫▫IGAIYPGNGDTSYNQKFKGKATLTADKSSSTAYMQLSSLTSEDSAVYYCA ▫▫▫▫▫▫▫▫▫▫▫▫▫RSTYYGGDWYFNVWGAGTTVTVSAASTKGPSVFPLAPSSKSTSGGTAA ▫▫▫▫▫▫▫▫▫▫▫▫▫LGCLVKDYFPEPVTVSWNSGALTSGVHTFPAVLQSSGLYSLSSVVTVPS ▫▫▫▫▫▫▫▫▫▫▫▫▫SSLGTQTYICNVNHKPSNTKVDKKAEPKSCDKTHTCPPCPAPELLGGPS ▫▫▫▫▫▫▫▫▫▫▫▫▫VFLFPPKPKDTLMISRTPEVTCVVVDVSHEDPEVKFNWYVDGVEVHNAK ▫▫▫▫▫▫▫▫▫▫▫▫▫TKPREEQYNSTYRVVSVLTVLHQDWLNGKEYKCKVSNKALPAPIEKTISK ▫▫▫▫▫▫▫▫▫▫▫▫▫AKGQPREPQVYTLPPSRDELTKNQVSLTCLVKGFYPSDIAVEWESNGQP ▫▫▫▫▫▫▫▫▫▫▫▫▫ENNYKTTPPVLDSDGSFFLYSKLTVDKSRWQQGNVFSCSVMHEALHNH ▫▫▫▫▫▫▫▫▫▫▫▫▫YTQKSLSLSPGK
   Light chain sequence: QIVLSQSPAI▫LSASPGEKVTMTCRASSSVSYI▫HWFQQKPGSSPKPWIYAT ▫▫▫▫▫▫▫▫▫▫▫▫▫SNLASGVPVRFSGSGSGTSYSLTISRVEAEDAATYYCQQWTSNPPTFGG ▫▫▫▫▫▫▫▫▫▫▫▫▫GTKLEIKRTVAAPSVFIFPPSDEQLKSGTASVVCLLNNFYPREAKVQWKV ▫▫▫▫▫▫▫▫▫▫▫▫▫DNALQSGNSQESVTEQDSKDSTYSLSSTLTLSKADYEKHKVYACEVTHQ ▫▫▫▫▫▫▫▫▫▫▫▫▫GLSSPVTKSFNRGEC
b) obinutuzumab
   i. CAS Number ➔ 949142-50-1
      (see www.cas.org/content/chemical-substances/faqs)
   ii. Unique Ingredient Identifier (UNII) ➔ 043472U9X8
      (see www.fda.gov/Forlndustry/DataStandards/SubstanceRegistrationSystem-UniqueIngredientIdentifierUNII/default.htm)
c) Ibritumomab tiuxetan
   i. CAS Number ➔ 206181-63-7
      (see www.cas.org/content/chemical-substances/faqs)
   ii. Drugbank reference ➔ DB00078
      (see www.drugbank.ca/)
   iii. Unique Ingredient Identifier (UNII) ➔ 4Q52C550XK
      (see www.fda.gov/Forlndustry/DataStandards/SubstanceRegistrationSystem-UniqueIngredientIdentifierUNII/default.htm)
d) Tositumomab
   i. CAS Number ➔ 208921-02-2
      (see www.cas.org/content/chemical-substances/faqs)
   ii. Drugbank reference ➔ DB00081
      (see www.drugbank.ca/)
   iii. Unique Ingredient Identifier (UNII) ➔ 0343IGH41U
      (see www.fda.gov/Forlndustry/DataStandards/SubstanceRegistrationSystem-UniqueIngredientIdentifierUNII/default.htm)
e) Ofatumumab
   i. CAS Number ➔ 679818-59-8
      (see www.cas.org/content/chemical-substances/faqs)
   ii. Drugbank reference ➔ DB06650
      (see www.drugbank.ca/)
   iii. Unique Ingredient Identifier (UNII) ➔ M95KG522R0
      (see www.fda.gov/Forlndustry/DataStandards/SubstanceRegistrationSystem-UniqueIngredientIdentifierUNII/default.htm)
f) Ocaratuzumab
   i. CAS Number ➔ 1169956-08-4
      (see www.cas.org/content/chemical-substances/faqs)
g) Ocrelizumab
   i. CAS Number ➔ 637334-45-3
      (see www.cas.org/content/chemical-substances/faqs)
   ii. Unique Ingredient Identifier (UNII) ➔ A10SJL62JY
      (see www.fda.gov/Forlndustry/DataStandards/SubstanceRegistrationSystem-UniqueIngredientIdentifierUNII/default.htm)
h) Veltuzumab
   i. CAS Number ➔ 728917-18-8
      (see www.cas.org/content/chemical-substances/faqs)
   ii. Unique Ingredient Identifier (UNII) ➔ BPD4DGQ314
      (see www.fda.gov/Forlndustry/DataStandards/SubstanceRegistrationSystem-UniqueIngredientIdentifierUNII/default.htm)

### Advantageous Properties of the Described Combinations

Both the ADC and secondary agent when used as a single agent in isolation have demonstrated clinical utility - for example, in the treatment of cancer. However, as described herein, combination of the ADC and secondary agent is expected to provide one or more of the following advantages over treatment with either ADC or secondary agent alone:
1) effective treatment of a broader range of cancers;
2) effective treatment of resistant or refractory forms of disorders such as cancer, and individuals with disorders such as cancer who have relapsed after a period of remission;
3) increased response rate to treatment; and / or
4) Increased durability of treatment.

Effective treatment of a broader range of cancers as used herein means that following treatment with the combination a complete response is observed with a greater range of recognised cancer types. That is, a complete response is seen from cancer types not previously reported to completely respond to ADC, secondary agent, or anti-CD20 agent alone (or in combinations of two of the three elements).

For example, the combination of the anti-CD19 ADC, ADCx19, and the anti-CD20 agent, Rituximab, have been demonstrated to show synergistically enhanced cytotoxicity (see Example 4 and FIG. 2 herein).

The combination of the anti-CD19 ADC, ADCx19, and Cytarabine have also been demonstrated to show synergistically enhanced cytotoxicity (see Example 5 and FIG. 3), as has the combination of the anti-CD22 ADC, ADCx22, and Cytarabine (see Example 6 and FIG. 4A). The combination of ADCx22 and Fludarabine also shows synergistically enhanced cytotoxicity (see Example 6 and FIG. 4B).

Consistent with the in vitro data described above, in vivo data from a WSU-DLCL2 xenograft study indicated synergistically enhanced anti-tumour activity for the ADCx19 / Cytarabine combination and the ADCx19 / Rituximab combination (see Example 7 and FIG. 5).

Effective treatment of a resistant, refractory, or relapsed forms as used herein means that following treatment with the combination a complete response is observed in individuals that are either partially or completely resistant or refractory to treatment with ADC, secondary agent, or anti-CD20 agent alone (or in combinations of two of the three elements; for example, individuals who show no response or only partial response following treatment with any agent alone (or combinations of 2 of the 3 elements), or those with relapsed disorder). In some embodiments, a complete response following treatment with the ADC / secondary agent / anti-CD20 agent combination is observed at least 10% of individuals that are either partially or completely resistant or refractory to treatment with ADC, secondary agent, or anti-CD20 agent alone (or in combinations of two of the three elements. In some embodiments, a complete response following treatment with the ADC / secondary agent / anti-CD20 agent combination is observed at least 20%, at least 30%, at least 40%, at least 50%, at least 60%, at least 70%, at least 80%, at least 90%, at least 95%, at least 98%, or at least 99% of individuals that are either partially or completely resistant or refractory to treatment with ADC, secondary agent, or anti-CD20 agent alone (or in combinations of two of the three elements.

Increased response rate to treatment as used herein means that following treatment with the combination a complete response is observed in a greater proportion of individuals than is observed following treatment with ADC, secondary agent, or anti-CD20 agent alone (or in combinations of two of the three elements. In some embodiments, a complete response following treatment with the ADC / secondary agent / anti-CD20 agent combination is observed at least 10% of treated individuals. In some embodiments, a complete response following treatment with the ADC / secondary agent / anti-CD20 agent combination is observed at least 20%, at least 30%, at least 40%, at least 50%, at least 60%, at least 70%, at least 80%, at least 90%, at least 95%, at least 98%, or at least 99% of treated individuals.

Increased durability of treatment as used herein means that average duration of complete response in individuals treated with the triple combination is longer than in individuals who achieve complete response following treatment with ADC, secondary agent, or anti-CD20 agent alone (or in combinations of two of the three elements). In some embodiments, the average duration of a complete response following treatment with the ADC / secondary agent / anti-CD20 agent combination is at least 6 months. In some embodiments, the average duration of a complete response following treatment with the ADC / secondary agent / anti-CD20 agent combination is at least 12 months, at least 18 months, at least 24 months, at least 3 years, at least 4 years, at least 5 years, at least 6 years, at least 7 years, at least 8 years, at least 9 years, at least 10 years, at least 15 years, or at least 20 years.

‘Complete response’ is used herein to mean the absence of any clinical evidence of disease in an individual. Evidence may be assessed using the appropriate methodology in the art, for example CT or PET scanning, or biopsy where appropriate. The number of doses required to achieve complete response may be one, two, three, four, five, ten or more. In some embodiments the individuals achieve complete response no more than a year after administration of the first dose, such as no more than 6 months, no more than 3 months, no more than a month, no more than a fortnight, or no more than a week after administration of the first dose.

### Treated Disorders

The combined therapies described herein include those with utility for anticancer activity. In particular, in certain aspects the therapies include an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

Thus, in one aspect, the present disclosure provides combined therapies comprising administering an ADC which binds a first target protein for use in therapy, wherein the method comprises selecting a subject based on expression of the target protein.

In one aspect, the present disclosure provides a combined therapy with a label that specifies that the therapy is suitable for use with a subject determined to be suitable for such use. The label may specify that the therapy is suitable for use in a subject has expression of the first target protein, such as overexpression of the first target protein. The label may specify that the subject has a particular type of cancer.

The first target protein is preferably CD19 or CD22. The cancer may be lymphoma, such as non-Hodgkins lymphoma. The label may specify that the subject has a CD19+ or CD22+ lymphoma.

In a further aspect there is also provided a combined therapy as described herein for use in the treatment of a proliferative disease. Another aspect of the present disclosure provides the use of a conjugate compound in the manufacture of a medicament for treating a proliferative disease.

One of ordinary skill in the art is readily able to determine whether or not a candidate combined therapy treats a proliferative condition for any particular cell type. For example, assays which may conveniently be used to assess the activity offered by a particular compound are described below.

The combined therapies described herein may be used to treat a proliferative disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi’s sarcoma, melanoma), lymphomas, leukemias, psoriasis, bone diseases, fibroproliferative disorders (e.g. of connective tissues), and atherosclerosis. Cancers of interest include, but are not limited to, leukemias and ovarian cancers.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Proliferative disorders of particular interest include, but are not limited to, non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL). [Fielding A., Haematologica. 2010 Jan; 95(1): 8-12].

The proliferative disease may be characterised by the presence of a neoplasm comprising both CD19+ve and CD19-ve cells. The proliferative disease may be characterised by the presence of a neoplasm comprising both CD22+ve and C22-ve cells.

The proliferative disease may be characterised by the presence of a neoplasm composed of CD19-ve neoplastic cells, optionally wherein the CD19-ve neoplastic cells are associated with CD19+ve neoplastic or non-neoplastic cells. The proliferative disease may be characterised by the presence of a neoplasm composed of CD22-ve neoplastic cells, optionally wherein the CD22-ve neoplastic cells are associated with CD22+ve neoplastic or non-neoplastic cells.

The target cancer or cancer cells may be all or part of a solid tumour.

“Solid tumor” herein will be understood to include solid haematological cancers such as lymphomas (Hodgkin’s lymphoma or non-Hodgkin’s lymphoma) which are discussed in more detail herein.

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 Jan;27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

It is contemplated that the combined therapies of the present disclosure may be used to treat various diseases or disorders, e.g. characterized by the overexpression of a tumor antigen. Exemplary conditions or hyperproliferative disorders include benign or malignant tumors; leukemia, haematological, and lymphoid malignancies. Others include neuronal, glial, astrocytal, hypothalamic, glandular, macrophagal, epithelial, stromal, blastocoelic, inflammatory, angiogenic and immunologic, including autoimmune disorders and graft-versus-host disease (GVHD).

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Autoimmune diseases for which the combined therapies may be used in treatment include rheumatologic disorders (such as, for example, rheumatoid arthritis, Sjogren’s syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g. ulcerative colitis and Crohn’s disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener’s granulomatosis, and polyarteriitis), autoimmune neurological disorders (such as, for example, multiple sclerosis, opsoclonus myoclonus syndrome, myasthenia gravis, neuromyelitis optica, Parkinson’s disease, Alzheimer’s disease, and autoimmune polyneuropathies), renal disorders (such as, for example, glomerulonephritis, Goodpasture’s syndrome, and Berger’s disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet’s disease, Raynaud’s syndrome, organ transplant, graft-versus-host disease (GVHD), and autoimmune endocrine disorders (such as, for example, diabetic-related autoimmune diseases such as insulin-dependent diabetes mellitus (IDDM), Addison’s disease, and autoimmune thyroid disease (e.g. Graves’ disease and thyroiditis)). More preferred such diseases include, for example, rheumatoid arthritis, ulcerative colitis, ANCA-associated vasculitis, lupus, multiple sclerosis, Sjogren’s syndrome, Graves’ disease, IDDM, pernicious anemia, thyroiditis, and glomerulonephritis.

In some aspects, the subject has a proliferative disorder selected from non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL). [Fielding A., Haematologica. 2010 Jan; 95(1): 8-12].

In certain aspects, the subject has diffuse large B cell lymphoma.

In some aspects, the subject has a proliferative disease characterised by the presence of a neoplasm comprising both CD19+ve and CD19-ve cells. In some aspects, the subject has a proliferative disease characterised by the presence of a neoplasm comprising both CD22+ve and C22-ve cells.

The proliferative disease may be characterised by the presence of a neoplasm composed of CD19-ve neoplastic cells, optionally wherein the CD19-ve neoplastic cells are associated with CD19+ve neoplastic or non-neoplastic cells. The proliferative disease may be characterised by the presence of a neoplasm composed of CD22-ve neoplastic cells, optionally wherein the CD22-ve neoplastic cells are associated with CD22+ve neoplastic or non-neoplastic cells.

The target neoplasm or neoplastic cells may be all or part of a solid tumour In some aspects, the subject has a solid tumour.

“Solid tumor” herein will be understood to include solid haematological cancers such as lymphomas (Hodgkin’s lymphoma or non-Hodgkin’s lymphoma) which are discussed in more detail herein.

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 Jan;27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

### Patient Selection

In certain aspects, the individuals are selected as suitable for treatment with the combined treatments before the treatments are administered.

As used herein, individuals who are considered suitable for treatment are those individuals who are expected to benefit from, or respond to, the treatment. Individuals may have, or be suspected of having, or be at risk of having cancer. Individuals may have received a diagnosis of cancer. In particular, individuals may have, or be suspected of having, or be at risk of having, lymphoma. In some cases, individuals may have, or be suspected of having, or be at risk of having, a solid cancer that has tumour associated non-tumor cells that express a first target protein, such as infiltrating cells that express a first target protein.

In some aspects, individuals are selected on the basis of the amount or pattern of expression of a first target protein. In some aspects, the selection is based on expression of a first target protein at the cell surface.

In some aspects, individuals are selected on the basis they have a neoplasm comprising both CD19+ve and CD19-ve cells. The neoplasm may be composed of CD19-ve neoplastic cells, optionally wherein the CD19-ve neoplastic cells are associated with CD19+ve neoplastic or non-neoplastic cells. The neoplasm or neoplastic cells may be all or part of a solid tumour. The solid tumour may be partially or wholly CD19-ve. In some aspects, individuals are selected on the basis they have a neoplasm comprising both CD22+ve and CD22-ve cells. The neoplasm may be composed of CD22-ve neoplastic cells, optionally wherein the CD22-ve neoplastic cells are associated with CD22+ve neoplastic or non-neoplastic cells. The neoplasm or neoplastic cells may be all or part of a solid tumour. The solid tumour may be partially or wholly CD22-ve.

In certain aspects, the target is a second target protein. In some aspects, the selection is based on expression of a second target protein at the cell surface.

In some aspects, the selection is based on levels of both a first target protein and a second target protein at the cell surface.

In some cases, expression of the target in a particular tissue of interest is determined. For example, in a sample of lymphoid tissue or tumor tissue. In some cases, systemic expression of the target is determined. For example, in a sample of circulating fluid such as blood, plasma, serum or lymph.

In some aspects, the individual is selected as suitable for treatment due to the presence of target expression in a sample. In those cases, individuals without target expression may be considered not suitable for treatment.

In other aspects, the level of target expression is used to select a individual as suitable for treatment. Where the level of expression of the target is above a threshold level, the individual is determined to be suitable for treatment.

In some aspects, the presence of a first target protein and/or a second target protein in cells in the sample indicates that the individual is suitable for treatment with a combination comprising an ADC and a secondary agent. In other aspects, the amount of first target protein and/or a second target protein expression must be above a threshold level to indicate that the individual is suitable for treatment. In some aspects, the observation that first target protein and/or a second target protein localisation is altered in the sample as compared to a control indicates that the individual is suitable for treatment.

In some aspects, an individual is indicated as suitable for treatment if cells obtained from lymph node or extra nodal sites react with antibodies against first target protein and/or a second target protein as determined by IHC.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a first target protein. In some aspects disclosed herein, a patient is determined to be suitable for treatment if at least at least 10% of the cells in the sample express a first target protein.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a second target protein. In some aspects disclosed herein, a patient is determined to be suitable for treatment if at least at least 10% of the cells in the sample express a second target protein.

In some aspects, the individual is selected as suitable for treatment based on their current or previous treatment regime. In some embodiments the individual is selected for treatment with the ADC and/or secondary agent combination if the individual has been treated with an anti-CD20 agent. In some embodiments the individual is selected for treatment with the ADC and/or secondary agent combination if the individual is being treated with an anti-CD20 agent. In some cases the individual is selected for treatment if they are refractory to treatment (or further treatment) with the anti-CD20 agent. In some cases the anti-CD20 agent may be Rituximab. In embodiments where the individual is undergoing, or has undergone, treatment with an anti-CD20 agent, the ADC and/or secondary agent combination may be administered in combination with an anti-CD20 agent, or without continued administration of the anti-CD20 agent. The ADC may be an anti-CD19 ADC, such as ADCx19. The ADC may be an anti-CD22 ADC, such as ADCx22.

In some embodiments the ADC and/or secondary agent combination is administered to the selected individual in combination with an anti-CD20 agent. In some embodiments the ADC and/or secondary agent combination is administered to the selected individual without continued administration of an anti-CD20 agent. The anti-CD20 agent is preferably Rituximab. The ADC may be an anti-CD19 ADC, such as ADCx19. The ADC may be an anti-CD22 ADC, such as ADCx22.

The term ‘refractory to treatment (or further treatment) with the anti-CD20 agent’ is used herein to mean that the disorder (such as cancer) does not respond, or has ceased to respond, to administration of the anti-CD20 agent when administered as a monotherapy. In some embodiments, individuals with refractory NHL are identified using the response criteria disclosed in Cheson at al., 2014 (South Asian J Cancer. 2014 Jan-Mar; 3(1): 66-70). In that document, non-responders are defined as individuals where there is either (i) a >50% increase from nadir in the sum product of diameters of any previously identified abnormal node, or (ii) an appearance of any new lesion during or at the end of therapy. In some embodiments, individuals with refractory leukaemia are identified as individuals with either stable or progressive disease who have completed one complete treatment cycle, or individual achieving partial response after two or more complete treatment cycles.

The first target protein is preferably CD19 or CD22.

### Samples

The sample may comprise or may be derived from: a quantity of blood; a quantity of serum derived from the individuals blood which may comprise the fluid portion of the blood obtained after removal of the fibrin clot and blood cells; a quantity of pancreatic juice; a tissue sample or biopsy; or cells isolated from said individual.

A sample may be taken from any tissue or bodily fluid. In certain aspects, the sample may include or may be derived from a tissue sample, biopsy, resection or isolated cells from said individual.

In certain aspects, the sample is a tissue sample. The sample may be a sample of tumor tissue, such as cancerous tumor tissue. The sample may have been obtained by a tumor biopsy. In some aspects, the sample is a lymphoid tissue sample, such as a lymphoid lesion sample or lymph node biopsy. In some cases, the sample is a skin biopsy.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a individual’s blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of a first target polypeptide or nucleic acid in the blood, in white blood cells, peripheral blood mononuclear cells, granulocytes and/or red blood cells.

The sample may be fresh or archival. For example, archival tissue may be from the first diagnosis of an individual, or a biopsy at a relapse. In certain aspects, the sample is a fresh biopsy.

The first target polypeptide is preferably CD19 or CD22.

### Individual Status

The individual may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human

Furthermore, the individual may be any of its forms of development, for example, a foetus. In one preferred embodiment, the individual is a human. The terms “subject”, “patient” and “individual” are used interchangeably herein.

In some aspects disclosed herein, an individual has, or is suspected as having, or has been identified as being at risk of, cancer. In some aspects disclosed herein, the individual has already received a diagnosis of cancer. The individual may have received a diagnosis of non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL). [Fielding A., Haematologica. 2010 Jan; 95(1): 8-12].

In some cases, the individual has received a diagnosis of non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL). [Fielding A., Haematologica. 2010 Jan; 95(1): 8-12].

In some cases the individual has, is suspected of having, or has received a diagnosis of, a proliferative disease characterised by the presence of a neoplasm comprising both CD19+ve and CD19-ve cells. The neoplasm may be composed of CD19-ve neoplastic cells, optionally wherein the CD19-ve neoplastic cells are associated with CD19+ve neoplastic or non-neoplastic cells. The neoplasm or neoplastic cells may be all or part of a solid tumour. The solid tumor may be a neoplasm, including a non-haematological cancer, comprising or composed of CD19+ve neoplastic cells. In some cases the individual has, is suspected of having, or has received a diagnosis of, a proliferative disease characterised by the presence of a neoplasm comprising both CD22+ve and CD22-ve cells. The neoplasm may be composed of CD22-ve neoplastic cells, optionally wherein the CD22-ve neoplastic cells are associated with CD22+ve neoplastic or non-neoplastic cells. The neoplasm or neoplastic cells may be all or part of a solid tumour. The solid tumor may be a neoplasm, including a non-haematological cancer, comprising or composed of CD22+ve neoplastic cells

In some cases, the individual has, is suspected of having, or has received a diagnosis of a solid tumour.

“Solid tumor” herein will be understood to include solid haematological cancers such as lymphomas (Hodgkin’s lymphoma or non-Hodgkin’s lymphoma) which are discussed in more detail herein.

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 Jan;27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

In some cases, the individual has received a diagnosis of a solid cancer containing CD19+ or CD22+ expressing infiltrating cells.

The Individual may be undergoing, or have undergone, a therapeutic treatment for that cancer. The subject may, or may not, have previously received ADCX19 or ADCX22. In some cases the cancer is lymphoma, including non-Hodgkins lymphoma.

The Individual may be undergoing, or have undergone, treatment with an anti-CD20 agent. In some cases the individual may be refractory to treatment (or further treatment) with the anti-CD20 agent. In some cases the anti-CD20 agent may be Rituximab. In embodiments where the individual is undergoing, or has undergone, treatment with an anti-CD20 agent, the anti-CD19 ADC / secondary agent combination may be administered in combination with an anti-CD20 agent, or without continued administration of the anti-CD20 agent.

### Controls

In some aspects, target expression in the individual is compared to target expression in a control. Controls are useful to support the validity of staining, and to identify experimental artefacts.

In some cases, the control may be a reference sample or reference dataset. The reference may be a sample that has been previously obtained from a individual with a known degree of suitability. The reference may be a dataset obtained from analyzing a reference sample.

Controls may be positive controls in which the target molecule is known to be present, or expressed at high level, or negative controls in which the target molecule is known to be absent or expressed at low level.

Controls may be samples of tissue that are from individuals who are known to benefit from the treatment. The tissue may be of the same type as the sample being tested. For example, a sample of tumor tissue from a individual may be compared to a control sample of tumor tissue from a individual who is known to be suitable for the treatment, such as a individual who has previously responded to the treatment.

In some cases the control may be a sample obtained from the same individual as the test sample, but from a tissue known to be healthy. Thus, a sample of cancerous tissue from a individual may be compared to a non-cancerous tissue sample.

In some cases, the control is a cell culture sample.

In some cases, a test sample is analyzed prior to incubation with an antibody to determine the level of background staining inherent to that sample.

In some cases an isotype control is used. Isotype controls use an antibody of the same class as the target specific antibody, but are not immunoreactive with the sample. Such controls are useful for distinguishing non-specific interactions of the target specific antibody.

The methods may include hematopathologist interpretation of morphology and immunohistochemistry, to ensure accurate interpretation of test results. The method may involve confirmation that the pattern of expression correlates with the expected pattern. For example, where the amount of a first target protein and/or a second target protein expression is analyzed, the method may involve confirmation that in the test sample the expression is observed as membrane staining, with a cytoplasmic component. The method may involve confirmation that the ratio of target signal to noise is above a threshold level, thereby allowing clear discrimination between specific and non-specific background signals.

The first target protein is preferably CD19 or CD22.

### Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of an ADC and a secondary agent. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The ADC may comprise an anti-CD19 antibody or an anti-CD22 antibody. The anti-CD19 antibody may be RB4v1.2 antibody. The anti-CD22 antibody may be EMabC220.The ADC may comprise a drug which is a PBD dimer. The ADC may be an anti-CD19-ADC, and in particular, ADCX19. The ADC may be an anti-CD22-ADC, and in particular, ADCX22. The ADC may be an ADC disclosed in WO2014/057117 or WO2014/057122.

The secondary agent may be:
(a) a Bruton’s Tyrosine Kinase inhibitor (BTKi), such as Ibrutinib (Imbruvica), Acalabrutinib/ACP-196, ONO/GS-4059, Spebrutinib/AVL-292/CC-292, HM71224 (Poseltinib) or BGB-3111 (Zanubrutinib);
(b) a PD1 antagonist, such as pembrolizumab, nivolumab, MEDI0680, PDR001 (spartalizumab), Camrelizumab, AUNP12, Pidilizumab, Cemiplimab (REGN-2810), AMP-224, BGB-A317 (Tisleizumab), or BGB-108;
(c) a PD-L1 antagonist, such as atezolizumab (Tecentriq), BMS-936559/MDX-1105, durvalumab/MED14736, or MSB0010718C (Avelumab);
(d) a GITR (Glucocorticoid-Induced TNFR-Related protein) agonist, such as MEDI1873, TRX518, GWN323, MK-1248, MK-4166, BMS-986156 or INCAGN1876;
(e) an OX40 agonist, such as MEDI0562, MEDI6383, MOXR0916, RG7888, OX40mAb24, INCAGN1949, GSK3174998, or PF-04518600;
(f) a CTLA-4 antagonist, such as ipilimumab (brand name Yervoy) or Tremelimumab (Originally developed by Pfizer, now Medimmune);
(g) Fludarabine or Cytarabine;
(h) a hypomethylating agent, such as cytidine analogs - for example, 5-azacytidine (azacitidine) and 5-aza-2′-deoxycytidine (decitabine);
(i) an agent that upregulates HER2 expression, such as gemcitabine and tamoxifen; or
(j) an anti-CD20 agent, such as rituximab, obinutuzumab, Ibritumomab tiuxetan, tositumomab, Ofatumumab, Ocaratuzumab, Ocrelizumab, and Veltuzumab.

The treatment may involve administration of the ADC / secondary agent combination alone or in further combination with other treatments, either simultaneously or sequentially dependent upon the condition to be treated.

An example method of treatment involves:
(1) identifying an individual has been treated with, or is being treated with an anti-CD20 agent, such as Rituximab;
(2) administering to the individual an anti-CD19 ADC, such as ADCx19, optionally in combination with a secondary agent; and, optionally
(3) administering to the individual an anti-CD20 agent, such as Rituximab in combination with the anti-CD19 ADC and/or secondary agent (for example, at the same time as the ADC, or after the ADC).

Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: Lenalidomide (REVLIMID®, Celgene), Vorinostat (ZOLINZA®, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDA®, Mundipharma International), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo- 2,3,4,6,8-pentazabicyclo [4.3.0] nona-2,7,9-triene- 9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-½, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™(Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1l, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2’,2”-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above. Combinations of agents may be used, such as CHP (doxorubicin, prednisone, cyclophosphamide), or CHOP (doxorubicin, prednisone, cyclophopsphamide, vincristine).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), pertuzumab (PERJETA™, OMNITARG™, 2C4, Genentech), trastuzumab (HERCEPTIN®, Genentech), MDX-060 (Medarex) and the antibody drug conjugate, gemtuzumab ozogamicin (MYLOTARGO, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Compositions according to the present disclosure are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer’s Injection, Lactated Ringer’s Injection. Preservatives, stabilisers, buffers, antioxidants and/or other additives may be included, as required.

### Dosage

It will be appreciated by one of skill in the art that appropriate dosages of the ADC, secondary agent, and/or the anti-CD20 agent, and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of ADC is determined by the expression of a first target protein observed in a sample obtained from the subject. Thus, the level or localisation of expression of the first target protein in the sample may be indicative that a higher or lower dose of ADC is required. For example, a high expression level of the first target protein may indicate that a higher dose of ADC would be suitable. In some cases, a high expression level of the first target protein may indicate the need for administration of another agent in addition to the ADC. For example, administration of the ADC in conjunction with a chemotherapeutic agent. A high expression level of the first target protein may indicate a more aggressive therapy.

In certain aspects, the dosage of the secondary agent is determined by the expression of a second target protein observed in a sample obtained from the subject. Thus, the level or localisation of expression of the second target protein in the sample may be indicative that a higher or lower dose of secondary agent is required. For example, a high expression level of the second target protein may indicate that a higher dose of secondary agent would be suitable. In some cases, a high expression level of the second target protein may indicate the need for administration of another agent in addition to the secondary agent. For example, administration of the secondary agent in conjunction with a chemotherapeutic agent. A high expression level of the second target protein may indicate a more aggressive therapy.

In certain aspects, the dosage of the anti-CD20 agent is determined by the expression of CD20 observed in a sample obtained from the subject. Thus, the level or localisation of expression of CD20 in the sample may be indicative that a higher or lower dose of anti-CD20 agent is required. For example, a high expression level of CD20 may indicate that a higher dose of anti-CD20 agent would be suitable. In some cases, a high expression level of CD20 may indicate the need for administration of another agent in addition to the anti-CD20 agent. For example, administration of the anti-CD20 agent in conjunction with a chemotherapeutic agent. A high expression level of CD20 may indicate a more aggressive therapy.

In certain aspects, the dosage level is determined by the expression of a first target protein on neoplastic cells in a sample obtained from the subject. For example, when the target neoplasm is composed of, or comprises, neoplastic cells expressing the first target protein.

In certain aspects, the dosage level is determined by the expression of a first target protein on cells associated with the target neoplasm. For example, the target neoplasm may be a solid tumour composed of, or comprising, neoplastic cells that express the first target protein. For example, the target neoplasm may be a solid tumour composed of, or comprising, neoplastic cells that do not express the first target protein. The cells expressing the first target protein may be neoplastic or non-neoplastic cells associated with the target neoplasm.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 µg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, each active compound is administered to a human subject according to the following dosage regime: about 100 mg, 3 times daily.

In one embodiment, each active compound is administered to a human subject according to the following dosage regime: about 150 mg, 2 times daily.

In one embodiment, each active compound is administered to a human subject according to the following dosage regime: about 200 mg, 2 times daily.

However in one embodiment, each conjugate compound is administered to a human subject according to the following dosage regime: about 50 or about 75 mg, 3 or 4 times daily.

In one embodiment, each conjugate compound is administered to a human subject according to the following dosage regime: about 100 or about 125 mg, 2 times daily.

For the ADC, where it is a PBD bearing ADC, the dosage amounts described above may apply to the conjugate (including the PBD moiety and the linker to the antibody) or to the effective amount of PBD compound provided, for example the amount of compound that is releasable after cleavage of the linker.

The first target protein is preferably CD19 or CD22. The ADC may comprise an anti-CD19 antibody or an anti-CD22 antibody. The anti-CD19 antibody may be RB4v1.2 antibody. The anti-CD22 antibody may be EMabC220.The ADC may comprise a drug which is a PBD dimer. The ADC may be an anti-CD19-ADC, and in particular, ADCX19. The ADC may be an anti-CD22-ADC, and in particular, ADCX22. The ADC may be an ADC disclosed in WO2014/057117 or WO2014/057122.

The secondary agent may be Fludarabine or Cytarabine.

The anti-CD20 agent may be an anti-CD20 antibody or antibody-conjugate. Suitable anti-CD20 antibodies or antibody-conjugates include rituximab, obinutuzumab, Ibritumomab tiuxetan, tositumomab, Ofatumumab, Ocaratuzumab, Ocrelizumab, and Veltuzumab. Preferably the anti-CD20 agent is rituximab.

### Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, US 4816567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (US 4816567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, ү, and µ, respectively. The subunit structures and three-dimensional configurations of different classes of immunoglobulins are well known.

### BRIEF DESCRIPTION OF THE FIGURES

Embodiments and experiments illustrating the principles of the disclosure will now be discussed with reference to the accompanying figures in which:

FIGS. 1A-1C. Sequences

FIG. 2. In vitro synergy of ADCx19 and Rituximab

FIG. 3. In vitro synergy of ADCx19 and Cytarabine

FIG. 4. In vitro synergy of ADCx22/Cytarabine (A) and ADCx22/Fludarabine (B)

FIG. 5. In vivo synergy of ADCx19 / Cytarabine (A) and ADCx19/Rituximab(B); single group data from 5B is shown in FIG. 5C

FIG. 6. In vitro synergy in CD19+ve Ramos cell line of ADCx19 with each of Cvtarabine (6A), Decitabine (6B), Gemcitabine (6C), and Fludarabine (6D)

FIG. 7. In vitro synergy in CD22+ve Ramos cell line of ADCx22 with each of Cvtarabine (7A), Decitabine (7B), Gemcitabine (7C), and Fludarabine (7D)

FIG. 8. In vitro cytotoxicity data against the Karpas299 cell line of Cvtarabine alone (top graph) or in combination with ADCx25 (bottom graph).

FIG. 9. In vitro cytotoxicity data against the KG-1 cell line of cytarabine alone (top graph) or in combination with ADCx25 (bottom graph).

FIG. 10. Surface CD25 levels on the Karpas299 cell line (left graph) and KG-1 cell line (right graph) incubated with or without Cytarabine.

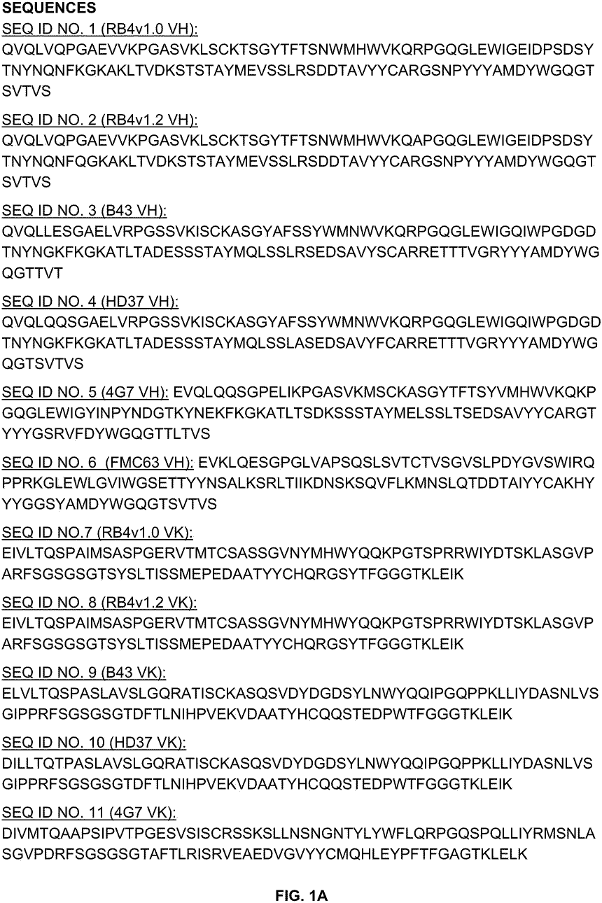

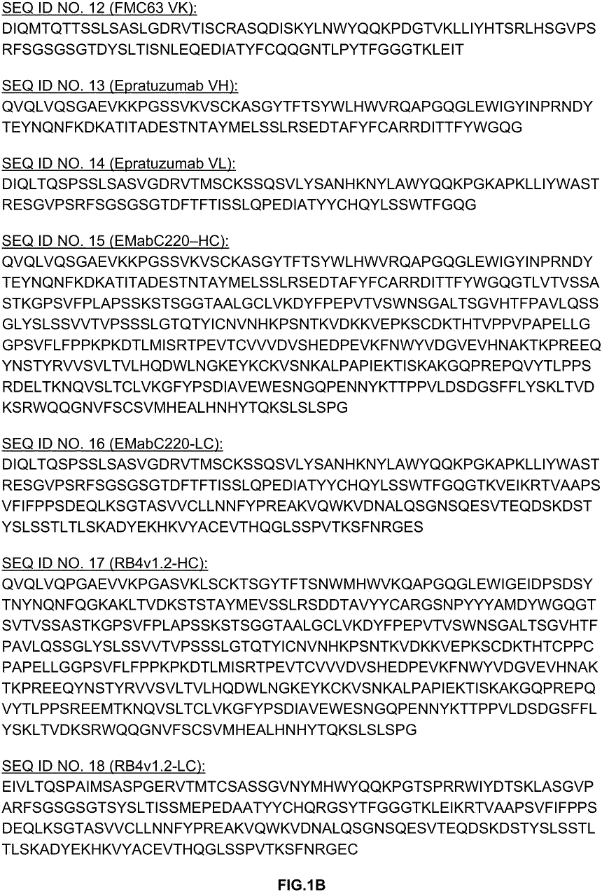

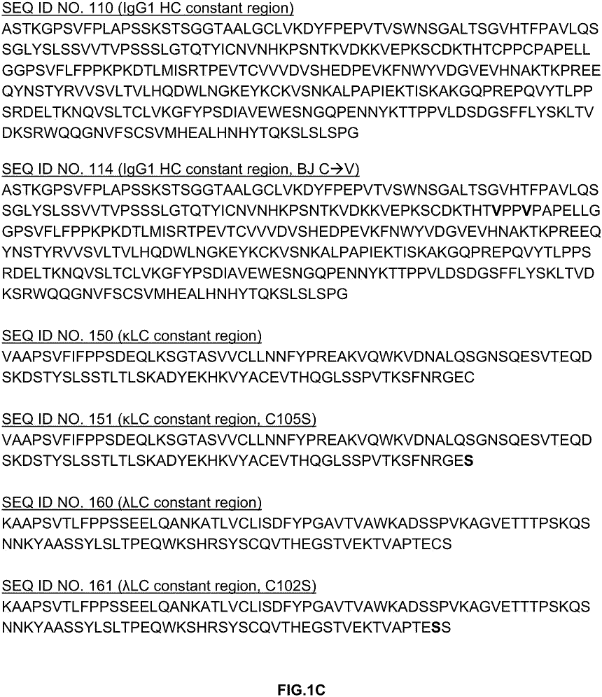

The disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### SOME EMBODIMENTS

The following paragraphs describe some specific embodiments of the present disclosure:

1. A method of selecting an individual as suitable for treatment with ADCx19, optionally in combination with a secondary agent, wherein the individual is selected for treatment if the individual has been treated with an anti-CD20 agent.

2. A method of selecting an individual as suitable for treatment with ADCx19, optionally in combination with a secondary agent, wherein the individual is selected for treatment if the individual is being treated with an anti-CD20 agent.

3. The method according to any one of the preceding paragraphs, wherein the individual is selected for treatment if the individual is refractory to treatment, or further treatment, with an anti-CD20 agent.

4. A method for treating a disorder in an individual, the method comprising:
(i) selecting an individual as suitable for treatment by a method according to any one of paragraphs 1 to 3; and
(ii) administering to the individual an effective amount of ADCx19, optionally in combination with a secondary agent.

5. The method according to paragraph 4, further comprising administering an anti-CD20 agent in combination with ADCx19, optionally in further combination with a secondary agent.

6. A method for treating a disorder in an individual, the method comprising administering to the individual an effective amount of:
ADCx19; and
a secondary agent;
optionally in further combination with an anti-CD20 agent.

7. The method according to paragraph 6, wherein the individual is selected for treatment according to a method according to any one of paragraphs 1 to 3.

8. The method according to any one of paragraphs 5 to 7, wherein the treatment comprises administering ADCx19, optionally in combination with a secondary agent, before an anti-CD20 agent, simultaneous with an anti-CD20 agent, or after an anti-CD20 agent.

9. The method according to any previous paragraph, wherein the treatment further comprises administering a chemotherapeutic agent.

10. The method according to any previous paragraph, wherein the individual is human.

11. The method according to any preceding paragraph, wherein the individual has a disorder or has been determined to have a disorder.

12. The method according to paragraph 11, wherein the individual has, or has been has been determined to have, a cancer which expresses CD19 or CD19+ tumour-associated non-tumour cells, such as CD19+ infiltrating cells.

13. The method according to any previous paragraph, wherein the individual is undergoing treatment with an anti-CD20 agent.

14. The method according to any previous paragraph, wherein the individual has undergone treatment with an anti-CD20 agent.

15. The method according to any previous paragraph, wherein the individual is refractory to treatment, or further treatment, with an anti-CD20 agent.

16. The method according to any one of the preceding paragraphs, wherein the treatment has increased efficacy as compared to monotherapy with ADCx19, a secondary agent, or an anti-CD20 agent alone, or combinations of ADCx19/Cytarabine, ADCx19/Fludarabine, ADCx19/an anti-CD20 agent, Cytarabine/an anti-CD20 agent, or FLudarabine/an anti-CD20 agent.

17. The method according to any previous paragraph, wherein the disorder is a proliferative disease.

18. The method of paragraph 17, wherein the disorder is cancer.

19. The method of paragraph 18, wherein the disorder is selected from the group comprising: non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL).

20. The method of paragraph 18, wherein the disorder is characterized by the presence of one or more solid tumours.

21. The method of paragraph 20, wherein the solid tumour is pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, or head and neck cancer.

22. ADCx19, optionally in combination with a secondary agent, for use in a method of treatment according to any one of paragraphs 4 to 21.

23. A composition comprising ADCx19, optionally in combination with a secondary agent, for use in a method of treatment according to any one of paragraphs 4 to 21.

24. an anti-CD20 agent for use in a method of treatment according to any one of paragraphs 5 to 21.

25. A composition comprising an anti-CD20 agent, for use in a method of treatment according to any one of paragraphs 5 to 21.

26. Use of ADCx19, optionally in combination with a secondary agent, in the manufacture of a medicament for treating a disorder in an individual, wherein the treatment comprises the method of any one of paragraphs 4 to 21.

27. Use of an anti-CD20 agent in the manufacture of a medicament for treating a disorder in an individual, wherein the treatment comprises the method of any one of paragraphs 5 to 21.

28. A kit comprising:
a first medicament comprising ADCx19;
optionally, a second medicament comprising a secondary agent;
a package insert comprising instructions for administration of the first medicament according to the method of any one or paragraphs 4 to 21.

29. The kit according to paragraph 28, further comprising:
a third medicament comprising an anti-CD20 agent.

30. A composition, method, use, or kit according to any one of the preceding paragraphs, wherein the secondary agent is a Bruton’s Tyrosine Kinase inhibitor (BTKi).

31. A composition, method, use, or kit according to paragraph 30, wherein the Bruton’s Tyrosine Kinase inhibitor (BTKi) is selected from Ibrutinib (Imbruvica), Acalabrutinib/ACP-196, ONO/GS-4059, Spebrutinib/AVL-292/CC-292, HM71224 (Poseltinib) and BGB-3111 (Zanubrutinib).

32. A composition, method, use, or kit according to any one of paragraphs 1 to 29, wherein the secondary agent is a PD1 antagonist.

33. A composition, method, use, or kit according to paragraph 32, wherein the PD1 antagonist is selected from pembrolizumab, nivolumab, MEDI0680, PDR001 (spartalizumab), Camrelizumab, AUNP12, Pidilizumab Cemiplimab (REGN-2810), AMP-224, BGB-A317 (Tisleizumab), and BGB-108.

34. A composition, method, use, or kit according to any one of paragraphs 1 to 29, wherein the secondary agent is a PD-L1 antagonist.

35. A composition, method, use, or kit according to paragraph 34, wherein the PD-L1 antagonist is selected from atezolizumab (Tecentriq), BMS-936559/MDX-1105, durvalumab/MEDI4736, and MSB0010718C (Avelumab).

36. A composition, method, use, or kit according to any one of paragraphs 1 to 29, wherein the secondary agent is a GITR (Glucocorticoid-Induced TNFR-Related protein) agonist.

37. A composition, method, use, or kit according to paragraph 36, wherein the GITR (Glucocorticoid-Induced TNFR-Related protein) agonist is selected from MEDl1873, TRX518, GWN323, MK-1248, MK4166, BMS-986156 and INCAGN1876.

38. A composition, method, use, or kit according to any one of paragraphs 1 to 29, wherein the secondary agent is a OX40 agonist.

39. A composition, method, use, or kit according to paragraph 38, wherein the OX40 agonist is selected from MEDl0562, MEDl6383, MOXR0916, RG7888, OX40mAb24, INCAGN1949, GSK3174998, and PF-04518600.

40. A composition, method, use, or kit according to any one of paragraphs 1 to 29, wherein the secondary agent is a CTLA-4 antagonist.

41. A composition, method, use, or kit according to paragraph 40, wherein the CTLA-4 antagonist is selected from ipilimumab and Tremelimumab.

42. A composition, method, use, or kit according to any one of paragraphs 1 to 29, wherein the secondary agent is Cytarabine.

43. A composition, method, use, or kit according to any one of paragraphs 1 to 29, wherein the secondary agent is Fludarabine.

44. A composition, method, use, or kit according to any one of paragraphs 1 to 29, wherein the secondary agent is a hypomethylating agent.

45. A composition, method, use, or kit according to paragraph 44, wherein the hypomethylating agent is selected from 5-azacytidine (azacitidine) and 5-aza-2’-deoxycytidine (decitabine).

46. A composition, method, use, or kit according to any one of paragraphs 1 to 29, wherein the secondary agent is an agent that upregulates HER2 expression.

47. A composition, method, use, or kit according to paragraph 46, wherein the agent that upregulates HER2 expression is selected from gemcitabine and tamoxifen.

48. A composition, method, use, or kit according to any preceding paragraph, wherein the anti-CD20 agent is rituximab.

49. A composition, method, use, or kit according to any preceding paragraph, wherein the anti-CD20 agent is selected from obinutuzumab, Ibritumomab tiuxetan, tositumomab, Ofatumumab, Ocaratuzumab, Ocrelizumab, and Veltuzumab.

1x. A method of selecting an individual as suitable for treatment with ADCx22, optionally in combination with a secondary agent, wherein the individual is selected for treatment if the individual has been treated with an anti-CD20 agent.

2x. A method of selecting an individual as suitable for treatment with ADCx22, optionally in combination with a secondary agent, wherein the individual is selected for treatment if the individual is being treated with an anti-CD20 agent.

3x. The method according to any one of the preceding paragraphs, wherein the individual is selected for treatment if the individual is refractory to treatment, or further treatment, with an anti-CD20 agent.

4x. A method for treating a disorder in an individual, the method comprising:
(i) selecting an individual as suitable for treatment by a method according to any one of paragraphs 1x to 3x; and
(ii) administering to the individual an effective amount of ADCx22, optionally in combination with a secondary agent.

5x. The method according to paragraph 4x, further comprising administering an anti-CD20 agent in combination with ADCx22, optionally in further combination with a secondary agent.

6x. A method for treating a disorder in an individual, the method comprising administering to the individual an effective amount of:
ADCx22; and
a secondary agent;
optionally in further combination with an anti-CD20 agent.

7x. The method according to paragraph 6x, wherein the individual is selected for treatment according to a method according to any one of paragraphs 1x to 3x.

8x. The method according to any one of paragraphs 5x to 7x, wherein the treatment comprises administering ADCx22, optionally in combination with a secondary agent, before an anti-CD20 agent, simultaneous with an anti-CD20 agent, or after an anti-CD20 agent.

9x. The method according to any previous paragraph, wherein the treatment further comprises administering a chemotherapeutic agent.

10x. The method according to any previous paragraph, wherein the individual is human.

11x. The method according to any preceding paragraph, wherein the individual has a disorder or has been determined to have a disorder.

12x. The method according to paragraph 11x, wherein the individual has, or has been has been determined to have, a cancer which expresses CD22+ tumour-associated non-tumour cells, such as CD22+ infiltrating cells.

13x. The method according to any previous paragraph, wherein the individual is undergoing treatment with an anti-CD20 agent.

14x. The method according to any previous paragraph, wherein the individual has undergone treatment with an anti-CD20 agent.

15x. The method according to any previous paragraph, wherein the individual is refractory to treatment, or further treatment, with an anti-CD20 agent.

16x. The method according to any one of the preceding paragraphs, wherein the treatment has increased efficacy as compared to monotherapy with ADCx22, a secondary agent, or an anti-CD20 agent alone, or combinations of ADCx22/Cytarabine, ADCx22/Fludarabine, ADCx22/an anti-CD20 agent, Cytarabine/an anti-CD20 agent, or FLudarabine/an anti-CD20 agent.

17x. The method according to any previous paragraph, wherein the disorder is a proliferative disease.

18x. The method of paragraph 17x, wherein the disorder is cancer.

19x. The method of paragraph 18x, wherein the disorder is selected from the group comprising: non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL).

20x. The method of paragraph 18x, wherein the disorder is characterized by the presence of one or more solid tumours.

21x. The method of paragraph 20x, wherein the solid tumour is pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, or head and neck cancer.

22x. ADCx22, optionally in combination with a secondary agent, for use in a method of treatment according to any one of paragraphs 4x to 21x.

23x. A composition comprising ADCx22, optionally in combination with a secondary agent, for use in a method of treatment according to any one of paragraphs 4x to 21x.

24x. an anti-CD20 agent for use in a method of treatment according to any one of paragraphs 5x to 21x.

25x. A composition comprising an anti-CD20 agent, for use in a method of treatment according to any one of paragraphs 5x to 21x.

26x. Use of ADCx22, optionally in combination with a secondary agent, in the manufacture of a medicament for treating a disorder in an individual, wherein the treatment comprises the method of any one of paragraphs 4x to 21x.

27x. Use of an anti-CD20 agent in the manufacture of a medicament for treating a disorder in an individual, wherein the treatment comprises the method of any one of paragraphs 5x to 21x.

28x. A kit comprising:
a first medicament comprising ADCx22;
optionally, a second medicament comprising a secondary agent;
a package insert comprising instructions for administration of the first medicament according to the method of any one or paragraphs 4x to 21x.

29x. The kit according to paragraph 28x, further comprising:
a third medicament comprising an anti-CD20 agent.

30x. A composition, method, use, or kit according to any one of the preceding paragraphs, wherein the secondary agent is a Bruton’s Tyrosine Kinase inhibitor (BTKi).

31x. A composition, method, use, or kit according to paragraph 30x, wherein the Bruton’s Tyrosine Kinase inhibitor (BTKi) is selected from Ibrutinib (Imbruvica), Acalabrutinib/ACP-196, ONO/GS-4059, Spebrutinib/AVL-292/CC-292, HM71224 (Poseltinib) and BGB-3111 (Zanubrutinib).

32x. A composition, method, use, or kit according to any one of paragraphs 1x to 29x, wherein the secondary agent is a PD1 antagonist.

33x. A composition, method, use, or kit according to paragraph 32x, wherein the PD1 antagonist is selected from pembrolizumab, nivolumab, MEDI0680, PDR001 (spartalizumab), Camrelizumab, AUNP12, Pidilizumab Cemiplimab (REGN-2810), AMP-224, BGB-A317 (Tisleizumab), and BGB-108.

34x. A composition, method, use, or kit according to any one of paragraphs 1x to 29x, wherein the secondary agent is a PD-L1 antagonist.

35x. A composition, method, use, or kit according to paragraph 34x, wherein the PD-L1 antagonist is selected from atezolizumab (Tecentriq), BMS-936559/MDX-1105, durvalumab/MEDI4736, and MSB0010718C (Avelumab).

36x. A composition, method, use, or kit according to any one of paragraphs 1x to 29x, wherein the secondary agent is a GITR (Glucocorticoid-Induced TNFR-Related protein) agonist.

37x. A composition, method, use, or kit according to paragraph 36x, wherein the GITR (Glucocorticoid-Induced TNFR-Related protein) agonist is selected from MEDl1873, TRX518, GWN323, MK-1248, MK4166, BMS-986156 and INCAGN1876.

38x. A composition, method, use, or kit according to any one of paragraphs 1x to 29x, wherein the secondary agent is a OX40 agonist.

39x. A composition, method, use, or kit according to paragraph 38x, wherein the OX40 agonist is selected from MEDl0562, MEDl6383, MOXR0916, RG7888, OX40mAb24, INCAGN1949, GSK3174998, and PF-04518600.

40x. A composition, method, use, or kit according to any one of paragraphs 1x to 29x, wherein the secondary agent is a CTLA-4 antagonist.

41x. A composition, method, use, or kit according to paragraph 40x, wherein the CTLA-4 antagonist is selected from ipilimumab and Tremelimumab.

42x. A composition, method, use, or kit according to any one of paragraphs 1x to 29x, wherein the secondary agent is Cytarabine.

43x. A composition, method, use, or kit according to any one of paragraphs 1x to 29x, wherein the secondary agent is Fludarabine.

44x. A composition, method, use, or kit according to any one of paragraphs 1x to 29x, wherein the secondary agent is a hypomethylating agent.

45x. A composition, method, use, or kit according to paragraph 44x, wherein the hypomethylating agent is selected from 5-azacytidine (azacitidine) and 5-aza-2’-deoxycytidine (decitabine).

46x. A composition, method, use, or kit according to any one of paragraphs 1x to 29x, wherein the secondary agent is an agent that upregulates HER2 expression.

47x. A composition, method, use, or kit according to paragraph 46x, wherein the agent that upregulates HER2 expression is selected from gemcitabine and tamoxifen.

48x. A composition, method, use, or kit according to any preceding paragraph, wherein the anti-CD20 agent is rituximab.

49x. A composition, method, use, or kit according to any preceding paragraph, wherein the anti-CD20 agent is selected from obinutuzumab, Ibritumomab tiuxetan, tositumomab, Ofatumumab, Ocaratuzumab, Ocrelizumab, and Veltuzumab.

1a. A method for treating cancer in an individual, the method comprising administering to the individual an effective amount of ADCX19, a secondary agent, and optionally an anti-CD20 agent.

2a. A first composition comprising ADCX19 for use in a method of treating cancer in an individual, wherein the treatment comprises administration of the first composition in combination with a second composition comprising a secondary agent, and optionally in combination with a third composition comprising an anti-CD20 agent.

3a. A first composition comprising a secondary agent for use in a method of treating a disorder in an individual, wherein the treatment comprises administration of the first composition in combination with a second composition comprising ADCX19, and optionally in combination with a third composition comprising an anti-CD20 agent.

4a. Use of ADCX19 in the manufacture of a medicament for treating cancer in an individual, wherein the medicament comprises ADCX19, and wherein the treatment comprises administration of the medicament in combination with a composition comprising a secondary agent, and optionally in further combination with an anti-CD20 agent.

5a. Use of A secondary agent in the manufacture of a medicament for treating cancer in an individual, wherein the treatment comprises administration of the medicament in combination with a composition comprising ADCX19, and optionally in further combination with a compostion comprising an anti-CD20 agent.

6a. A kit comprising:
a first medicament comprising ADCX19;
a second medicament comprising a secondary agent;
optionally, a third medicament comprising an anti-CD20 agent; and, further optionally,
a package insert comprising instructions for administration of the first medicament to an individual in combination with the second medicament, and optionally the third medicament if present, for the treatment of cancer.

7a. A kit comprising a medicament comprising ADCX19 and a package insert comprising instructions for administration of the medicament to an individual in combination with a composition comprising a secondary agent, and optionally in further combination with a composition comprising an anti-CD20 agent, for the treatment of cancer.

8a. A kit comprising a medicament comprising a secondary agent and a package insert comprising instructions for administration of the medicament to an individual in combination with a composition comprising ADCX19, and optionally in further combination with a composition comprising an anti-CD20 agent, for the treatment of cancer.

9a. A pharmaceutical composition comprising ADCX19,a secondary agent, and optionally an anti-CD20 agent.

10a. A method of treating cancer in an individual, the method comprising administering to the individual an effective amount of the composition of paragraph 9.

11a. The composition of paragraph 9 for use in a method of treating cancer in an individual.

12a. The use of the composition of paragraph 9 in the manufacture of a medicament for treating cancer in an individual.

13a. A kit comprising the composition of paragraph 9 and a set of instructions for administration of the medicament to an individual for the treatment of cancer.

14a. The composition, method, use, or kit according to any previous paragraph, wherein the treatment comprises administering ADCX19 and a secondary agent before the an anti-CD20 agent, simultaneous with the an anti-CD20 agent, or after the an anti-CD20 agent.

15a. The composition, method, use, or kit according to any previous paragraph, wherein the treatment further comprises administering a chemotherapeutic agent.

16a. The composition, method, use, or kit according to any previous paragraph, wherein the individual is human.

17a. The composition, method, use, or kit according to any previous paragraph, wherein the individual has a disorder or has been determined to have cancer.

18a. The composition, method, use, or kit according any previous paragraph, wherein the individual has, or has been has been determined to have, a cancer characterised by the presence of a neoplasm comprising both CD19+ve and CD19-ve cells.

19a. The composition, method, use, or kit according any previous paragraph, wherein the individual has, or has been has been determined to have, a cancer characterised by the presence of a neoplasm comprising, or composed of, CD19-ve neoplastic cells.

20a. The composition, method, use, or kit according to any previous paragraph, wherein the cancer or neoplasm is all or part of a solid tumour.

21a. The composition, method, use, or kit according to any previous paragraph, wherein the individual has, or has been has been determined to have, a cancer which expresses CD19 or CD19+ tumour-associated non-tumour cells, such as CD19+ infiltrating cells.

22a. The composition, method, use, or kit according to any one of the preceding paragraphs, wherein the treatment:
a) effectively treats a broader range of disorders,
b) effectively treats resistant, refractory, or relapsed disorders,
c) has an increased response rate, and / or
d) has increased durability; as compared to treatment with either ADCX19 or the a secondary agent alone.

23a. The composition, method, use, or kit according to any one of the preceding paragraphs, wherein the cancer is selected from the group comprising: Hodgkin’s and non-Hodgkin’s Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL), leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph-ALL), and solid tumours, such solid tumours of pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, or head and neck cancer.

24a. A composition, method, use, or kit according to any one of the preceding paragraphs, wherein the secondary agent is a Bruton’s Tyrosine Kinase inhibitor (BTKi).

25a. A composition, method, use, or kit according to paragraph 24a, wherein the Bruton’s Tyrosine Kinase inhibitor (BTKi) is selected from Ibrutinib (Imbruvica), Acalabrutinib/ACP-196, ONO/GS-4059, Spebrutinib/AVL-292/CC-292, HM71224 (Poseltinib) and BGB-3111 (Zanubrutinib).

26a. A composition, method, use, or kit according to any one of paragraphs 1a to 23a, wherein the secondary agent is a PD1 antagonist.

27a. A composition, method, use, or kit according to paragraph 26a, wherein the PD1 antagonist is selected from pembrolizumab, nivolumab, MEDI0680, PDR001 (spartalizumab), Camrelizumab, AUNP12, Pidilizumab Cemiplimab (REGN-2810), AMP-224, BGB-A317 (Tisleizumab), and BGB-108.

28a. A composition, method, use, or kit according to any one of paragraphs 1a to 23a, wherein the secondary agent is a PD-L1 antagonist.

29a. A composition, method, use, or kit according to paragraph 28a, wherein the PD-L1 antagonist is selected from atezolizumab (Tecentriq), BMS-936559/MDX-1105, durvalumab/MEDI4736, and MSB0010718C (Avelumab).

30a. A composition, method, use, or kit according to any one of paragraphs 1a to 23a, wherein the secondary agent is a GITR (Glucocorticoid-Induced TNFR-Related protein) agonist.

31a. A composition, method, use, or kit according to paragraph 30a, wherein the GITR (Glucocorticoid-Induced TNFR-Related protein) agonist is selected from MEDl1873, TRX518, GWN323, MK-1248, MK4166, BMS-986156 and INCAGN1876.

32a. A composition, method, use, or kit according to any one of paragraphs 1a to 23a, wherein the secondary agent is a OX40 agonist.

33a. A composition, method, use, or kit according to paragraph 32a, wherein the OX40 agonist is selected from MEDl0562, MEDl6383, MOXR0916, RG7888, OX40mAb24, INCAGN1949, GSK3174998, and PF-04518600.

34a. A composition, method, use, or kit according to any one of paragraphs 1a to 23a, wherein the secondary agent is a CTLA-4 antagonist.

35a. A composition, method, use, or kit according to paragraph 34a, wherein the CTLA-4 antagonist is selected from ipilimumab and Tremelimumab.

36a. A composition, method, use, or kit according to any one of paragraphs 1a to 23a, wherein the secondary agent is Cytarabine.

37a. A composition, method, use, or kit according to any one of paragraphs 1a to 23a, wherein the secondary agent is Fludarabine.

38a. A composition, method, use, or kit according to any one of paragraphs 1a to 23a, wherein the secondary agent is a hypomethylating agent.

39a. A composition, method, use, or kit according to paragraph 38a, wherein the hypomethylating agent is selected from 5-azacytidine (azacitidine) and 5-aza-2’-deoxycytidine (decitabine).

40a. A composition, method, use, or kit according to any one of paragraphs 1a to 23a, wherein the secondary agent is an agent that upregulates HER2 expression.

41a. A composition, method, use, or kit according to paragraph 40a, wherein the agent that upregulates HER2 expression is selected from gemcitabine and tamoxifen.

42a. A composition, method, use, or kit according to any preceding paragraph, wherein the anti-CD20 agent is rituximab.

43a. A composition, method, use, or kit according to any preceding paragraph, wherein the anti-CD20 agent is selected from obinutuzumab, Ibritumomab tiuxetan, tositumomab, Ofatumumab, Ocaratuzumab, Ocrelizumab, and Veltuzumab.

1b. A method for treating cancer in an individual, the method comprising administering to the individual an effective amount of ADCX22, a secondary agent, and optionally an anti-CD20 agent.

2b. A first composition comprising ADCX22 for use in a method of treating cancer in an individual, wherein the treatment comprises administration of the first composition in combination with a second composition comprising a secondary agent, and optionally in combination with a third composition comprising an anti-CD20 agent.

3b. A first composition comprising a secondary agent for use in a method of treating a disorder in an individual, wherein the treatment comprises administration of the first composition in combination with a second composition comprising ADCX22, and optionally in combination with a third composition comprising an anti-CD20 agent.

4b. Use of ADCX22 in the manufacture of a medicament for treating cancer in an individual, wherein the medicament comprises ADCX22, and wherein the treatment comprises administration of the medicament in combination with a composition comprising a secondary agent, and optionally in further combination with an anti-CD20 agent.

5b. Use of A secondary agent in the manufacture of a medicament for treating cancer in an individual, wherein the treatment comprises administration of the medicament in combination with a composition comprising ADCX22, and optionally in further combination with a compostion comprising an anti-CD20 agent.

6b. A kit comprising:
a first medicament comprising ADCX22;
a second medicament comprising a secondary agent;
optionally, a third medicament comprising an anti-CD20 agent; and, further optionally,
a package insert comprising instructions for administration of the first medicament to an individual in combination with the second medicament, and optionally the third medicament if present, for the treatment of cancer.

7b. A kit comprising a medicament comprising ADCX22 and a package insert comprising instructions for administration of the medicament to an individual in combination with a composition comprising a secondary agent, and optionally in further combination with a composition comprising an anti-CD20 agent, for the treatment of cancer.

8b. A kit comprising a medicament comprising a secondary agent and a package insert comprising instructions for administration of the medicament to an individual in combination with a composition comprising ADCX22, and optionally in further combination with a composition comprising an anti-CD20 agent, for the treatment of cancer.

9b. A pharmaceutical composition comprising ADCX22,a secondary agent, and optionally an anti-CD20 agent.

10b. A method of treating cancer in an individual, the method comprising administering to the individual an effective amount of the composition of paragraph 9.

11b. The composition of paragraph 9 for use in a method of treating cancer in an individual.

12b. The use of the composition of paragraph 9 in the manufacture of a medicament for treating cancer in an individual.

13b. A kit comprising the composition of paragraph 9 and a set of instructions for administration of the medicament to an individual for the treatment of cancer.

14b. The composition, method, use, or kit according to any previous paragraph, wherein the treatment comprises administering ADCX22 and a secondary agent before the an anti-CD20 agent, simultaneous with the an anti-CD20 agent, or after the an anti-CD20 agent.

15b. The composition, method, use, or kit according to any previous paragraph, wherein the treatment further comprises administering a chemotherapeutic agent.

16b. The composition, method, use, or kit according to any previous paragraph, wherein the individual is human.

17b. The composition, method, use, or kit according to any previous paragraph, wherein the individual has a disorder or has been determined to have cancer.

18b. The composition, method, use, or kit according any previous paragraph, wherein the individual has, or has been has been determined to have, a cancer characterised by the presence of a neoplasm comprising both CD22+ve and CD22-ve cells.

19b. The composition, method, use, or kit according any previous paragraph, wherein the individual has, or has been has been determined to have, a cancer characterised by the presence of a neoplasm comprising, or composed of, CD22-ve neoplastic cells.

20b. The composition, method, use, or kit according to any previous paragraph, wherein the cancer or neoplasm is all or part of a solid tumour.

21b. The composition, method, use, or kit according to any previous paragraph, wherein the individual has, or has been has been determined to have, a cancer which expresses CD22+ tumour-associated non-tumour cells, such as CD22+ infiltrating cells.

22b. The composition, method, use, or kit according to any one of the preceding paragraphs, wherein the treatment:
a) effectively treats a broader range of disorders,
b) effectively treats resistant, refractory, or relapsed disorders,
c) has an increased response rate, and / or
d) has increased durability; as compared to treatment with either ADCX22 or the a secondary agent alone.

23b. The composition, method, use, or kit according to any one of the preceding paragraphs, wherein the cancer is selected from the group comprising: Hodgkin’s and non-Hodgkin’s Lymphomb, including diffuse large B-cell lymphoma (DLBCL), follicular lymphomb, (FL), Mantle Cell lymphoma (MCL), chronic lymphatic lymphoma (CLL), Marginal Zone B-cell lymphoma (MZBL), leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph-ALL), and solid tumours, such solid tumours of pancreatic cancer, breast cancer, colorectal cancer, gastric and oesophageal cancer, leukemia and lymphomb, melanomb, non-small cell lung cancer, ovarian cancer, hepatocellular carcinomb, renal cell carcinomb, or head and neck cancer.

24b. A composition, method, use, or kit according to any one of the preceding paragraphs, wherein the secondary agent is a Bruton’s Tyrosine Kinase inhibitor (BTKi).

25b. A composition, method, use, or kit according to paragraph 24b, wherein the Bruton’s Tyrosine Kinase inhibitor (BTKi) is selected from Ibrutinib (Imbruvica), Acalabrutinib/ACP-196, ONO/GS-4059, Spebrutinib/AVL-292/CC-292, HM71224 (Poseltinib) and BGB-3111 (Zanubrutinib).

26b. A composition, method, use, or kit according to any one of paragraphs 1b to 23b, wherein the secondary agent is a PD1 antagonist.

27b. A composition, method, use, or kit according to paragraph 26b, wherein the PD1 antagonist is selected from pembrolizumab, nivolumab, MEDI0680, PDR001 (spartalizumab), Camrelizumab, AUNP12, Pidilizumab Cemiplimab (REGN-2810), AMP-224, BGB-A317 (Tisleizumab), and BGB-108.

28b. A composition, method, use, or kit according to any one of paragraphs 1b to 23b, wherein the secondary agent is a PD-L1 antagonist.

29b. A composition, method, use, or kit according to paragraph 28b, wherein the PD-L1 antagonist is selected from atezolizumab (Tecentriq), BMS-936559/MDX-1105, durvalumab/MEDI4736, and MSB0010718C (Avelumab).

30b. A composition, method, use, or kit according to any one of paragraphs 1b to 23b, wherein the secondary agent is a GITR (Glucocorticoid-Induced TNFR-Related protein) agonist.

31b. A composition, method, use, or kit according to paragraph 30b, wherein the GITR (Glucocorticoid-Induced TNFR-Related protein) agonist is selected from MEDl1873, TRX518, GWN323, MK-1248, MK4166, BMS-986156 and INCAGN1876.

32b. A composition, method, use, or kit according to any one of paragraphs 1b to 23b, wherein the secondary agent is a OX40 agonist.

33b. A composition, method, use, or kit according to paragraph 32b, wherein the OX40 agonist is selected from MEDl0562, MEDl6383, MOXR0916, RG7888, OX40mAb24, INCAGN1949, GSK3174998, and PF-04518600.

34b. A composition, method, use, or kit according to any one of paragraphs 1b to 23b, wherein the secondary agent is a CTLA-4 antagonist.

35b. A composition, method, use, or kit according to paragraph 34b, wherein the CTLA-4 antagonist is selected from ipilimumab and Tremelimumab.

36b. A composition, method, use, or kit according to any one of paragraphs 1b to 23b, wherein the secondary agent is Cytarabine.

37b. A composition, method, use, or kit according to any one of paragraphs 1b to 23b, wherein the secondary agent is Fludarabine.

38b. A composition, method, use, or kit according to any one of paragraphs 1b to 23b, wherein the secondary agent is a hypomethylating agent.

39b. A composition, method, use, or kit according to paragraph 38b, wherein the hypomethylating agent is selected from 5-azacytidine (azacitidine) and 5-aza-2’-deoxycytidine (decitabine).

40b. A composition, method, use, or kit according to any one of paragraphs 1b to 23b, wherein the secondary agent is an agent that upregulates HER2 expression.

41b. A composition, method, use, or kit according to paragraph 40b, wherein the agent that upregulates HER2 expression is selected from gemcitabine and tamoxifen.

42b. A composition, method, use, or kit according to any preceding paragraph, wherein the anti-CD20 agent is rituximab.

43b. A composition, method, use, or kit according to any preceding paragraph, wherein the anti-CD20 agent is selected from obinutuzumab, Ibritumomab tiuxetan, tositumomab, Ofatumumab, Ocaratuzumab, Ocrelizumab, and Veltuzumab.

### STATEMENTS OF INVENTION

1. A method of selecting an individual as suitable for treatment with a combination of an ADC and a secondary agent, wherein the individual is selected for treatment if the individual has been treated with an anti-CD20 agent.

2. A method of selecting an individual as suitable for treatment with a combination of an ADC and a secondary agent, wherein the individual is selected for treatment if the individual is being treated with an anti-CD20 agent.

3. The method according to any one of the preceding paragraphs, wherein the individual is selected for treatment if the individual is refractory to treatment, or further treatment, with the anti-CD20 agent.

4. A method for treating a disorder in an individual, the method comprising:
(i) selecting an individual as suitable for treatment by a method according to any one of paragraphs 1 to 3; and
(ii) administering to the individual an effective amount of the combination of an ADC and a secondary agent.

5. The method according to paragraph 4, further comprising administering an anti-CD20 agent in combination with the combination of an ADC and a secondary agent.

6. A method for treating a disorder in an individual, the method comprising administering to the individual an effective amount of an ADC, a secondary agent, and an anti-CD20 agent.

6. A method for treating a disorder in an individual, the method comprising administering to the individual an effective amount of an ADC, a secondary agent, and an anti-CD20 agent.

8. The method according to any one of paragraphs 5 to 7, wherein the treatment comprises administering the ADC and a secondary agent before the anti-CD20 agent, simultaneous with the anti-CD20 agent, or after the anti-CD20 agent.

9. The method according to any previous paragraph, wherein the treatment further comprises administering a chemotherapeutic agent.

10. The method according to any previous paragraph, wherein the individual is human.

11. The method according to any preceding paragraph, wherein the individual has a disorder or has been determined to have a disorder.

12. The method according to paragraph 11, wherein the individual has, or has been has been determined to have, a cancer which expresses CD19 or CD19+ tumour-associated non-tumour cells, such as CD19+ infiltrating cells.

13. The method according to any previous paragraph, wherein the individual is undergoing treatment with an anti-CD20 agent.

14. The method according to any previous paragraph, wherein the individual has undergone treatment with an anti-CD20 agent.

15. The method according to any previous paragraph, wherein the individual is refractory to treatment, or further treatment, with the anti-CD20 agent.

16. The method according to any one of the preceding paragraphs, wherein the treatment has increased efficacy as compared to monotherapy with either the ADC or anti-CD20 agent alone.

17. The method according to any preceding paragraph, wherein the ADC is an anti-CD19 ADC.

18. The method according to paragraph 17, wherein the anti-CD19 ADC is ADCx19.

19. The method according to any one of paragraph 1 to 16, wherein the ADC is an anti-CD22 ADC.

20. The method according to paragraph 19, wherein the anti-CD22 ADC is ADCx22.

21. The method according to any previous paragraph, wherein the disorder is a proliferative disease.

22. The method of paragraph 21, wherein the disorder is cancer.

23. The method of paragraph 22, wherein the disorder is selected from the group comprising: non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL).

24. The method of paragraph 22, wherein the disorder is characterized by the presence of one or more solid tumours.

25. The method of paragraph 24, wherein the solid tumour is pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, or head and neck cancer.

26. The method according to any previous paragraph, wherein the anti-CD20 agent is selected from the group consisting of: rituximab, obinutuzumab, Ibritumomab tiuxetan, tositumomab, Ofatumumab, Ocaratuzumab, Ocrelizumab, and Veltuzumab.

27. The method according to any one of paragraphs 1 to 25, wherein the anti-CD20 agent is rituximab.

28. An ADC for use in a method of treatment according to any one of paragraphs 4 to 24.

29. A composition comprising an ADC, for use in a method of treatment according to any one of paragraphs 4 to 27.

30. a secondary agent for use in a method of treatment according to any one of paragraphs 4 to 27.

31. A composition comprising a secondary agent, for use in a method of treatment according to any one of paragraphs 4 to 27.

32. An anti-CD20 agent for use in a method of treatment according to any one of paragraphs 5 to 27.

33. A composition comprising an anti-CD20 agent, for use in a method of treatment according to any one of paragraphs 5 to 27.

34. Use of an ADC in the manufacture of a medicament for treating a disorder in an individual, wherein the treatment comprises the method of any one of paragraphs 4 to 27.

35. Use of A secondary agent in the manufacture of a medicament for treating a disorder in an individual, wherein the treatment comprises the method of any one of paragraphs 4 to 27.

36. Use of an anti-CD20 agent in the manufacture of a medicament for treating a disorder in an individual, wherein the treatment comprises the method of any one of paragraphs 5 to 27.

37. A kit comprising:
a first medicament comprising an ADC;
a package insert comprising instructions for administration of the first medicament according to the method of any one or paragraphs 4 to 27.

38. The kit according to paragraph 37, further comprising: A second medicament comprising an anti-CD20 agent.

39. A composition, method, use, or kit according to any one of the preceding paragraphs, wherein the secondary agent is a Bruton’s Tyrosine Kinase inhibitor (BTKi).

40. A composition, method, use, or kit according to paragraph 39, wherein the Bruton’s Tyrosine Kinase inhibitor (BTKi) is selected from Ibrutinib (Imbruvica), Acalabrutinib/ACP-196, ONO/GS-4059, Spebrutinib/AVL-292/CC-292, HM71224 (Poseltinib) and BGB-3111 (Zanubrutinib).

41. A composition, method, use, or kit according to any one of paragraphs 1 to 38, wherein the secondary agent is a PD1 antagonist.

42. A composition, method, use, or kit according to paragraph 41, wherein the PD1 antagonist is selected from pembrolizumab, nivolumab, MEDI0680, PDR001 (spartalizumab), Camrelizumab, AUNP12, Pidilizumab Cemiplimab (REGN-2810), AMP-224, BGB-A317 (Tisleizumab), and BGB-108.

43. A composition, method, use, or kit according to any one of paragraphs 1 to 38, wherein the secondary agent is a PD-L1 antagonist.

44. A composition, method, use, or kit according to paragraph 43, wherein the PD-L1 antagonist is selected from atezolizumab (Tecentriq), BMS-936559/MDX-1105, durvalumab/MEDI4736, and MSB0010718C (Avelumab).

45. A composition, method, use, or kit according to any one of paragraphs 1 to 38, wherein the secondary agent is a GITR (Glucocorticoid-Induced TNFR-Related protein) agonist.

46. A composition, method, use, or kit according to paragraph 45, wherein the GITR (Glucocorticoid-Induced TNFR-Related protein) agonist is selected from MEDl1873, TRX518, GWN323, MK-1248, MK4166, BMS-986156 and INCAGN1876.

48. A composition, method, use, or kit according to paragraph 47, wherein the OX40 agonist is selected from MEDl0562, MEDl6383, MOXR0916, RG7888, OX40mAb24, INCAGN1949, GSK3174998, and PF-04518600.

49. A composition, method, use, or kit according to any one of paragraphs 1 to 38, wherein the secondary agent is a CTLA-4 antagonist.

50. A composition, method, use, or kit according to paragraph 49, wherein the CTLA-4 antagonist is selected from ipilimumab and Tremelimumab.

51. A composition, method, use, or kit according to any one of paragraphs 1 to 38, wherein the secondary agent is Cytarabine.

52. A composition, method, use, or kit according to any one of paragraphs 1 to 38, wherein the secondary agent is Fludarabine.

53. A composition, method, use, or kit according to any one of paragraphs 1 to 38, wherein the secondary agent is a hypomethylating agent.

54. A composition, method, use, or kit according to paragraph 53, wherein the hypomethylating agent is selected from 5-azacytidine (azacitidine) and 5-aza-2’-deoxycytidine (decitabine).

55. A composition, method, use, or kit according to any one of paragraphs 1 to 38, wherein the secondary agent is an agent that upregulates HER2 expression.

56. A composition, method, use, or kit according to paragraph 55, wherein the agent that upregulates HER2 expression is selected from gemcitabine and tamoxifen.

### EXAMPLES

In the following examples:
the FTP is preferably CD19 or CD22.
Cell lines expressing CD19 suitable for use in the examples include Ramos, Daudi, Raji, WSU-DLCL and NALM-6 cells.
Cell lines expressing CD22 suitable for use in the examples include Ramos, Daudi, Raji, WSU-DLCL and NALM-6 cells.
Disease A - Diffuse Large B-cell Lymphoma/DLBC is an aggressive type of non-Hodgkin lymphoma that develops from the B-cells in the lymphatic system. It constitutes the largest subgroup of non-Hodgkin lymphoma.
Disease B - Mantle Cell Lymphoma/MCL is a rare B-cell NHL that most often affects men over the age of 60. The disease may be aggressive (fast growing) but it can also behave in a more indolent (slow growing) fashion in some patients. MCL comprises about five percent of all NHLs.
Disease C - Follicular lymphoma/FL is a fairly indolent type of NHL with long survival time but for which it is very difficult to achieve a cure; it can also transform into more aggressive forms of lymphoma.

### Example 1

To show that a PBD-ADC can induce ICD and therefore can be a suitable combination agent with immune-oncology (IO) drugs, cell lines expressing a first target protein (FTP), will be incubated for 0, 6, 24 and 48 hours with etoposide (negative control) and oxaliplatin (positive control), 1 µg/mL ADC, 1 µg/mL anti-FTP (the antibody in ADC) and 1 µg/mL of B12-SG3249 (a non-binding control ADC with the same PBD payload as ADC).

After Incubation, the amount of AnnexinV-/PI+ (early apoptotic cells) will be measured by Flow cytometry together with the upregulation of surface calreticulin and HSP-70. ER stress will be measured by Northern blot analyses of IRE1 phosphorylation, ATF4 and JNK phosphorylation.

### Example 2

In a separate experiment, cell lines expressing FTPs will be incubated for 0, 6, 24 and 48 hours with etoposide (negative control) and oxaliplatin (positive control), 1 µg/mL ADC (ADC targeting FTP with a PBD dimer warhead), 1 µg/mL anti-FTP (the antibody in ADC) and 1 µg/mL of B12-SG3249 (a non-binding control ADC with the same PBD payload as ADC).

After incubation, the cells are washed, and fed to human Dendritic cells (DCs) for an additional 24 h. Activation of the DCs is subsequently measured by increased surface expression of CD86 on the DC population (as determined by Flow cytometry) and by measuring DC mediated release of IL-8 and MIP2.

### Example 3

The purpose of this study is to preliminarily assess the safety, tolerability, pharmacological and clinical activity of this combination

The following cancer types have been chosen for study: Disease A, Disease B, and Disease C

Evidence for efficacy as single agents exists for both drugs:
ADC (see, for example, WO2014/057117, WO2016/166298, WO2014/057122, and WO2016/166307)
Secondary agent (see KS Peggs et al.2009, Clinical and Experimental Immunology, 157: 9-19 [doi:10.1111/j.1365-2249.2009.03912.x])

This primary purpose of this study is to explore whether these agents can be safely combined, and if so, will identify the doses(s) and regimens appropriate for further study. The study will also assess whether each combination induces pharmacologic changes in tumor that would suggest potential clinical benefit.

In addition, it will provide preliminary evidence that a combination may increase the response rate and durability of response compared with published data for treatment with single agent ADC or secondary agent.

Each disease group may include a subset of patients previously treated with the secondary agent to explore whether combination therapy might overcome resistance to secondary agent therapy. For each disease, it is not intended to apply specific molecular selection as the data available at present generally do not support excluding patients on the basis of approved molecular diagnostic tests.

### Rationale for ADC Starting Dose

The RDE for already established for ADC (in ug/kg administered every three weeks) will be used for all patients in this study. To ensure patient safety, a starting dose below the RDE will be used; the starting dose level will be one where patient benefit could still be demonstrated in study ADC1, suggesting that patients enrolled at such dose level will gain at least some benefit by taking part.

### Rationale for Secondary Agent Starting Dose

The RDE for already established for the secondary agent (in ug/kg administered every three weeks) will be used for all patients in this study. To ensure patient safety, a starting dose below the RDE will be used; the starting dose level will be one where patient benefit could still be demonstrated in study SA1, suggesting that patients enrolled at such dose level will gain at least some benefit by taking part.

Objectives and related endpoints

| | |
|---|---|
| Objective | Endpoint |
| Primary Objective | Frequency and severity of treatment-emergent AEs and SAEs |
| To characterize the safety and tolerability of ADC in combination with the secondary agent, and to identify the recommended dose and schedules for future studies | Changes between baseline and post-baseline laboratory parameters and vital signs |
| | Incidence of dose limiting toxicities (DLTs) during the first cycle of treatment (dose escalation only) |
| | Frequency of dose interruptions and dose reductions |
| Secondary Objectives | |
| To evaluate the clinical activity of the combination of ADC with the secondary agent | ORR, DOR, PFS, OS |
| To characterize the pharmacokinetic (PK) profile of each of the two compounds ADC and the secondary agent | AUC and Cmax for each compound |
| Evidence for immunogenicity and ADAs to ADC | Anti-Drug-Antibodies (ADAs) before, during and after treatment with ADC |
| Exploratory Objectives | |
| To examine potential correlation of PK profiles with safety/tolerability and efficacy | Correlation coefficients between AUC and/or Cmax of each compound or a compound measure and any of the safety or efficacy variables |
| To characterize changes in the immune infiltrate in tumors | Immunohistochemistry of pre- and on-treatment tumor biopsies, |
| To characterize changes in circulating levels of cytokines in plasma and markers of activation in circulating immune cells | Measurements (e.g. via ELISA) of immunologically relevant cytokines in plasma or serum; staining levels for activation markers of circulating immune cells (e.g. FACS) |

### Study Design

This phase 1b, multi-center, open-label study to characterize the safety, tolerability, pharmacokinetics (PK), pharmacodynamics (PD) and antitumor activity of the ADC in combination with the secondary agent, in patients with disease A, disease B, and disease C.

The study is comprised of a dose escalation part followed by a dose expansion part.

Dose escalation will start with reduced starting doses (compared to their respective recommended phase 2 or licensed dose levels), for both ADC and the secondary agent, to guarantee patient safety. Starting doses will be 33% (or 50%) of the RDE for each compound. Subsequently, doses will be first escalated for the secondary agent until the RDE or licensed dose has been reached, or a lower dose if necessary for tolerability reasons. Then, the dose for ADCwill be escalated, until the RDE for combination treatment is reached. This is visualized in the below diagram:

| | |
|---|---|
| [image] | A perceived safe starting dose of 33% of the intended efficacious dose is proposed for both compounds, but this may need adaptation to lower or higher, as the individual risk profile for the combination may be. |
| | Compound 1 should be the compound for which an efficatious clinical dose has been firmly established (at 100%), and which is therefore aimed to be reached quickly in the trial patients by first escalating the dose of this compound. |

If the dose combination is determined to be safe, it may be tested in additional patients to confirm the safety and tolerability at that dose level. Further tailoring of the dose of each compound may be conducted, and/or the regimen may be modified.

The dose escalation of the combination will be guided by a Bayesian Logistic Regression Model (BLRM) based on any Dose Limiting Toxicities (DLTs) observed in the first (or first two, TBC) cycles of therapy. Use of a BLRM is a well-established method to estimate the maximum tolerated dose (MTD)/ recommended dose for expansion (RDE) in cancer patients. The adaptive BLRM will be guided by the Escalation With Overdose Control (EWOC) principle to control the risk of DLT in future patients on the study. The use of Bayesian response adaptive models for small datasets has been accepted by FDA and EMEA (“Guideline on clinical trials in small populations”, Feb. 1, 2007) and endorsed by numerous publications (Babb et al. 1998, Neuenschwander et al. 2008).

The decisions on new dose combinations are made by the Investigators and sponsor study personnel in a dose escalation safety call (DESC) based upon the review of patient tolerability and safety information (including the BLRM summaries of DLT risk, if applicable) along with PK, PD and preliminary activity information available at the time of the decision.

Once the MTD(s)/RDE is determined for the combination, the expansion part of the study may be initiated to further assess the safety, tolerability and preliminary efficacy. For combinations with IO, changes in the immune infiltrate in tumors will also be characterized following combination treatment in the target disease indications.

Given the available prior clinical experience with the agents in this study, it is expected that in most cases a combination dose can be identified without testing a large number of dose levels or schedules. To assess the pharmacodynamic activity of the combinations, patients will be asked to undergo a tumor biopsy at baseline and again after approximately two cycles of therapy. For IO combo: The extent of the change in tumor infiltration by immune cells including lymphocytes and macrophages will contribute to a decision on any potential benefit.

### Dose Escalation Part

During the dose escalation part of the study, patients will be treated with a fixed dose of ADC administered i.v., and increasing doses of the secondary agent until the RDE for the secondary agent has been reached. Subsequently, doses of ADC are increased (in different cohorts) while the dose for the secondary agent is kept constant.

Two to approximately 3 or 4 patients with disease A, disease B or disease C will be treated in each escalation cohort until the determination of MTD(s)/RDE(s) is determined.

There will be a 24-hour observation before enrolling the second patient at Dose Level 1. The DLT observation period at each dose level is either 1 cycle (3 weeks) or 2 cycles (6 weeks) as mandated by the appropriate authorities for IO therapies, after which it will be determined whether to escalate to the next dose level, stay at the current dose level, or de-escalate to the previous dose level for the next cohort. There will be no de-escalation from Dose Level 1. Intrapatient dose escalation is not permitted.

Dose escalation is not permitted unless 2 or more patients have complete DLT information through the first cycle in any given dose level. Dose escalation will be determined by using a mCRM with a target DLT rate of 30% and an equivalence interval of 20% to 35%, and with dose escalation-with-overdose-control (EWOC) and no dose skipping.

Patients will be assigned to a cohort that is actively enrolling. Dose escalation will be performed in each combination following the completion of one cycle of treatment. Safety assessments including adverse events (AEs) and laboratory values will be closely monitored for all enrolled patients in order to identify any DLTs. A single MTD/RDE will be defined; a disease-specific MTD/RDE will not be established.

The mCRM will be implemented for DE under the oversight of a Dose Escalation Steering Committee (DESC). The DESC will confirm each escalating dose level after reviewing all available safety data. PK data from patients in that dose level and prior dose levels may also inform decision making. The DESC may halt dose escalation prior to determining the MTD based on emerging PK, PD, toxicity or response data.

Additional patients may be included at any dose level to further assess the safety and tolerability if at least 1 patient in the study has achieved a partial response or better, or if further evaluation of PK or PD data is deemed necessary by the DESC to determine the RDE.

Dose Escalation will be stopped after 3 cohorts (or at least 6 patients) are consecutively assigned to the same dose level. If the MTD is not reached, the recommended dose for expansion (RDE) will be determined. Prior to the determination of the MTD/RDE a minimum of 6 patients must have been treated with the combination.

It is intended that paired tumor biopsies will be obtained from patients during dose escalation. Analysis of these biopsies will contribute to a better understanding of the relationship between the dose and the pharmacodynamic activity of the combination.

### Safety Oversight by the Dose Escalation Steering Committee

A DESC comprised of ADC Therapeutics and the investigators will review patient safety on an ongoing basis during the DE to determine if the dose escalation schedule prescribed by the mCRM warrants modification. In addition to safety observations, PK and/or PD data may also inform decision making. Intermediate doses may be assigned after agreement between ADC Therapeutics and investigators. The DESC may continue to provide oversight during Part 2. No formal Data Safety Monitoring Board (DSMB) will be used.

### Dose Expansion Part

Once the MTD/RDE has been declared, dose expansion part may begin. The main objective of the expansion part is to further assess the safety and tolerability of the study treatment at the MTD/RDE and to gain a preliminary understanding of the efficacy of the combination compared to historical single agent efficacy data.

An important exploratory objective is to assess changes in the immune infiltrate in tumor in response to treatment. This will be assessed in paired tumor biopsies collected from patients, with a minimum of ten evaluable biopsy pairs (biopsy specimens must contain sufficient tumor for analysis) in patients treated at the MTD/RDE. If this is not feasible, collection of these biopsies may be stopped. A minimum of 10 to 20 patients are planned to be treated in each investigational arm,

Several different investigational arms will open, one per disease. A total of nine investigational arms may be run in the dose expansion. Should enrollment for any of these groups not be feasible, then enrollment to that group may be closed before the 10 to 20 patients target is met.

In each treatment group a maximum of approximately six patients who have received and progressed on prior single administration (i.e. not in combination) secondary agent therapy will be allowed to be treated. This number may be increased if a combination shows promise of overcoming resistance to prior treatment with single administration secondary agent.

### Patient Population

The study will be conducted in adult patients with advanced Disease A, Disease B or Disease C as outlined above. The investigator or designee must ensure that only patients who meet all the following inclusion and none of the exclusion criteria are offered treatment in the study.

### Inclusion Criteria

Patients eligible for inclusion in this study have to meet all of the following criteria:
1. Written informed consent must be obtained prior to any procedures
2. Age 18 years.
3. Patients with advanced/metastatic cancer, with measurable disease as determined by RECIST version 1.1, who have progressed despite standard therapy or are intolerant to standard therapy, or for whom no standard therapy exists. Patients must fit into one of the following groups:
   Disease A
   Disease B
   Disease C
4. ECOG Performance Status 0-1 (or 2 TBC
5. TBC: Patient must have a site of disease amenable to biopsy, and be a candidate for tumor biopsy according to the treating institution’s guidelines. Patient must be willing to undergo a new tumor biopsy at baseline, and again during therapy on this study.
6. Prior therapy with the secondary agent or related compounds (i.e. same MOA) is allowed

### Exclusion Criteria

Patients eligible for this study must not meet any of the following criteria:
1. History of severe hypersensitivity reactions to other mAbs (OR to same backbone mAb as in ADC OR to same IO mAb if applicable)
2. Known history of positive serum human ADA to backbone of mAb as in ADC
3. Central Nervous System (CNS) disease only (if applicable)
4. Symptomatic CNS metastases or evidence of leptomeningeal disease (brain MRI or previously documented cerebrospinal fluid (CSF) cytology)
   Previously treated asymptomatic CNS metastases are permitted provided that the last treatment (systemic anticancer therapy and-or local radiotherapy) was completed >= 8 weeks prior to 1^{st} day of dosing, except usage of low dose steroids on a taper is allowed)
   Patients with discrete dural metastases are eligible.
5. Patient having out of range laboratory values defined as:
   Serum creatinine <= 1.5 x ULN. If serum creatinine > 1.5, the creatinineclearance (calculated using Cockcroft-Gault formula, or measured) must be > 60 mL/min/1.73 m2 for a patient to be eligible
   Total bilirubin > 1.5 x ULN, except for patients with Gilbert’s syndrome who are excluded if total bilirubin > 3.0 x ULN or direct bilirubin > 1.5 x ULN
   Alanine aminotransferase (ALT) > 3 x ULN, except for patients that have tumor involvement of the liver, who are excluded if ALT > 5 x ULN
   Aspartate aminotransferase (AST) > 3 x ULN, except for patients that have tumor involvement of the liver, who are excluded if AST > 5 x ULN
   Absolute neutrophil count< 1.0 x 10e9 /L
   Platelet count< 75 x 10e9 /L
   Hemoglobin (Hgb) < 8 g/dL
   Potassium, magnesium, calcium or phosphate abnormality > CTCAE grade 1 despite appropriate replacement therapy
6. Impaired cardiac function or clinically significant cardiac disease, including any of the following:
   Clinically significant and/or uncontrolled heart disease such as congestive heart failure requiring treatment (NYHA grade III or IV) or uncontrolled hypertension defined by a Systolic Blood Pressure (SBP) 160 mm Hg and/or Diastolic Blood Pressure (DBP) 100 mm Hg, with or without anti-hypertensive medication.
   QTcF >470 msec for females or >450 msec for males on screening ECG using Fridericia’s correction, congenital long QT syndrome
   Acute myocardial infarction or unstable angina pectoris < 3 months (months prior to study entry
   Clinically significant valvualr disease with documented compromise in cardiac function
   Symptomatic pericarditis
   History of or ongoing documented cardiomyopathy
   Left Ventricular Ejection Fraction (LVEF) <40%, as determined by echocardiogram (ECHO) or Multi gated acquisition (MUGA) scan
   History or presence of any clinically significant cardiac arrhythmias, e.g. ventricular, supraventricular, nodal arrhythmias, or conduction abnormality (TBC qualifier: ... requiring a pacemaker or not controlled with medication)
   Presence of unstable atrial fibrillation (ventricular response rate> 100 bpm). NOTE: Patients with stable atrial fibrillation can be enrolled provided they do not meet other cardiac exclusion criteria.
   Complete left bundle branch block (LBBB), bifascicular block
   Any clinically significant ST segment and/or T-wave abnormalities
7. Toxicity attributed to prior IO therapy that led to discontinuation of therapy. Adequately treated patients for drug-related skin rash or with replacement therapy for endocrinopathies are not excluded, provided these toxicities did not lead to the discontinuation of prior treatment.
8. Patients with active, known or suspected autoimmune disease. Subjects with vitiligo, type I diabetes mellitus, residual hypothyroidism due to autoimmune condition only requiring hormone replacement, psoriasis not requiring systemic treatment, or conditions not expected to recur in the absence of an external trigger are permitted to enroll, provided the trigger can be avoided.
9. Human Immunodeficiency Virus (HIV), or active Hepatitis B (HBV) or Hepatitis C (HCV) virus infection
   Testing is not mandatory to be eligible. Testing for HCV should be considered if the patient is at risk for having undiagnosed HCV (e.g. history of injection drug use).
10. Malignant disease, other than that being treated in this study. Exceptions to this exclusion include the following: malignancies that were treated curatively and have not recurred within 2 years prior to study treatment; completely resected basal cell and squamous cell skin cancers; any malignancy considered to be indolent and that has never required therapy; and completely resected carcinoma in situ of any type.
11. Systemic anti-cancer therapy within 2 weeks of the first dose of study treatment. For cytotoxic agents that have major delayed toxicity, e.g. mitomycin C and nitrosoureas, 4 weeks is indicated as washout period. For patients receiving anticancer immunotherapies such as CTLA-4 antagonists, 6 weeks is indicated as the washout period.
12. Active diarrhea CTCAE grade 2 or a medical condition associated with chronic diarrhea (such as irritable bowel syndrome, inflammatory bowel disease)
13. Presence of 2: CTCAE grade 2 toxicity (except alopecia, peripheral neuropathy and ototoxicity, which are excluded if >= CTCAE grade 3) due to prior cancer therapy.
14. Active infection requiring systemic antibiotic therapy.
15. Active ulceration of the upper GI tract or GI bleeding
16. Active bleeding diathesis or on oral anti-vitamin K medication (except low-dose warfarin and aspirin or equivalent, as long as the INR <= 2.0)
17. Active autoimmune disease, motor neuropathy considered of autoimmune origin, and other CNS autoimmune disease
18. Patients requiring concomitant immunosuppressive agents or chronic treatment with corticoids except:
   replacement dose steroids in the setting of adrenal insufficiency
   topical, inhaled, nasal and ophthalmic steroids are allowed
19. Use of any live vaccines against infectious diseases (e.g. influenza, varicella, pneumococcus) within 4 weeks of initiation of study treatment (NB the use of live vaccines is not allowed through the whole duration of the study)
20. Use of hematopoietic colony-stimulating growth factors (e.g. G-CSF, GMCSF, M-CSF) < 2 weeks prior start of study drug. An erythroid stimulating agent is allowed as long as it was initiated at least 2 weeks prior to the first dose of study treatment.
21. Major surgery within 2 weeks of the first dose of study treatment (NB mediastinoscopy, insertion of a central venous access device, or insertion of a feeding tube are not considered major surgery).
22. Radiotherapy within 2 weeks of the first dose of study drug, except for palliative radiotherapy to a limited field, such as for the treatment of bone pain or a focally painful tun1 or mass. To allow for assessment of response to treatment, patients must have remaining measurable disease that has not been irradiated
23. Participation in an interventional, investigational study within 2 weeks of the first dose of study treatment.
24. Any medical condition that would, in the investigator’s judgment, prevent the patient’s participation in the clinical study due to safety concerns, compliance with clinical study procedures or interpretation of study results.
25. Sexually active males unless they use a condom during intercourse while taking drug and for 90 days after stopping study treatment and should not father a child in this period. A condom is required to be used also by vasectomized men in order to prevent delivery of the drug via seminal fluid.
26. Pregnant or lactating women, where pregnancy is defined as the state of a female after conception and until the termination of gestation, confirmed by a positive hCG laboratory test. In rare cases of an endocrine-secreting tumor, hCG levels may be above normal limits but wi th no pregnancy in the patient. In these cases, there should be a repeat serum hCG test (with a non-rising result) and a vaginal/pelvic ultrasound to rule out pregnancy. Upon confirmation of results and discussion with the Medical representative, these patients may enter the study.
27. Women of child-bearing potential, defined as all women physiologically capable of becoming pregnant, unless they are using highly effective methods of contraception during study treatment and for 90 days after the last any dose of study treatment.

Highly effective contraception methods include:
Total abstinence (when this is in line with the preferred and usual lifestyle of the patient. Periodic abstinence (e.g., calendar, ovulation, symptothermal, post-ovulation methods) and withdrawal are not acceptable methods of contraception
Female sterilization (have had surgical bilateral oophorectomy with or without hysterectomy), total hysterectomy or tubal ligation at least 6 weeks before taking study treatment. In case of oophorectomy alone, only when the reproductive status of the woman has been confirmed by follow up hormone level assessment
Male sterilization (at least 6 months prior to screening). For female patients on the study the vasectomized male partner should be the sole partner for that patient.
Use of oral (estrogen and progesterone), injected or implanted combined hormonal methods of contraception or placement of an intrauterine device (IUD) or intrauterine system (IUS) or other forms of hormonal contraception that have comparable efficacy (failure rate <1%), for example hormone vaginal ring or transdermal hormone contraception.
   In case of use of oral contraception, women should have been stable on the same pill for a minimum of 3 months before taking study treatment.
   Women are considered post-menopausal and not of child bearing potential if they have had 12 months of natural (spontaneous) amenorrhea with an appropriate clinical profile (e.g. age appropriate, history of vasomotor symptoms) or have had surgical bilateral oophorectomy (with or without hysterectomy) or tubal ligation at least 6 weeks ago. In the case of oophorectomy alone, only when the reproductive status of the woman has been confirmed by follow up hormone level assessment is she considered not of child bearing potential.

### Dose-Limiting Toxicities and Dose Modification Guidelines

A dose-limiting toxicity (DLT) is defined as any of the following events thought to be at least possibly related to ADC per investigator judgment that occurs during the 21-day DLT evaluation period. Toxicity that is clearly and directly related to the primary disease or to another etiology is excluded from this definition.

### DLT Defintions

A hematologic DLT is defined as:
Grade 3 or 4 febrile neutropenia or neutropenic infection
Grade 4 neutropenia lasting >7 days
Grade 4 thrombocytopenia
Grade 3 thrombocytopenia with clinically significant bleeding, or Grade 3 thrombocytopenia requiring a platelet transfusion
Grade 3 anemia that requires transfusion
Grade 4 anemia

A non-hematologic DLT is defined as:
Grade 4 non-hematologic toxicity
Grade 3 non-hematologic toxicity lasting >3 days despite optimal supportive care or medical intervention
A case of Hy’s law (AST and/or ALT > 3x ULN and bilirubin > 2x ULN, and without initial findings of cholestasis (serum alkaline phosphatase (ALP) activity < 2x ULN) and no other reason that could explain the combination of increased transaminases and serum total bilirubin, such as viral hepatitis A, B, or C, preexisting or acute liver disease, or another drug capable of causing the observed injury)
Grade 3 or higher hypersensitivity/infusion-related reaction (regardless of premedication). A grade 3 hypersensitivity / infusion-related reaction that resolves within 8 hours after onset with appropriate clinical management does not qualify as a DLT.
LVEF decrease to < 40% or >20% decrease from baseline
Grade 4 tumor lysis syndrome (Grade 3 TLS will not constitute DLT unless it leads to irreversible end-organ damage)

The following conditions are not considered non-hematologic DLT:
Grade 3 fatigue for ≤ 7 days
Grade 3 diarrhea, nausea, or vomiting in the absence of premedication that responds to therapy and improves by at least 1 grade within 3 days for Grade 3 events or to ≤

Grade 1 within 7 days.
AST or ALT elevation ≥ 5 x ULN but ≤ 8 x ULN, without concurrent elevation in bilirubin, that downgrades to ≤ Grade 2 within 5 days after onset.
Grade 3 serum lipase or serum amylase for ≤ 7 days if without clinical signs or symptoms of pancreatitis

Patients who experience a DLT that resolves or stabilizes with appropriate medical management may continue treatment at the discretion of the investigator in consultation with the sponsor.

### Dose Modifications

Guidelines for management of specific toxicities are detailed in the table below. For management of events not specified in the tables, the following may serve as a guidance to investigators:

| AE Grade | ADC Management Guideline |
|---|---|
| 1 | No dose adjustment is required. |
| 2 | First occurrence: |
| | Consider holding one or both drugs until improvement to ≤ Grade 1 or baseline. Up to 1 dose of one or both drugs may be skipped to permit improvement. If improvement to ≤ Grade 1 or baseline occurs within 21 days from the last scheduled (but missed) dose of one or both drugs, continue one or both drugs at the original assigned dose level in subsequent treatment cycles. |
| | If improvement to ≤ Grade 1 or baseline does not occur within 21 days from the last scheduled (but missed) dose, permanently discontinue one or both drugs. |
| | Second occurrence: |
| | Hold one or both drugs until improvement to ≤ Grade 1 or baseline. Up to 1 dose of one or both drugs may be skipped to permit resolution. If improvement to ≤ Grade 1 or baseline occurs within 21 days from the last scheduled (but missed) dose, continue one or both drugs at 1 dose level below the original assigned dose in subsequent treatment cycles. If improvement to ≤ Grade 1 or baseline does not occur within 21 days from the last scheduled (but missed) dose, permanently discontinue one or both drugs. |
| | Third occurrence: |
| | Permanently discontinue one or both drugs. |
| 3 | First occurrence: |
| | Hold one or both drugs until improvement to _{≤} Grade 1 or baseline. Up to 1 dose of one or both drugs may be skipped to permit improvement, then continue at 1 dose level below the original assigned dose in subsequent treatment cycles. |
| | Second occurrence: |
| | Permanently discontinue one or both drugs |
| 4 | Permanently discontinue one or both drugs. |

### Example 4: In Vitro Synergy of ADCx19 & Rituximab

### Material & Methods

Ramos cells were cultured RPMI 1640 supplemented with 10% Hyclone FBS. The concentration and viability of cells from a sub-confluent (80-90% confluency) T75 flask were measured by trypan blue staining, and counted using the LUNA-II™ Automated Cell Counter. Cells were diluted to 2 x10⁵/ml, dispensed (50 µl/well) into 96-well flat-bottom plates. A chequerboard was set up in combining 10-fold dilutions of ADCTx19 or ADCTx22 and 10-fold dilutions of rituximab in RPMI before 50 µl of each dilution was transferred into the 96-well plate containing the cells. This plate was incubated at 37° C. in a CO2-gassed incubator for 4 days. At the end of the incubation period, cell viability was measured by MTS assay. MTS (Promega) was dispensed (20 µl per well) into each well and incubated for 4 hours at 37C in the CO2-gassed incubator. Well absorbance was measured at 490 nm. IC₅₀ was determined from the dose-response data using GraphPad Prism using the non-linear curve fit algorithm: sigmoidal dose-response curve with variable slope.

### Results

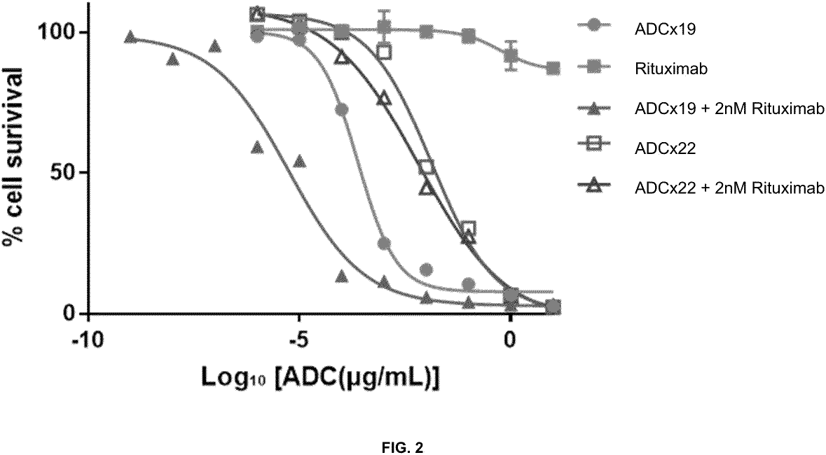

The results of the in vitro cytotoxicity assay are shown in the table below, and FIG. 2

| Added agents | EC50 |
|---|---|
| ADCx19 | 0.0002394 |
| Rituximab | 0.56 |
| ADCx19 + 2nM Rituximab | 0.000005472 |
| ADCx22 | 0.01331 |
| ADCx22 + 2nM Rituximab | 0.006576 |

### Discussion

When ADCx19 is combined with Rituximab, the potency is enhanced at least 10-fold, and the molecule becomes extremely potent. Rituximab by itself had no significant cytotoxic effect.

The same effect is not observed when Rituximab is administered together with ADCx22, despite the Ramos cells expressing significant amounts of both CD19 and CD22 antigens.

### Example 5: In Vitro Synergy of ADCx19 and Cytarabine

### Material & Methods

Cells were plated on day 1 at 10,000 cells/well in 96-well plates, three replicates per experiment and total n of 3. Combination drug was added on day 2 and incubated for 24 hours at 37℃, 5% CO₂.

On day 3 ADCx19 was added to cells containing drug, or media only as a control, in the dosage range 0.00004 pM - 50 nM at a 20 fold dilution and incubated for a further 4 days (3 x cell doubling time).

20 µl MTS was added to each well, and incubated for 2-3 hours under normal cell culture conditions. The OD was measured at 492 nm using a Thermo Labsystems Multiscan Ascent plate reader, and % growth calculated compared to the untreated control cells.

Growth curves were plotted using GraphPad Prism using the sigmoidal, 4PL, X is log (concentration) equation. IC50 values (dose of drug that inhibits growth by 50%) were determined. Percentage cell survival was converted into fraction affected (Fa) and the combination index (CI) for each dose calculated using CalcuSyn v2.11.

### Results

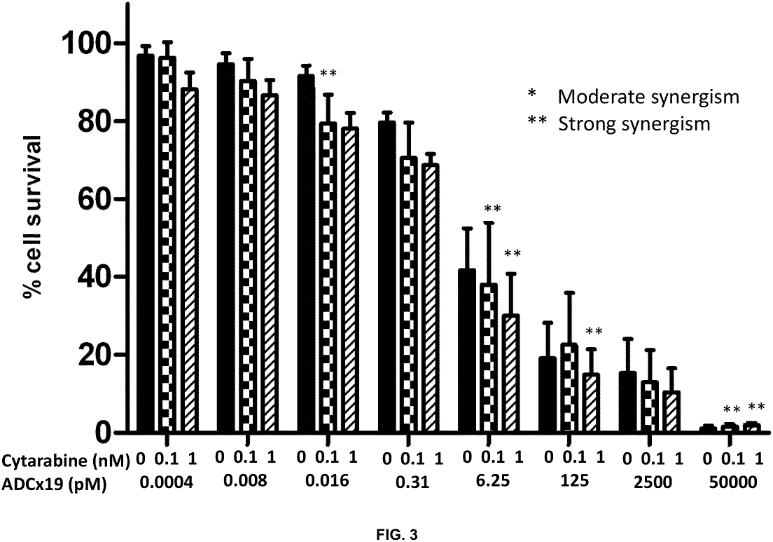

The results are shown in FIG. 3. As indicated in the figure legend, (*) indicates moderate synergism and (**) strong synergism as determined by CalcuSyn.

### Example 6: In Vitro Synergy of ADCx22 / Cytarabine and ADCx22 / Fludarabine

### Material & Methods

Cells were plated on day 1 at 10,000 cells/well in 96-well plates, three replicates per experiment and total n of 3. Combination drug (i.e. Cytarabine or Fludarabine) was added on day 2 and incubated for 24 hours at 37℃, 5% CO₂.

On day 3 ADCx22 was added to cells containing drug, or media only as a control, in the dosage range 0.005 pM - 50 nM at a 10 fold dilution and incubated for a further 4 days (3 x cell doubling time).

20 µl MTS was added to each well, and incubated for 2-3 hours under normal cell culture conditions. The OD was measured at 492 nm using a Thermo Labsystems Multiscan Ascent plate reader, and % growth calculated compared to the untreated control cells.

Growth curves were plotted using GraphPad Prism using the sigmoidal, 4PL, X is log (concentration) equation. IC50 values (dose of drug that inhibits growth by 50%) were determined. Percentage cell survival was converted into fraction affected (Fa) and the combination index (CI) for each dose calculated using CalcuSyn v2.11.

### Results

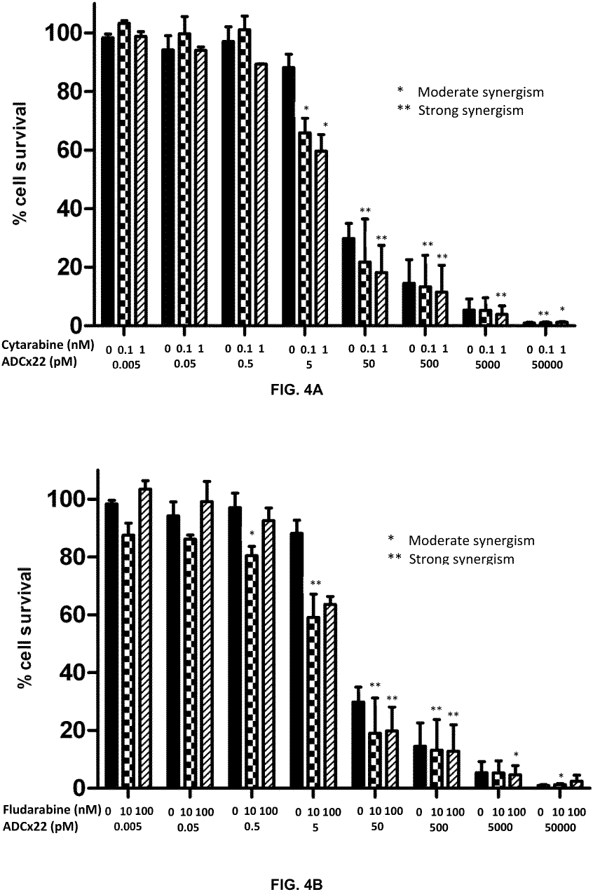

The results are shown in FIG. 4. As indicated in the figure legend, (*) indicates moderate synergism and (**) strong synergism as determined by CalcuSyn.

### Example 7: In Vivo Synergy of ADCx19 / Cytarabine and ADCx19 / Rituximab

### Material & Methods

Female severe combined immunodeficient mice (Fox Chase SCID®_{,} CB17/lcr-Prkdcscid/lcrlcoCrl, Charles River) were eight weeks old with a body weight (BW) range of 14.6 to 21.9 g on Day 1 of the study.

On the day of tumor implant, each test mouse received 1 x10⁷ WSU-DLCL2 tumor cells in 50% Matrigel implanted subcutaneously in the right flank. Tumor growth was monitored as the average size approached the target range of 100 to 150 mm³. Tumors were measured in two dimensions using calipers, and volume was calculated using the formula:

$$\text{Tumor} \text{Volume} \text{} \left({\text{mm}}^{\text{3}}\right) \text{=} {\text{w}}^{\text{2}} \times \text{l/2}$$

where w = width and I = length, in mm, of the tumor. Tumor weight may be estimated with the assumption that 1 mg is equivalent to 1 mm³ of tumor volume.

Fourteen days after tumor implantation, designated as Day 1 of the study, the animals were sorted into nine groups (n=8) with individual tumor volumes of 108 to 126 mm³ and group mean tumor volumes of 112.5 mm³.

On Day 1 of the study, ADCx19 was administered intravenously (i.v.) in a single injection (qd x 1) via tail vein injection; rituximab was administered i.v. once weekly for 4 weeks; cytarabine was administered intraperitoneally daily for 5 days. All doses were administered in a dosing volume of 10 mL/kg.

Tumors were measured using calipers twice per week, and each animal was euthanized when its tumor reached the endpoint volume of 1000 mm³ or at the end of the study, whichever came first. The study ended on Day 74.

### Results

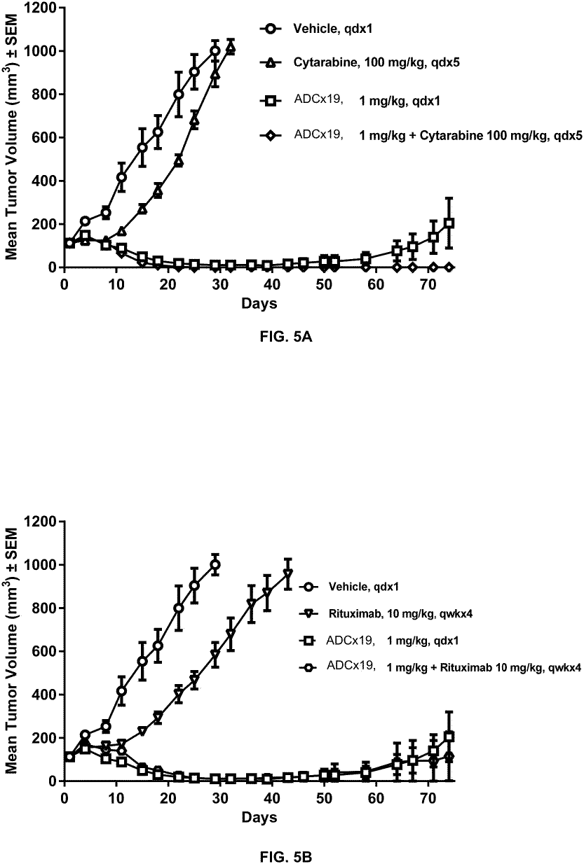

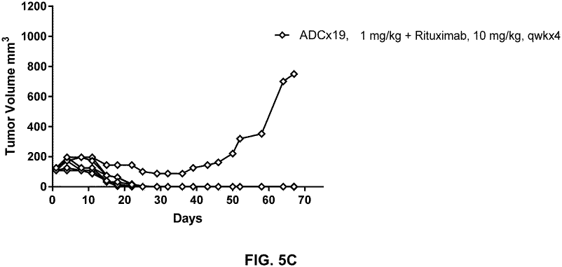

The results are shown in FIG. 5.

A single dose of ADCx19 at 1 mg/kg was selected based on historical data with the intention that this dose would be sub-optimal, and so allow for maximum assay sensitivity to synergy with the secondary agents. However, in this initial round of in vivo experiments, the 1 mg/kg dose of ADCx19 was more effective than anticipated, leaving reduced scope for recording synergy.

Notwithstanding this, the ADCx19/cytarabine data (FIG. 5A) is consistent with in vivo synergy. Similarly, the ADCx19/rituximab data (FIG. 5B) is consistent with in vivo synergy. [The apparent growth in the mean ADCx19/rituximab tumour size shown in FIG. 5B arises from the average including a single outlier where significant tumour growth was observed. This can be clearly seen in the single group data shown in FIG. 5C.]

### Example 8: In Vitro Synergy in CD19+ve Ramos Cell Line of ADCx19 With Each of Cytarabine, Fludarabine, Decitabine, and Gemcitabine

Cells were plated on day 1 at 10,000 cells/well in 96-well plates, three replicates per experiment and total n of 3. Combination drug was added on day 2 and incubated for 24 hours at 37℃, 5% CO₂.

On day 3 ADCx19 was added to cells containing drug, or media only as a control, in the dosage range 0.00004 pM - 50 nM at a 20 fold dilution and incubated for a further 4 days (3 x cell doubling time).

20 µl MTS was added to each well, and incubated for 2-3 hours under normal cell culture conditions. The OD was measured at 492 nm using a Thermo Labsystems Multiscan Ascent plate reader, and % growth calculated compared to the untreated control cells.

Growth curves were plotted using GraphPad Prism using the sigmoidal, 4PL, X is log (concentration) equation. IC50 values (dose of drug that inhibits growth by 50%) were determined. Percentage cell survival was converted into fraction affected (Fa) and the combination index (CI) for each dose calculated using CalcuSyn v2.11.

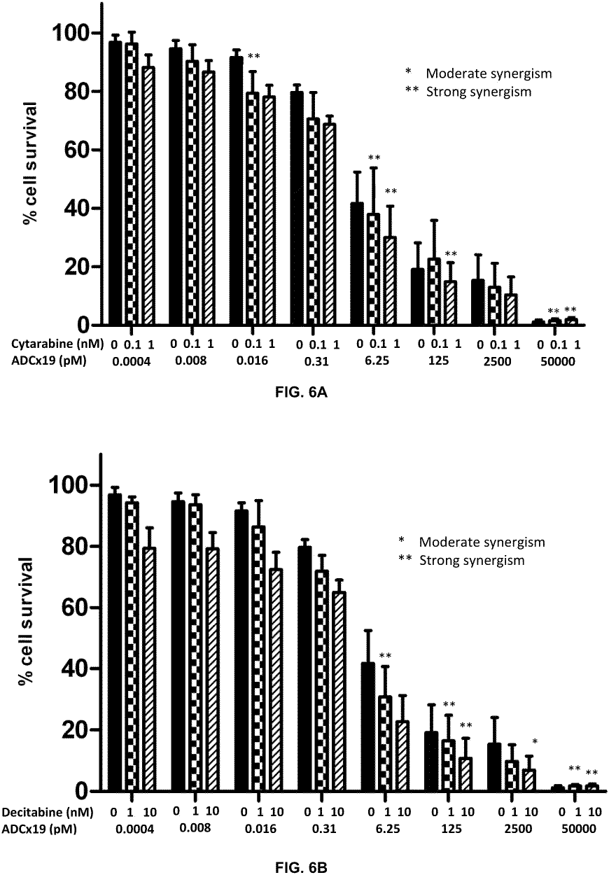

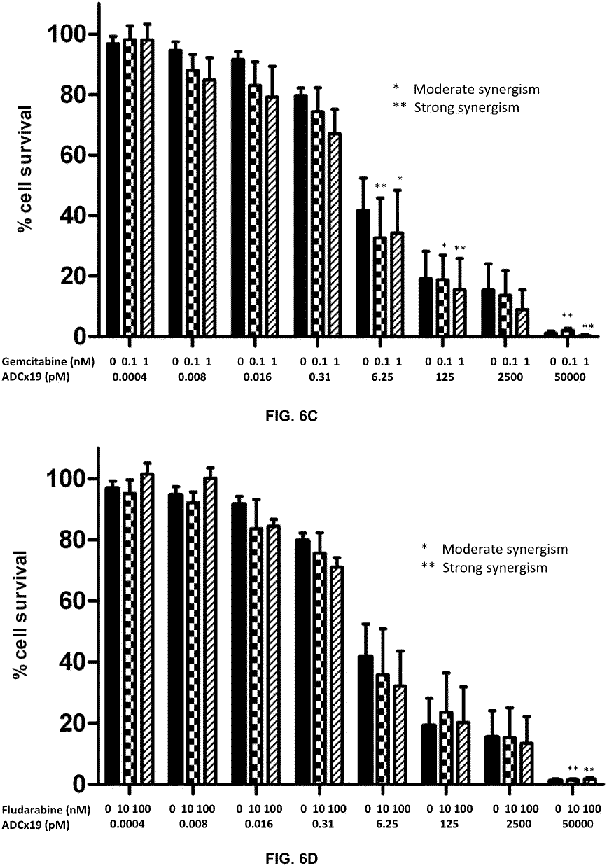

The results are shown in FIGS. 6A (Cytarabine), 6B (Decitabine), 6C (Gemcitabine), and 6D (Fludarabine), where * indicates moderate synergism and ** strong synergism as determined by CalcuSyn.

### Example 9: In Vitro Synergy in CD22+ve Ramos Cell Line of ADCx22 With Each of Cytarabine, Fludarabine, Decitabine, and Gemcitabine

Cells were plated on day 1 at 10,000 cells/well in 96-well plates, three replicates per experiment and total n of 3. Combination drug was added on day 2 and incubated for 24 hours at 37° C., 5% CO₂.

On day 3 ADCx22 was added to cells containing drug or media only as a control in the dosage range 0.005 pM - 50 nM at a 10 fold dilution and incubated for a further 4 days (3 x cell doubling time).

20 µl MTS was added to each well, and incubated for 2-3 hours under normal cell culture conditions. The OD was measured at 492 nm using a Thermo Labsystems Multiscan Ascent plate reader, and % growth calculated compared to the untreated control cells.

Growth curves were plotted using GraphPad Prism using the sigmoidal, 4PL, X is log (concentration) equation. IC50 values (dose of drug that inhibits growth by 50%) were determined. Percentage cell survival was converted into fraction affected (Fa) and the combination index (CI) for each dose calculated using CalcuSyn v2.11.

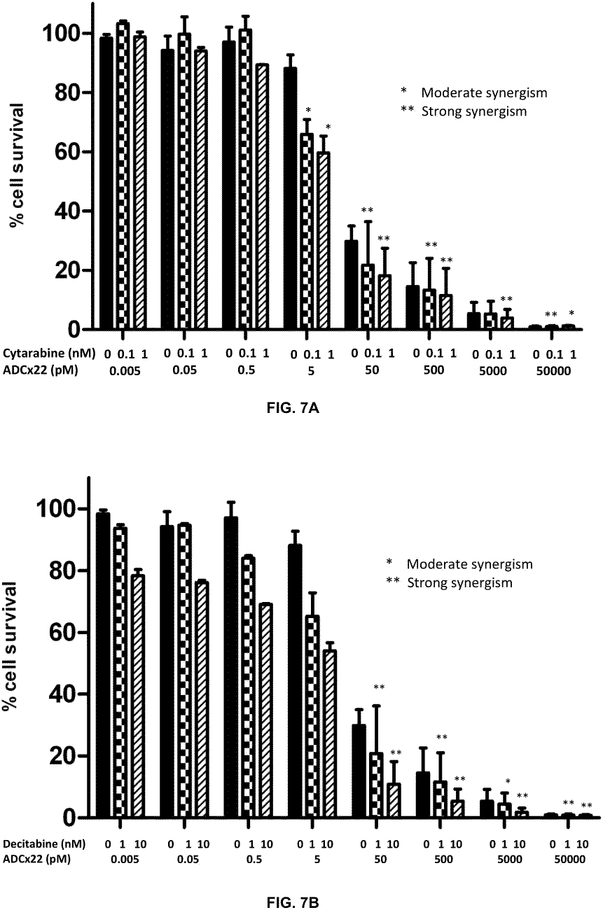

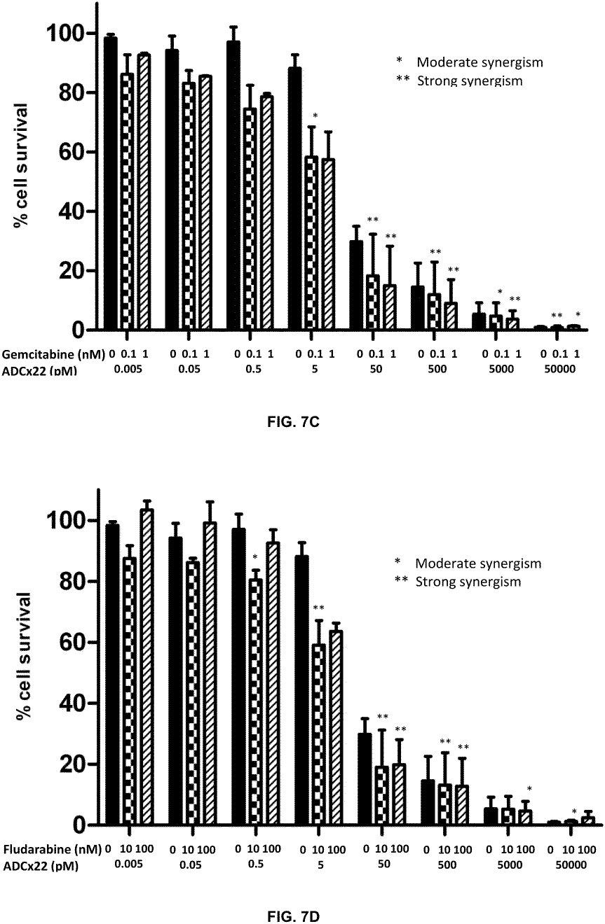

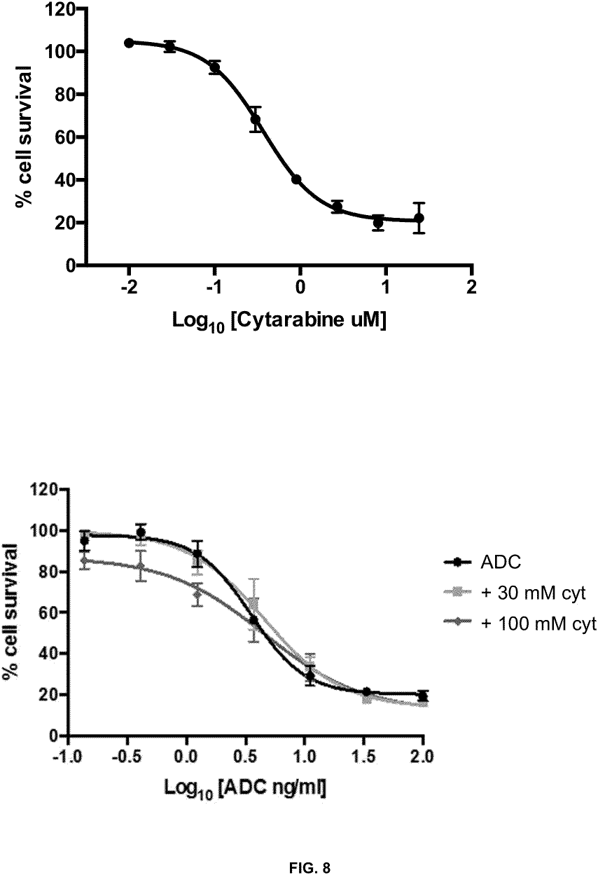

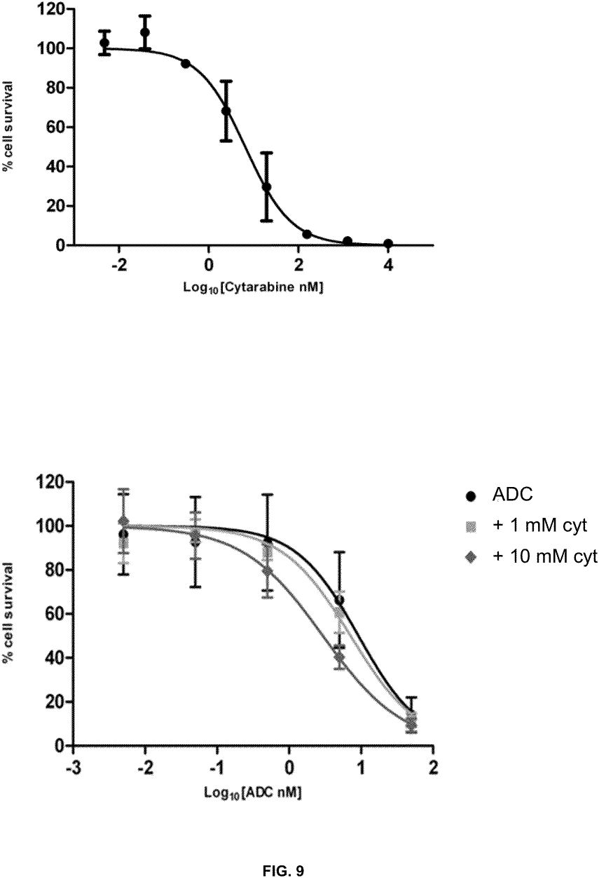

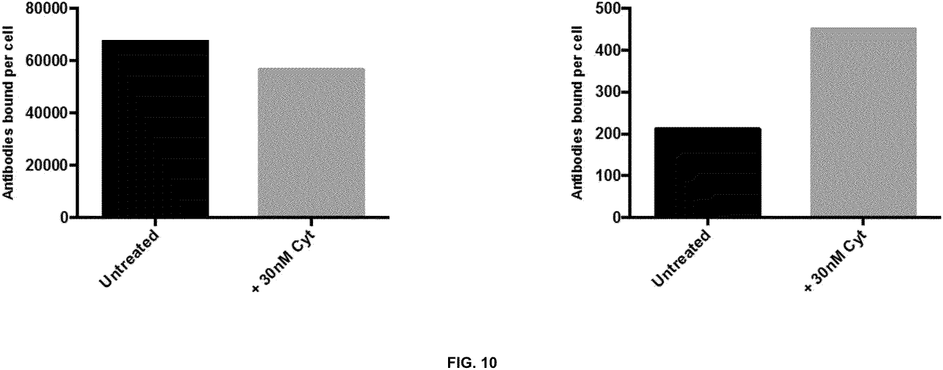

The results are shown in FIGS. 7A (Cytarabine), 7B (Decitabine), 7C (Gemcitabine), and 7D (Fludarabine), where * indicates moderate synergism and ** strong synergism as determined by CalcuSyn.

### EXAMPLE 10: Synergy Against CD19+ve Neoplastic Cells Between ADCx19 and Each of the Immunooncology (I/O) Secondary Agents PD1 Antagonists, PDL1 antAgonists, CTLA4 Antagonists, OX40 Agonists, and GITR Agonists

### PD1 Antagonists

To test whether a PBD-based ADC against CD19 combined with a PD1 antagonist shows additive or synergistic effect, the combination is tested in vivo in a syngeneic tumor model in immunocompetent mice (for CD19, potentially suitable models include A20, E.G7-OVA, EL4, C1498, L1210, P388). For this purpose, an antibody cross reactive with mouse CD19 is conjugated to a PBD warhead and this ADC is administered with the PD1 antagonist to mice grafted with a mouse tumor cell line expressing CD19. The ADC is administered before the PD1 antagonist, concomitantly with the PD1 antagonist, or after the PD1 antagonist, as decided by the experimenter.

Typically, the ADC is dosed as a single dose between 0.1 and 1 mg/kg, while the PD1 antagonist is dosed Q3d x 3 at doses between 1 and 10 mg/kg. Control groups include the ADC or PD1 antagonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or PD1 antagonist alone.

### PDL1 Antagonists

To test whether a PBD-based ADC against CD19 combined with a PDL1 antagonist shows additive or synergistic effect, the combination is tested in vivo in a syngeneic tumor model in immunocompetent mice. For this purpose, an antibody cross reactive with mouse CD19 is conjugated to a PBD warhead and this ADC is administered with the PDL1 antagonist to mice grafted with a mouse tumor cell line expressing CD19. The ADC is administered before the PDL1 antagonist, concomitantly with the PDL1 antagonist, or after the PDL1 antagonist, as decided by the experimenter.

Typically, the ADC is dosed as a single dose between 0.1 and 1 mg/kg, while the PD1 antagonist is dosed Q3d x 3 at doses between 1 and 10 mg/kg. Control groups include the ADC or PDL1 antagonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or PDL1 antagonist alone.

### CTLA4 Antagonists

To test whether a PBD-based ADC against CD19 combined with a CTLA4 antagonist shows additive or synergistic effect, the combination is tested in vivo in a syngeneic tumor model in immunocompetent mice. For this purpose, an antibody cross reactive with mouse CD19 is conjugated to a PBD warhead and this ADC is administered with the CTLA4 antagonist to mice grafted with a mouse tumor cell line expressing CD19. The ADC is administered before the CTLA4 antagonist, concomitantly with the CTLA4 antagonist, or after the CTLA4 antagonist, as decided by the experimenter.

Typically, the ADC is dosed as a single dose between 0.1 and 1 mg/kg, while the CLTA4 antagonist is dosed Q3d x 3 at doses between 1 and 10 mg/kg. Control groups include the ADC or CTLA4 antagonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or CTLA4 antagonist alone.

### OX40 Agonists

To test whether a PBD-based ADC against CD19 combined with a OX40 agonist shows additive or synergistic effect, the combination is tested in vivo in a syngeneic tumor model in immunocompetent mice. or this purpose, an antibody cross reactive with mouse CD19 is conjugated to a PBD warhead and this ADC is administered with the OX40 agonist to mice grafted with a mouse tumor cell line expressing CD19. The ADC is administered before the OX40 agonist, concomitantly with the OX40 agonist, or after the OX40 agonist, as decided by the experimenter.

Typically, the ADC is dosed as a single dose between 0.1 and 1 mg/kg, while the OX40 agonist is dosed Q3d x 3 at doses between 1 and 10 mg/kg. Control groups include the ADC or OX40 agonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or OX40 agonist alone.

### GITR Agonists

To test whether a PBD-based ADC against CD19 combined with a GITR agonist shows additive or synergistic effect, the combination is tested in vivo in a syngeneic tumor model in immunocompetent mice. For this purpose, an antibody cross reactive with mouse CD19 is conjugated to a PBD warhead and this ADC is administered with the GITR agonist to mice grafted with a mouse tumor cell line expressing CD19. The ADC is administered before the GITR agonist, concomitantly with the GITR agonist, or after the GITR agonist, as decided by the experimenter.

Typically, the ADC is dosed as a single dose between 0.1 and 1 mg/kg, while the GITR agonist is dosed Q3d x 3 at doses between 1 and 10 mg/kg. Control groups include the ADC or GITR agonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or GITR agonist alone.

### EXAMPLE 11: Synergy Against CD22+ve Neoplastic Cells Between ADCx22 and Each of the Immunooncology (I/O) Secondary Agents PD1 Antagonists, PDL1 Antagonists, CTLA4 Antagonists, OX40 Agonists, and GITR Agonists

### PD1 Antagonists

To test whether a PBD-based ADC against CD22 combined with a PD1 antagonist shows additive or synergistic effect, the combination is tested in vivo in a syngeneic tumor model in immunocompetent mice (for CD22, potentially suitable models include A20, E.G7-OVA, EL4, C1498, L1210, P388). For this purpose, an antibody cross reactive with mouse CD22 is conjugated to a PBD warhead and this ADC is administered with the PD1 antagonist to mice grafted with a mouse tumor cell line expressing CD22. The ADC is administered before the PD1 antagonist, concomitantly with the PD1 antagonist, or after the PD1 antagonist, as decided by the experimenter.

Typically, the ADC is dosed as a single dose between 0.1 and 1 mg/kg, while the PD1 antagonist is dosed Q3d x 3 at doses between 1 and 10 mg/kg. Control groups include the ADC or PD1 antagonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or PD1 antagonist alone.

### PDL1 Antagonists

To test whether a PBD-based ADC against CD22 combined with a PDL1 antagonist shows additive or synergistic effect, the combination is tested in vivo in a syngeneic tumor model in immunocompetent mice. For this purpose, an antibody cross reactive with mouse CD22 is conjugated to a PBD warhead and this ADC is administered with the PDL1 antagonist to mice grafted with a mouse tumor cell line expressing CD22. The ADC is administered before the PDL1 antagonist, concomitantly with the PDL1 antagonist, or after the PDL1 antagonist, as decided by the experimenter.

Typically, the ADC is dosed as a single dose between 0.1 and 1 mg/kg, while the PD1 antagonist is dosed Q3d x 3 at doses between 1 and 10 mg/kg. Control groups include the ADC or PDL1 antagonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or PDL1 antagonist alone.

### CTLA4 Antagonists

To test whether a PBD-based ADC against CD22 combined with a CTLA4 antagonist shows additive or synergistic effect, the combination is tested in vivo in a syngeneic tumor model in immunocompetent mice. For this purpose, an antibody cross reactive with mouse CD22 is conjugated to a PBD warhead and this ADC is administered with the CTLA4 antagonist to mice grafted with a mouse tumor cell line expressing CD22. The ADC is administered before the CTLA4 antagonist, concomitantly with the CTLA4 antagonist, or after the CTLA4 antagonist, as decided by the experimenter.

Typically, the ADC is dosed as a single dose between 0.1 and 1 mg/kg, while the CLTA4 antagonist is dosed Q3d x 3 at doses between 1 and 10 mg/kg. Control groups include the ADC or CTLA4 antagonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or CTLA4 antagonist alone.

### OX40 Agonists

To test whether a PBD-based ADC against CD22 combined with a OX40 agonist shows additive or synergistic effect, the combination is tested in vivo in a syngeneic tumor model in immunocompetent mice. or this purpose, an antibody cross reactive with mouse CD22 is conjugated to a PBD warhead and this ADC is administered with the OX40 agonist to mice grafted with a mouse tumor cell line expressing CD22. The ADC is administered before the OX40 agonist, concomitantly with the OX40 agonist, or after the OX40 agonist, as decided by the experimenter.

Typically, the ADC is dosed as a single dose between 0.1 and 1 mg/kg, while the OX40 agonist is dosed Q3d x 3 at doses between 1 and 10 mg/kg. Control groups include the ADC or OX40 agonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or OX40 agonist alone.

### GITR Agonists

To test whether a PBD-based ADC against CD22 combined with a GITR agonist shows additive or synergistic effect, the combination is tested in vivo in a syngeneic tumor model in immunocompetent mice. For this purpose, an antibody cross reactive with mouse CD22 is conjugated to a PBD warhead and this ADC is administered with the GITR agonist to mice grafted with a mouse tumor cell line expressing CD22. The ADC is administered before the GITR agonist, concomitantly with the GITR agonist, or after the GITR agonist, as decided by the experimenter.

Typically, the ADC is dosed as a single dose between 0.1 and 1 mg/kg, while the GITR agonist is dosed Q3d x 3 at doses between 1 and 10 mg/kg. Control groups include the ADC or GITR agonist alone. Tumor volumes and body weight is subsequently measured up to 60 days for all groups and the number of partially responding (PR), completely responding (CR) tumor free surviving (TFS mice is determined in each group.

Statistical analysis (typically a log-rank test) is performed to determine whether the mice treated with the combination have outperformed the mice treated with either ADC or GITR agonist alone.

1-49. (canceled)

50. A method for treating a disorder in an individual, the method comprising administering to the individual an effective amount of an anti-CD19-ADC and a secondary agent; wherein the secondary agent is a Bruton’s Tyrosine Kinase inhibitor (BTKi).

51. The method of claim 50, wherein the Bruton’s Tyrosine Kinase inhibitor (BTKi) is selected from Ibrutinib (Imbruvica), Acalabrutinib/ACP-196, ONO/GS-4059, Spebrutinib/AVL-292/CC-292, HM71224 (Poseltinib) and BGB-3111 (Zanubrutinib).

52. The method of claim 50, wherein the Bruton’s Tyrosine Kinase inhibitor (BTKi) is Ibrutinib (Imbruvica).

53. The method of claim 50, wherein the anti-CD19-ADC is ADCx19.

54. The method of claim 53, wherein the disorder is cancer.

55. The method of claim 53, wherein the disorder is selected from the group comprising: non-Hodgkin’s Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph-ALL).

56. The method of claim 55, wherein the disorder is DLBCL.

57. The method of claim 56, wherein the disorder is relapsed or refractory DLBCL.

58. The method of claim 57, wherein the individual has, or has been determined to have a cancer which expresses CD19 or CD19+ tumour-associated non-tumour cells, such as CD19+ infiltrating cells.

59. The method of claim 58, wherein the individual is undergoing or has undergone treatment with an anti-CD20 agent.

60. The method of claim 59, wherein the anti-CD20 agent is selected from obinutuzumab, Ibritumomab, rituximab, tiuxetan, tositumomab, Ofatumumab, Ocaratuzumab, Ocrelizumab, and Veltuzumab.

61. The method of claim 60, wherein the anti-CD20 agent is rituximab.

62. A kit comprising:
a first medicament comprising ADCx19;
a second medicament comprising a secondary agent, wherein the secondary agent is a Bruton’s Tyrosine Kinase inhibitor (BTKi); and
a package insert comprising instructions for administration of the first medicament according to the method of claim 1.

63. The kit of claim 62, wherein the Bruton’s Tyrosine Kinase inhibitor (BTKi) is Ibrutinib (Imbruvica).
